(19) United States
(12) Patent Application Publication
(10) Pub. No.: US 2025/0064955 A1
(43) Pub. Date: Feb. 27, 2025

(54) DRUG CONJUGATES AND METHODS OF PREPARING AND USING THE SAME

(71) Applicant: ADCENTRX THERAPEUTICS INC., San Diego, CA (US)

(72) Inventors: Richard Hui Li, San Diego, CA (US); Dong Jun Lee, San Diego, CA (US); Alexander Fann-Yan Chu-Kung, San Diego (UA); Erin Morgan Nye, San Diego, CA (US); Alexis Brooke Mahloch, San Diego, CA (US)

(21) Appl. No.: 18/704,398

(22) PCT Filed: Nov. 2, 2022
(86) PCT No.: PCT/US2022/048739

§ 371 Date: Apr. 24, 2024

Related U.S. Application Data
(60) Provisional application No. 63/275,403, filed on Nov. 3, 2021.

(51) Int. Cl. A61K 47/68; A61K 47/54; A61K 47/65; C07D 237/24; C07D 239/26; C07D 239/28; C07D 239/34; C07D 241/42

(52) U.S. Cl. CPC A61K 47/68031; A61K 47/545; A61K 47/65; A61K 47/6849; C07D 237/24; C07D 239/26; C07D 239/28; C07D 239/34; C07D 241/42

(57) ABSTRACT

The invention provides novel linkers, linker conjugates, and drug conjugates thereof, as well as methods of preparation and use thereof for treating various diseases and conditions.

### PRIORITY CLAIMS AND RELATED PATENT APPLICATIONS

This application claims the benefit of priority to U.S. Provisional Application Ser. No. 63/275,403, filed Nov. 3, 2021, the entire content of which is incorporated herein by reference.

### TECHNICAL FIELD OF THE INVENTION

The invention generally relates to novel compounds, methods of preparation, and therapeutic uses thereof. More particularly, the invention provides novel linkers, linker conjugates, and drug conjugates thereof, as well as methods of preparation and use thereof for treating various diseases and conditions.

### BACKGROUND OF THE INVENTION

Drug conjugates (e.g., antibody drug conjugates (ADCs)) can provide an effective means of delivering a drug to a targeted site in a tissue or organism. Twelve ADCs have been approved by the FDA to date, including gemtuzumab ozogamicin (Mylotarg™), the first ADC approved by the FDA in 2000. (See, e.g., Drago et al. 2021 Nature Reviews 18, 327-344; Mckertish et al. 2021 Biomedicines 9, 872; Khongorzui et al. 2020 Molecular Cancer Res. 18:3-19; Bross et al. 2001 Clin. Cancer Res. 7, 1490-1496; Hamann et al. 2002 Bioconjug. Chem. 13, 47-58; Lamb, 2017 Drugs 77, 1603-1610.) Lessons learned from the development of these ADCs highlight the importance of optimizing the drug to protein attachment method. Cysteine modification has gained popularity due to high nucleophilicity, selectivity towards electrophiles, and low natural abundance sulfhydryl group-bearing amino acid residues in naturally occurring proteins.

One conventional method employed in the design of ADCs includes the use of self-hydrolyzing maleimides for cysteine modification, as maleimides react rapidly and selectively with thiols (WIPO 2013/173337). While maleimide conjugation has led to stable drug-protein conjugation, the self-hydrolyzing maleimides generate acid species. These acid species can have unforeseen and deleterious effects on the properties of the resulting ADC. Stable conjugation between drug and protein has also been achieved through covalent conjugation through two cysteines (WIPO 2013/173391). While these methods have resulted in stable conjugation, the resulting drug to antibody ratio is low, leading to drug conjugates with a low drug load.

There remains a need for drug conjugates that feature a high degree of stability and drug loading without the generation of acidic species.

### SUMMARY OF THE INVENTION

Drug conjugates comprising a targeting moiety, a linker, and a drug moiety, methods of preparing the same, and methods of treating and/or preventing a condition using the same are provided herein.

In one aspect, the invention generally relates to a drug conjugate that comprise a targeting moiety, a linker moiety, and a drug moiety, wherein the drug moiety is conjugated to the linker which is conjugated to the targeting moiety, and wherein the linker moiety has the structural formula (I^{A}) or (II^{A}):

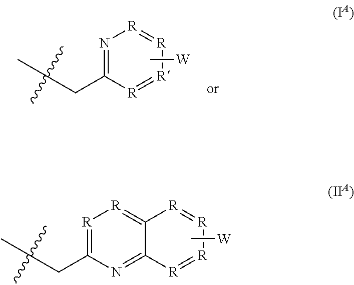

wherein:
each R is independently selected from N, CH, or C;
R′ is CH or C; and
W is selected from:

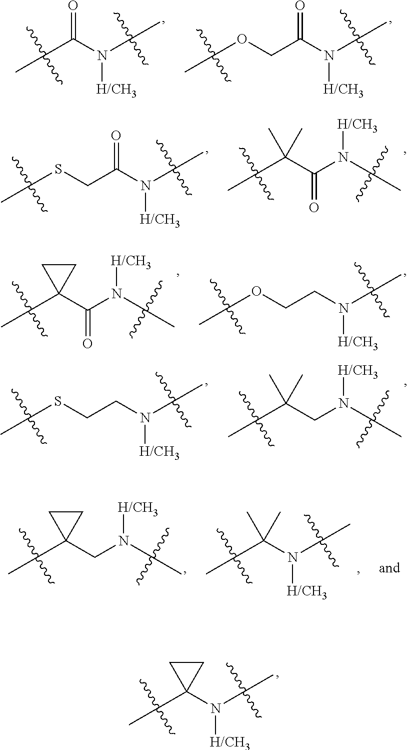

or a pharmaceutically acceptable salt thereof.

In yet another aspect, the invention generally relates to a pharmaceutical composition comprising a drug conjugate disclosed, or a pharmaceutically acceptable salt thereof, and a pharmaceutically acceptable excipient, carrier or diluent.

In yet another aspect, the invention generally relates to a compound that is useful for forming/preparing a linker-drug conjugate, a targeting-linker conjugate, or a targeting moiety-linker-drug conjugate, wherein the compound having a structure comprising formula (I) or (II):

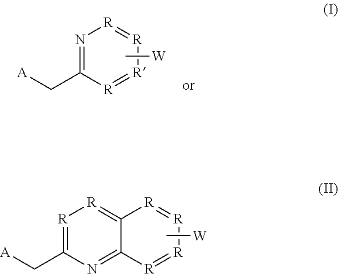

wherein:
A is Br or Cl;
each R is independently selected from N, CH, or C;
R′ is CH or C; and
W is selected from:

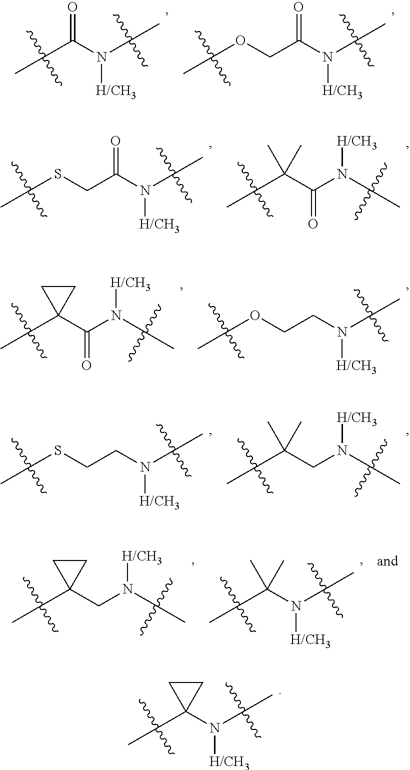

In yet another aspect, the invention generally relates to a compound useful for forming a conjugate of targeting moiety-linker-drug, wherein the compound having a structure comprising formula (I) or (II):

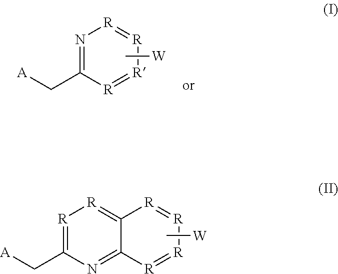

wherein:
A is Br or Cl;
each R is independently selected from N, CH, or C;
R′ is CH or C; and
W is selected from:

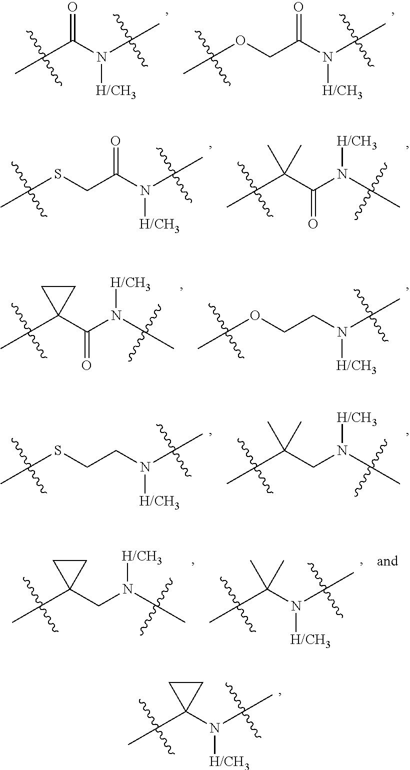

wherein W is covalently linked to a drug moiety, optionally via one or more spacer or linking moieties.

In yet another aspect, the invention generally relates to a compound that is useful for forming/preparing a conjugate of targeting moiety-linker-drug, wherein the compound has a structure comprising formula (I^{a}) or (II^{a}):

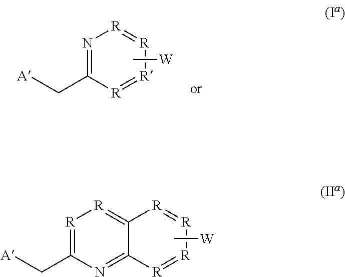

wherein:
A′ is conjugated to or comprises a targeting moiety;
each R is independently selected from N, CH, or C;
R′ is CH or C; and
W is selected from:

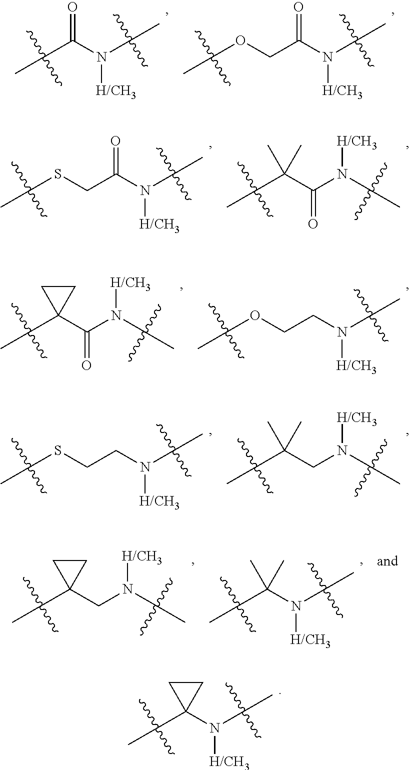

In yet another aspect, the invention generally relates to a composition comprising a compound of disclosed herein.

In yet another aspect, the invention generally relates to a method of preparing a drug conjugate comprising a targeting moiety, a linker, and a drug moiety. The method comprises: (a) providing a linker-drug moiety complex comprising a linker conjugated to a drug moiety; (b) providing a targeting moiety; and (c) conjugating the linker-drug moiety complex to the targeting moiety to form the drug conjugate, wherein the linker comprises a structure of:

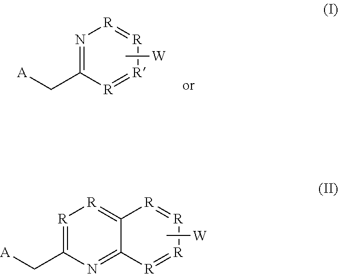

wherein:
A is Br or Cl;
each R is independently selected from N, CH, or C;
R′ is CH or C; and
W is selected from:

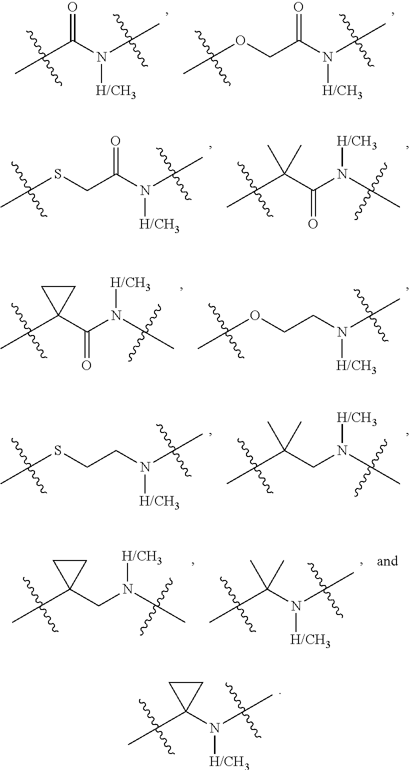

In yet another aspect, the invention generally relates to a method of preparing a linker-targeting moiety complex comprising a linker conjugated to a targeting moiety. The method comprising: (a) providing a linker moiety; (b) providing a targeting moiety; and (c) conjugating the linker to the targeting moiety to form the linker-targeting moiety complex, wherein the linker comprises a structure of formula (I) or (II):

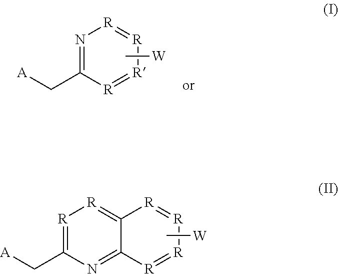

wherein:
A is Br or Cl;
each R is independently selected from N, CH, or C;
R′ is CH or C; and
W is selected from:

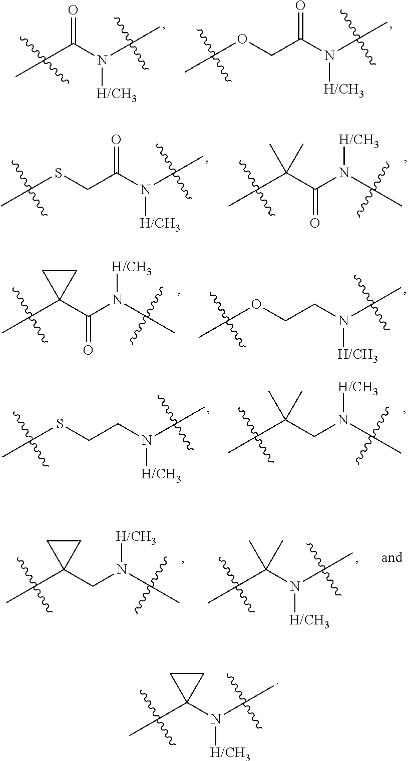

In yet another aspect, the invention generally relates to a method of treating and/or preventing a condition in a subject in need thereof, the method comprising administering to the subject a drug conjugate disclosed herein.

### BRIEF DESCRIPTION OF THE DRAWINGS

FIGS. 1A-1B show representative graphs related to viability studies of exemplary compounds in accordance with embodiments of the present disclosure.

FIG. 2 show exemplary conjugation methodology illustrated with compound 23 to Trastuzumab (DAR4).

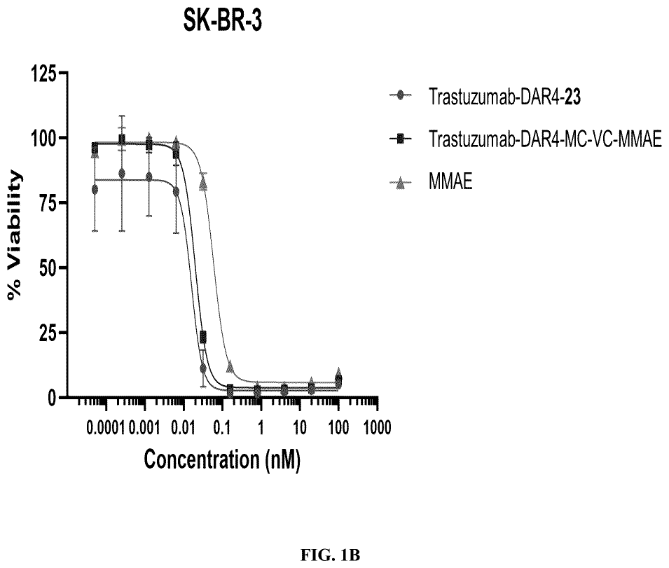

### DETAILED DESCRIPTION OF THE INVENTION

As set forth herein, novel linkers, linking methodologies and conjugates, and drug conjugates have been developed that possess unexpected advantages over prior art. Specifically, the conjugates disclosed herein exhibit a high degree of stability, and the conjugation process does not produce the deleterious acid species observed using conventional conjugation methods that employ self-hydrolyzing maleimides. Based on this disclosure, provided herein are novel linkers, linker conjugates, and drug conjugates comprising a drug moiety, a linker moiety, and a targeting moiety, components of these conjugates (e.g., linker or a portion thereof, linker-drug moiety, or linker-targeting moiety), methods of their preparation, kits comprising these drug conjugates and components thereof, and methods of using the drug conjugates and kits in the treatment of a disease or condition.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

The following terms, unless indicated otherwise according to the context wherein the terms are found, are intended to have the following meanings.

Ranges provided herein are understood to be shorthand for all of the values within the range. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, “at least” a specific value is understood to be that value and all values greater than that value.

In this specification and the appended claims, the singular forms “a,” “an,” and “the” include plural reference, unless the context clearly dictates otherwise.

Unless specifically stated or obvious from context, as used herein, the term “or” is understood to be inclusive.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Certain compounds of the present invention may exist in particular geometric or stereoisomeric forms. The present invention contemplates all such compounds, including cis- and trans-isomers, atropisomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the R- or S-configuration. For optically active compounds, it is often preferred to use one enantiomer to the substantial exclusion of the other enantiomer.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

A mixture of isomers can be separated on the basis of the physicochemical differences of the constituents, into the pure or substantially pure geometric or optical isomers, diastereomers, racemates, for example, by chromatography and/or fractional crystallization.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Where substituent groups are specified by their conventional chemical formulae, written from left to right, they equally encompass the chemically identical substituents that would result from writing the structure from right to left, e.g., —C(═O)—O— is equivalent to —O—C(═O)—.

Structures of compounds of the invention are limited by principles of chemical bonding known to those skilled in the art. Accordingly, where a group may be substituted by one or more of a number of substituents, such substitutions are selected so as to comply with principles of chemical bonding and to give compounds that are not inherently unstable and/or would be known to one of ordinary skill in the art as likely to be unstable under ambient conditions (e.g., aqueous, neutral, and several known physiological conditions).

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

Unless specifically stated or obvious from context, the term “about,” as used herein when referring to a measurable value such as an amount or concentration and the like, is meant to encompass variations of 20%, 10%, 5%, 1%, 0.5%, or even 0.1% of the specified amount.

The terms “treat,” “treating,” and “treatment” as used herein with regard to a condition refer to alleviating the condition partially or entirely; slowing the progression or development of the condition; eliminating, reducing, or slowing the development of one or more symptoms associated with the condition; or increasing progression-free or overall survival of the condition.

Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient's age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

The terms “prevent,” “preventing,” and “prevention” as used herein with regard to a condition refers to averting the onset of the condition or decreasing the likelihood of occurrence or recurrence of the condition, including in a subject that may be predisposed to the condition but has not yet been diagnosed as having the condition.

As used herein, the terms “disease”, “condition” or “disorder” are used interchangeably herein and refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

The term “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer. Thus, the terms “cancer” and “cancerous” refer to or describe the physiological condition in mammals that is typically characterized by unregulated cell growth. Examples of cancer include but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer.

The term “autoimmune disorder” may refer to a set of sustained organ-specific or systemic clinical symptoms and signs associated with altered immune homeostasis that is manifested by qualitative and/or quantitative defects of expressed autoimmune repertoires.

The term “infectious disease” may refer to any disease caused by an infectious organism such as a virus, bacteria, parasite, and/or fungus.

As used herein, the term “in need of” a treatment refers to a subject that would benefit biologically, medically or in quality of life from such a treatment.

The term “alkyl” describes an aliphatic hydrocarbon including straight chain and branched chain groups.

The term “heteroalkyl” describes an aliphatic hydrocarbon including straight chain and branched chain groups substituted with one or more atoms such nitrogen, oxygen, and sulfur.

The term “amino acid” as used herein refers to a molecule of the general formula NH₂—CHR—COOH, wherein “R” is one of a number of different side chains, or a residue within a peptide bearing the parent amino acid. Amino acids include naturally occurring amino acids with “R” being a substituent found in naturally occurring amino acids. “R” can also be a substituent that is not found in naturally occurring amino acids. The term “amino acid residue” refers to the portion of the amino acid which remains after losing a water molecule when it is joined to another amino acid. The term “modified amino acid” refers to an amino acid bearing an “R” substituent that does not correspond to one of the twenty genetically coded amino acids.

The term “antibody” as used herein refers to an immunoglobulin molecule or an immunologically active portion thereof that binds to a specific antigen, e.g., a cancer cell antigen, viral antigen, or microbial antigen. In those embodiments where the targeting moiety is an antibody and the antibody is a full-length immunoglobulin molecule, the antibody comprises two heavy chains and two light chains, with each heavy and light chain containing three complementary determining regions (CDRs). In those embodiments where the targeting moiety is an antibody and the antibody is an immunologically active portion of an immunoglobulin molecule, the antibody may be, for example, a Fab, Fab′, Fv, F(ab′)2, disulfide-linked Fv, scFv, single domain antibody (dAb), diabody, triabody, tetrabody, or linear antibody. Antibodies used as targeting moieties may be, for example, natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchlorate acid or with organic acids such as acetic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, p-toluenesulfonate, undecanoate, valerate salts, and the like. In some embodiments, organic acids from which salts can be derived include, for example, acetic acid, propionic acid, glycolic acid, pyruvic acid, lactic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “protein” and “polypeptide” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a polypeptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

Ranges recited herein are intended as continuous ranges, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein, and in all instances such ratios, ranges, and ranges of ratios represent various embodiments of the present disclosure.

### Targeting Moiety-Linker-Drug Conjugates

Provided herein in certain embodiments are drug conjugates comprising a linker, a drug moiety, and a targeting moiety. Also provided herein are components of these drug conjugates, including for example linkers, linker-drug moiety complexes, and linker-targeting moiety complexes.

In one aspect, the invention generally relates to a drug conjugate that comprise a targeting moiety, a linker moiety, and a drug moiety, wherein the drug moiety is conjugated to the linker which is conjugated to the targeting moiety, and wherein the linker moiety has the structural formula (I^{A}) or (II^{A}):

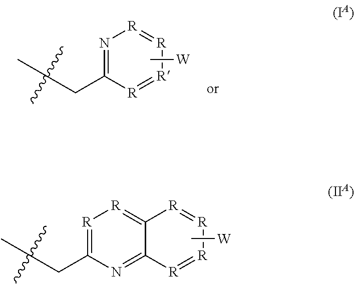

wherein:
each R is independently selected from N, CH, or C;
R′ is CH or C; and
W is selected from:

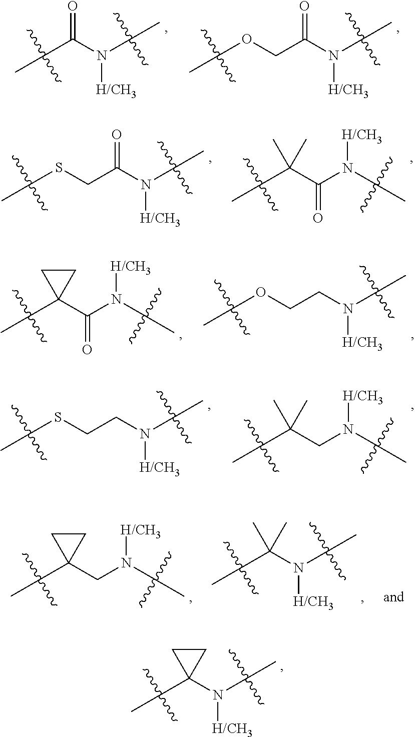

or a pharmaceutically acceptable salt thereof.

In certain embodiments, the linker has the structural formula (I^{B}) or (II^{B}):

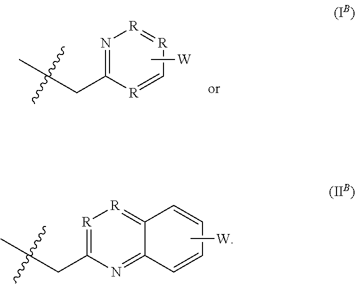

In certain embodiments, the linker has the structural formula (I^{C}):

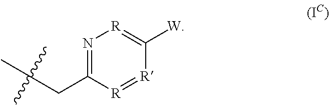

In certain embodiments, the linker comprises a spacer moiety and has the structural formula (III^{A}) or (IV^{A})

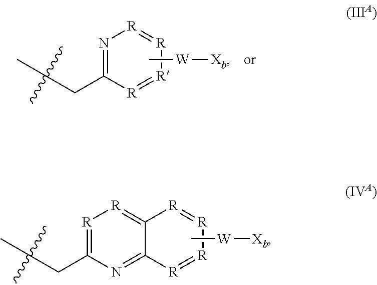

wherein X_{b} is the spacer moiety.

In certain embodiments, the spacer moiety is selected from the group consisting of an alkyl, a heteroalkyl, polyethylene glycol (PEG), and a peptide.

In certain embodiments, the linker comprises a spacer moiety and a polypeptide moiety and has the structural formula (V^{A}) or (VI^{A}):

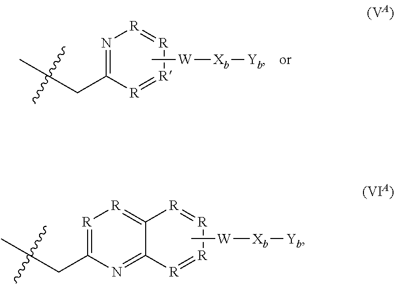

wherein Y_{b} is the polypeptide moiety.

In certain embodiments, the polypeptide moiety comprises 1, 2, 3, 4, 5 or 6 amino acids.

The amino acids may be natural and/or unnatural amino acids.

In certain embodiments, the linker comprises a spacer moiety, a polypeptide moiety, and a self-immolative moiety and has the structural formula (VII^{A}) or (VIII^{A}):

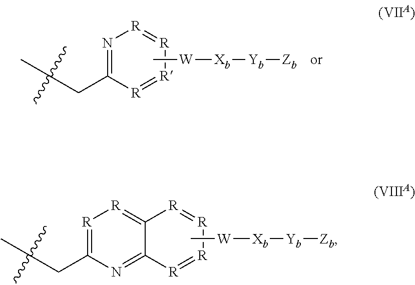

wherein Z_{b} is the self-immolative moiety.

In certain embodiments, the self-immolative moiety is selected from the group consisting of:

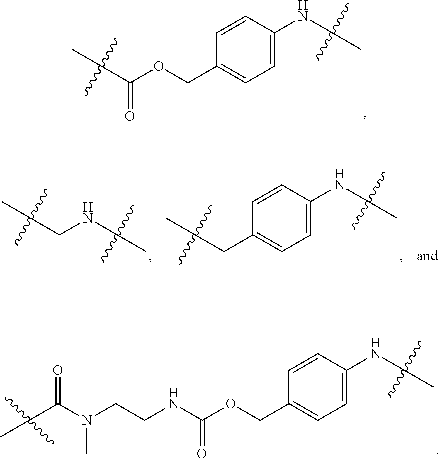

In certain embodiments, the linker comprises a group selected from:

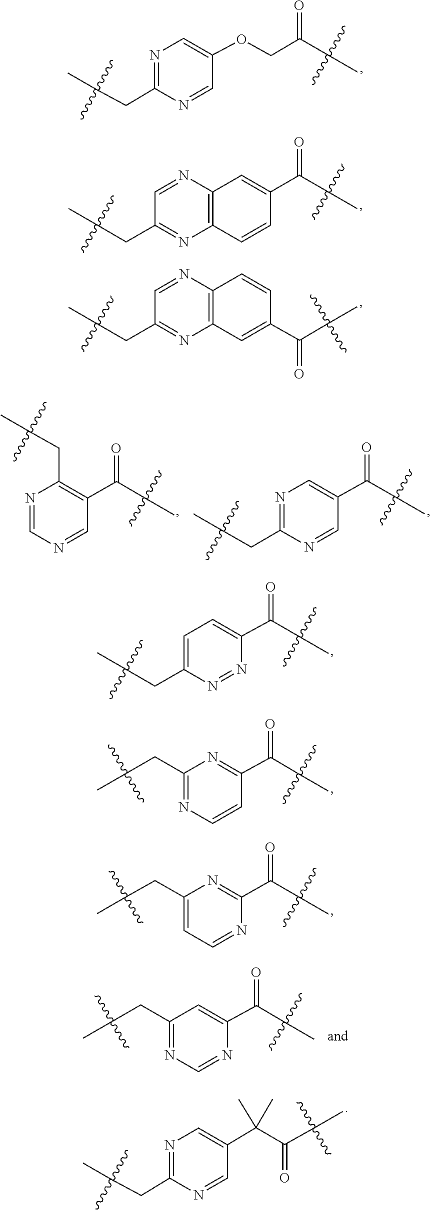

In certain embodiments, W is:

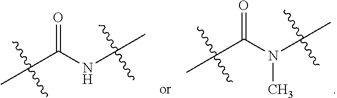

In certain embodiments, the drug moiety is a chemical agent selected from the group consisting of an antibiotic, an anti-cancer agent, a steroid, a TLR7/TLR9 antagonist, a polypeptide, a protein, and a nucleic acid.

In certain embodiments, the targeting moiety is selected from the group consisting of an antibody, small molecule, a peptide, a polypeptide, and a nucleic acid.

The drug conjugate may have a targeting moiety to drug moiety ratio of any suitable value, for example, from about 1:1 to about 1:16 (e.g., from about 1:1 to about 1:5, from about 1:5 to about 1:10, from about 1:10 to about 1:16).

In some embodiments, the drug conjugate has a structure of formula (XI):

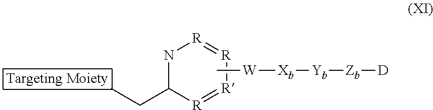

wherein:
each R is independently selected from N, CH, or C;
R′ is CH or C; and
W is selected from:

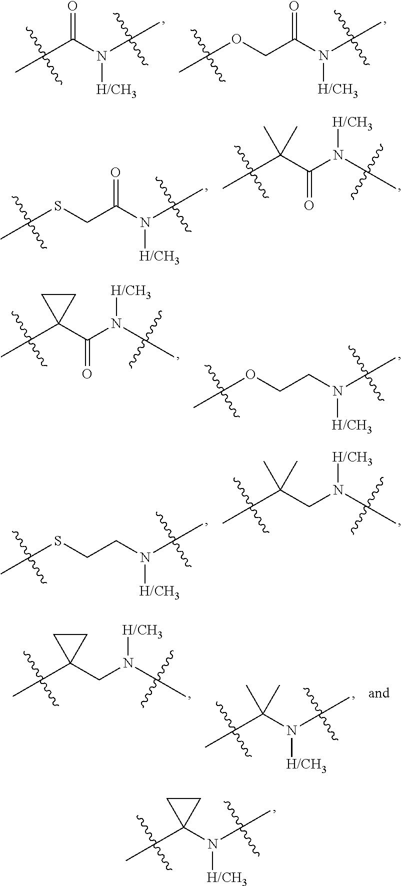

X_{b} is a spacer moiety,
Y_{b} is a polypeptide moiety,
Z_{b} is a self-immolative moiety, and
D is a drug moiety.

In some embodiments, the drug conjugate has a structure of formula (XII):

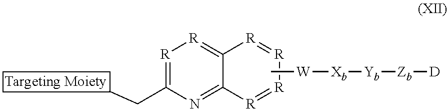

wherein:
each R is independently selected from N, CH, or C;
W is selected from:

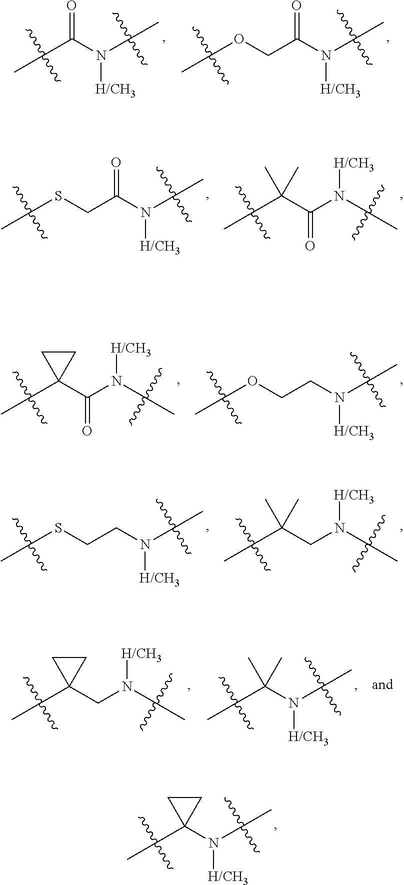

X_{b} is a spacer moiety,
Y_{b} is a polypeptide moiety,
Z_{b} is a self-immolative moiety, and
D is a drug moiety.

In certain embodiments, the drug conjugate comprises a linker including a polypeptide moiety and a self-immolative moiety. In some embodiments, the drug conjugate is one or more of:

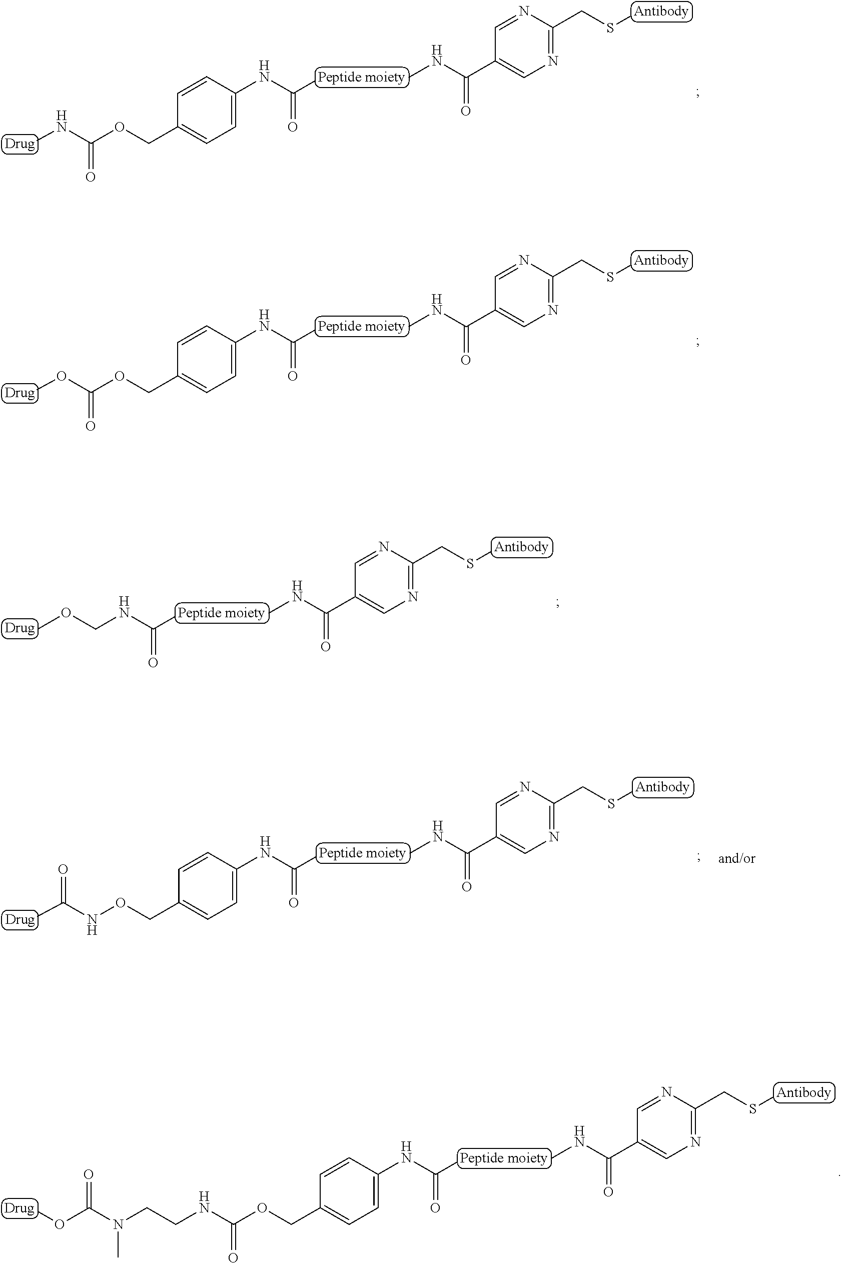

In another aspect, the invention generally relates to a composition comprising a drug conjugate disclosed herein.

In yet another aspect, the invention generally relates to a pharmaceutical composition comprising a drug conjugate disclosed, or a pharmaceutically acceptable salt thereof, and a pharmaceutically acceptable excipient, carrier or diluent.

### Linkers, Linker-Drug Conjugates, Targeting Moiety-Linker Conjugates

In yet another aspect, the invention generally relates to a compound that is useful for forming/preparing a linker-drug conjugate, a targeting-linker conjugate, or a targeting moiety-linker-drug conjugate, wherein the compound having a structure comprising formula (I) or (II):

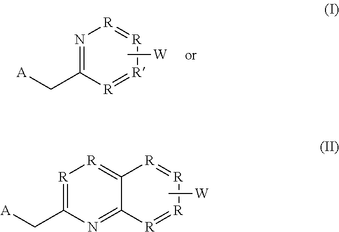

wherein:
A is Br or Cl;
each R is independently selected from N, CH, or C;
R′ is CH or C; and
W is selected from:

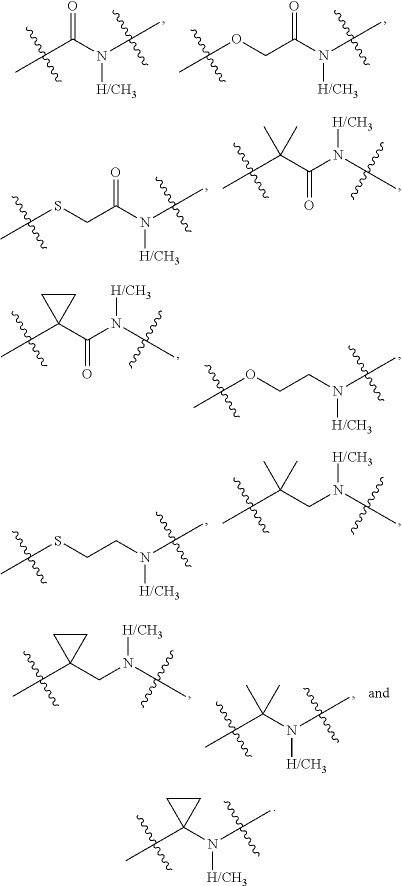

In certain embodiments, the compound has the structural formula (II^{B}) or (III^{B}):

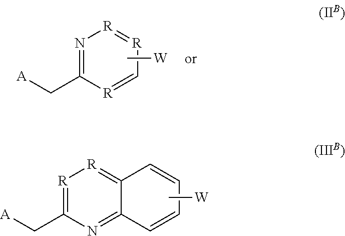

In certain embodiments, the compound further comprises a spacer moiety and has the structural formula:

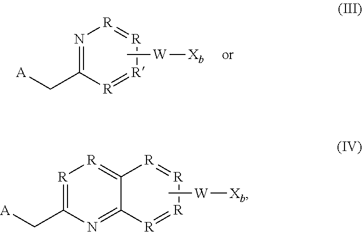

wherein X_{b} is the spacer moiety.

In certain embodiments, the spacer moiety is selected from the group consisting of an alkyl, a heteroalkyl, polyethylene glycol (PEG), and a peptide.

In certain embodiments, the compound comprises a spacer moiety and a polypeptide moiety, comprising the structural formula (V) or (VI):

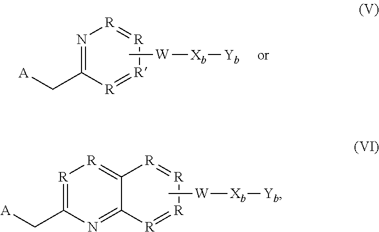

wherein Y_{b} is the polypeptide moiety.

In certain embodiments, the polypeptide moiety comprises 1, 2, 3, 4, 5 or 6 amino acids.

The amino acids may be natural and/or unnatural amino acids.

In certain embodiments, the compound comprises a spacer moiety, a polypeptide moiety, and a self-immolative moiety, having the structural formula:

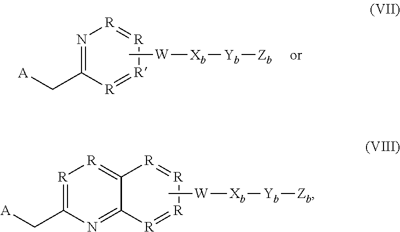

wherein Z_{b} is the self-immolative moiety.

In certain embodiments, the self-immolative moiety is selected from the group consisting of:

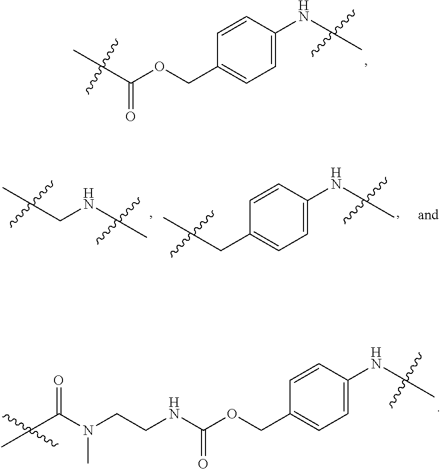

In certain embodiments, the compound has a structural formula selected from the group consisting of:

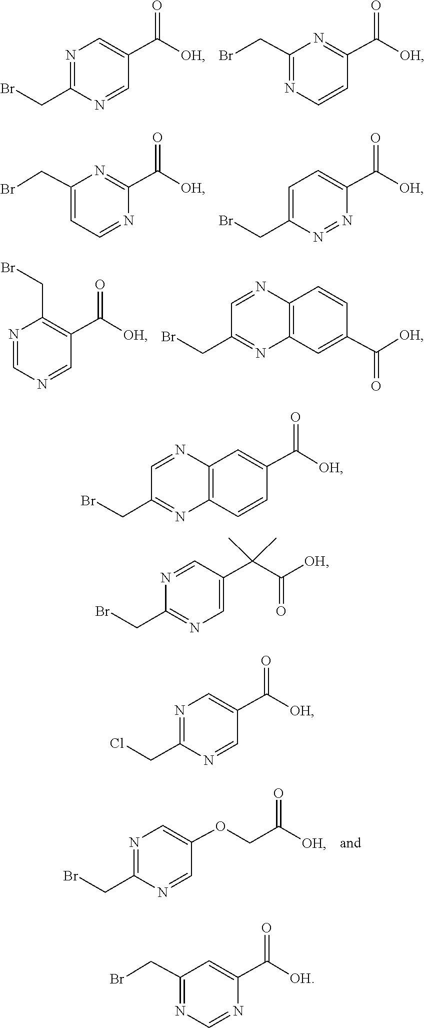

In certain embodiments, W is:

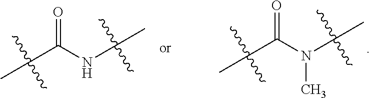

In yet another aspect, the invention generally relates to a compound useful for forming a conjugate of targeting moiety-linker-drug, wherein the compound having a structure comprising formula (I) or (II):

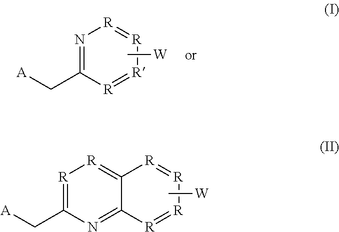

wherein:
A is Br or Cl;
each R is independently selected from N, CH, or C;
R′ is CH or C; and
W is selected from:

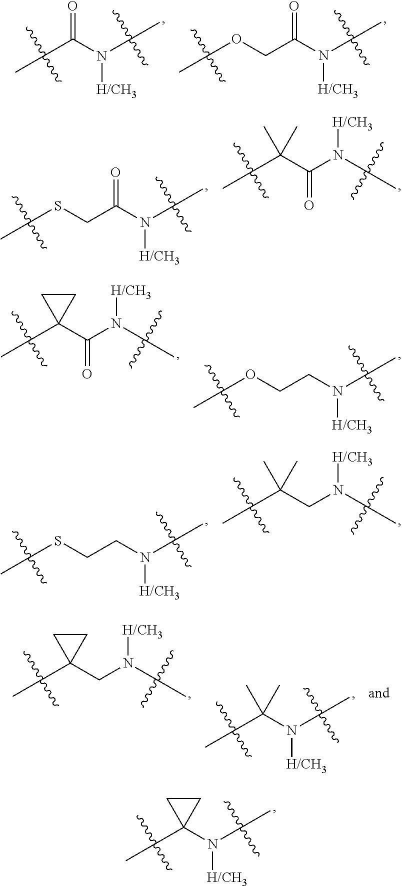

wherein W is covalently linked to a drug moiety, optionally via one or more spacer or linking moieties.

In certain embodiments, the compound has the structural formula (I^{D}) or (II^{D}):

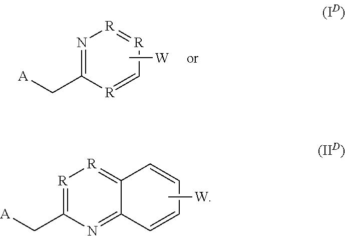

In certain embodiments, the compound comprises a spacer moiety and comprises the structural formula (III) or (IV):

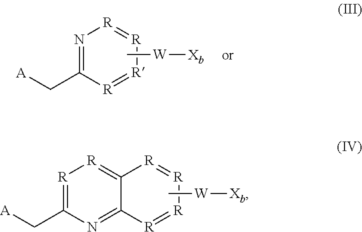

wherein X_{b} is the spacer moiety.

In certain embodiments, the spacer moiety is selected from the group consisting of an alkyl, a heteroalkyl, polyethylene glycol (PEG), and a peptide.

In certain embodiments, the compound comprises a spacer moiety and a polypeptide moiety and the structural formula (V) or (VI):

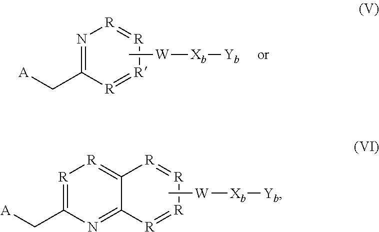

wherein Y_{b} is the polypeptide moiety.

In certain embodiments, the polypeptide moiety comprises 1, 2, 3, 4, 5 or 6 amino acids.

The amino acids may be natural and/or unnatural amino acids.

In certain embodiments, the compound comprises a spacer moiety, a polypeptide moiety, and a self-immolative moiety and comprises the structural formula (VII) or (VIII):

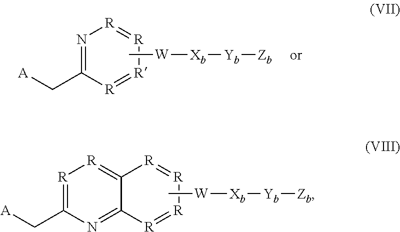

wherein Z_{b} is the self-immolative moiety.

In certain embodiments, the self-immolative moiety is selected from the group consisting of:

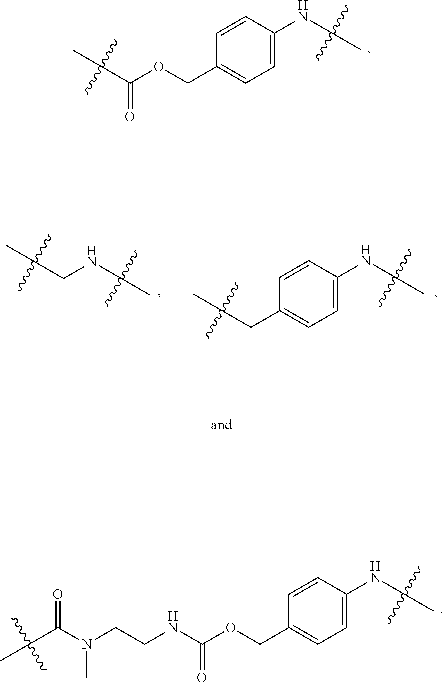

In certain embodiments, the compound has the structure selected from:

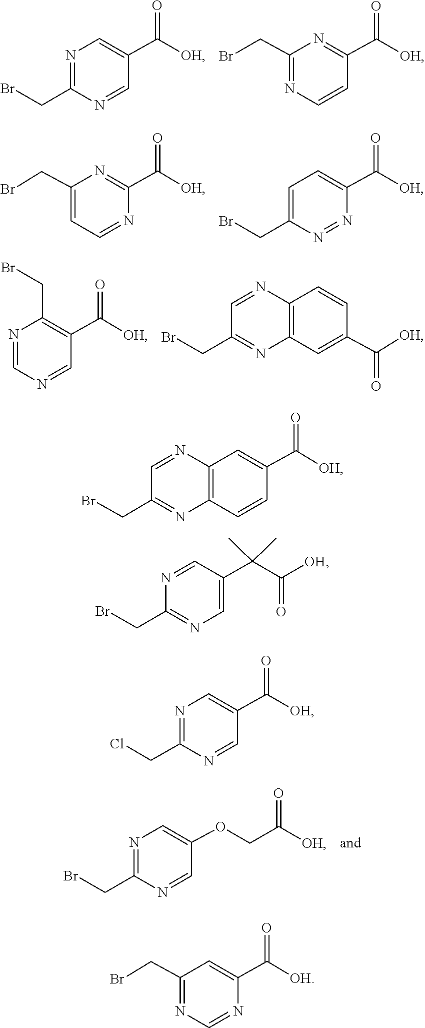

In certain embodiments, W is:

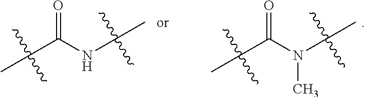

In certain embodiments, the drug moiety is a chemical agent selected from the group consisting of an antibiotic, an anti-cancer agent, a steroid, a TLR7/TLR9 antagonist, a polypeptide, a protein, and a nucleic acid.

In yet another aspect, the invention generally relates to a compound that is useful for forming/preparing a conjugate of targeting moiety-linker-drug, wherein the compound has a structure comprising formula (I^{a}) or (II^{a}):

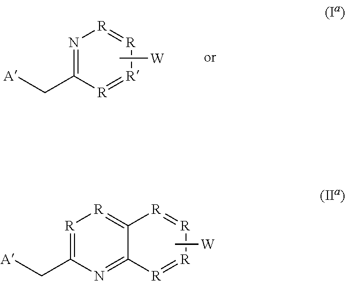

wherein:
A′ comprises or is conjugated to a targeting moiety;
each R is independently selected from N, CH, or C;
R′ is CH or C; and
W is selected from:

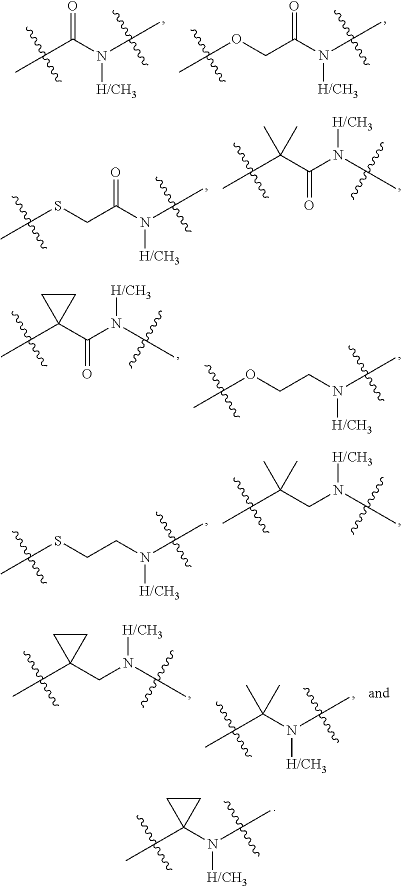

In certain embodiments, A′ comprises to a targeting moiety.

In certain embodiments, the compound has the structural formula (I^{b}) or (II^{b}):

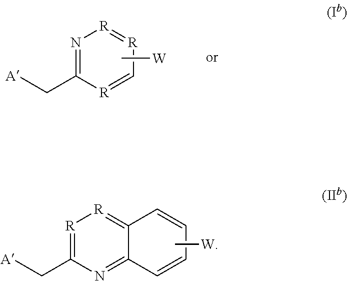

In certain embodiments, the compound comprises a spacer moiety and has a structure comprising formula (IIP^{a}) or (IV^{a}):

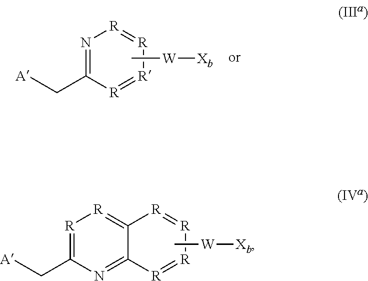

wherein X_{b} is the spacer moiety.

In certain embodiments, the spacer moiety is selected from the group consisting of an alkyl, a heteroalkyl, polyethylene glycol (PEG), and a peptide.

In certain embodiments, the compound comprises a spacer moiety and having a structure comprising formula (V^{a}) or (VI^{a}):

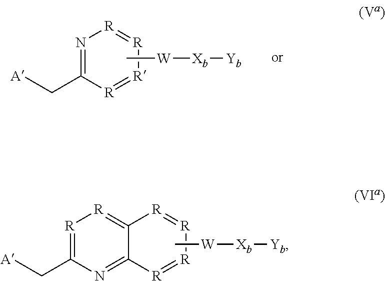

wherein Y_{b} is the polypeptide moiety.

In certain embodiments, the polypeptide moiety comprises 1, 2, 3, 4, 5 or 6 amino acids.

The amino acids may be natural and/or unnatural amino acids.

In certain embodiments, the compound comprises a spacer moiety, a polypeptide moiety, and a self-immolative moiety and has a structure comprising formula (VI^{a}), or (VII^{a}):

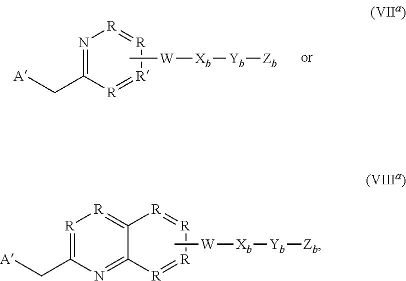

wherein Z_{b} is the self-immolative moiety.

In certain embodiments, the self-immolative moiety is selected from the group consisting of:

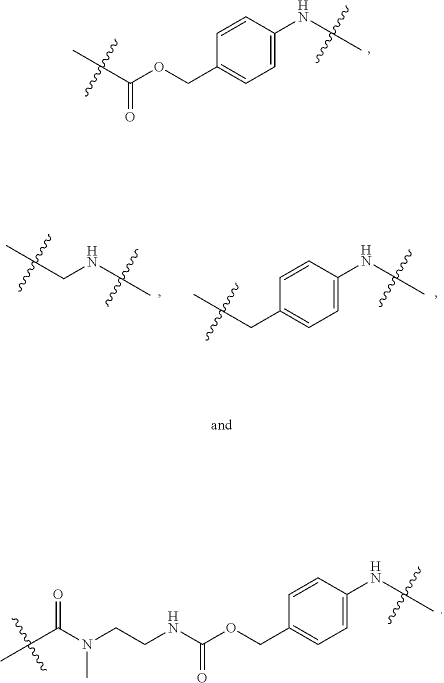

In certain embodiments, the compound comprising a linker moiety selected from:

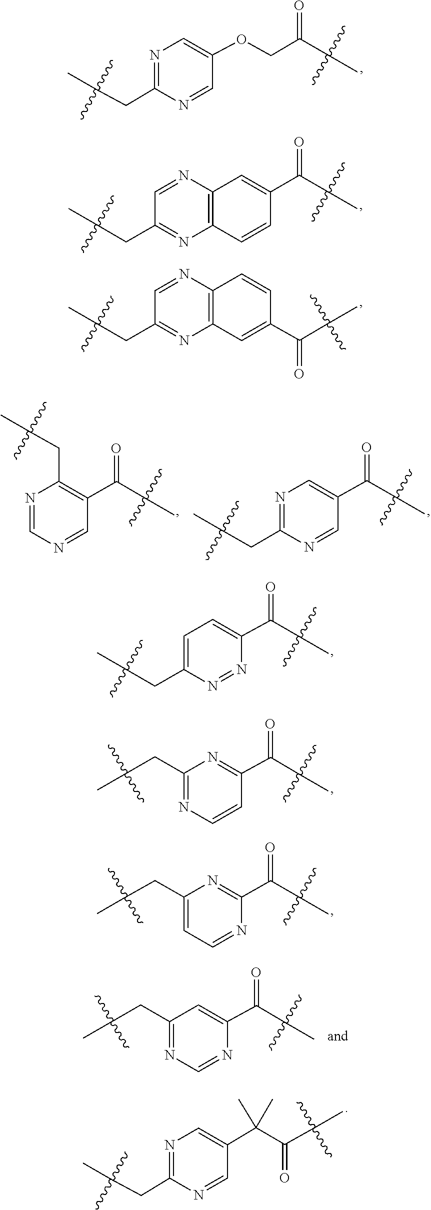

In certain embodiments, W is:

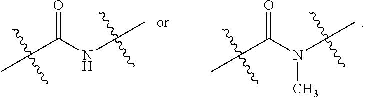

In certain embodiments, the targeting moiety is selected from the group consisting of an antibody, small molecule, a peptide, a polypeptide, and a nucleic acid.

In yet another aspect, the invention generally relates to a composition comprising a compound of disclosed herein.

### Methods of Preparation

In yet another aspect, the invention generally relates to a method of preparing a drug conjugate comprising a targeting moiety, a linker, and a drug moiety. The method comprises: (a) providing a linker-drug moiety complex comprising a linker conjugated to a drug moiety; (b) providing a targeting moiety; and (c) conjugating the linker-drug moiety complex to the targeting moiety to form the drug conjugate, wherein the linker comprises a structure of:

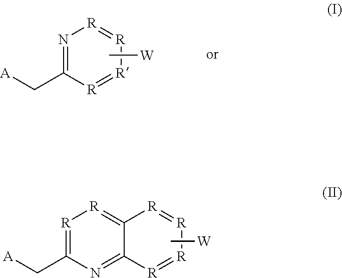

wherein:
A is Br or Cl;
each R is independently selected from N, CH, or C;
R′ is CH or C; and
W is selected from:

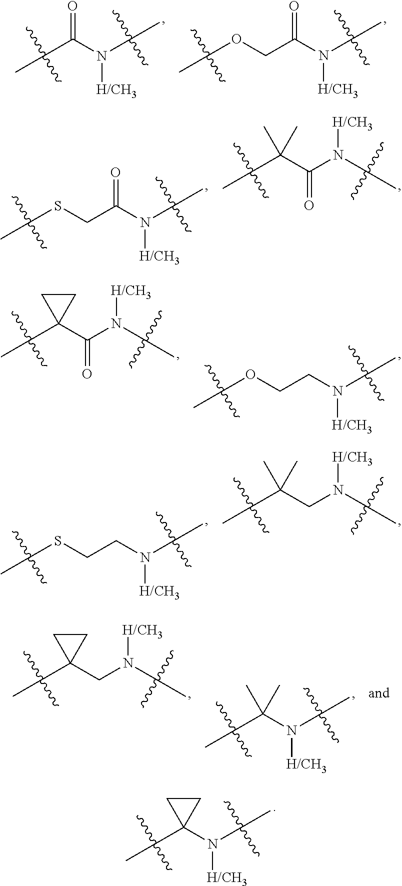

In certain embodiments, the linker has the structural formula (I^{D}) or (II^{D}):

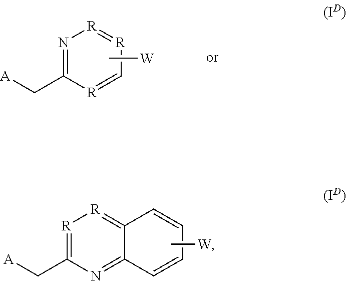

In certain embodiments, the linker comprises a spacer moiety and has a structure comprising a formula (III) or (IV):

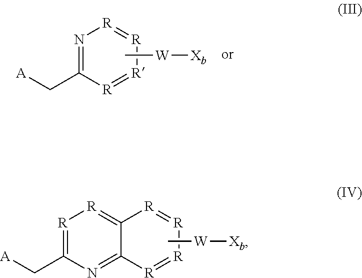

wherein X_{b} is the spacer moiety.

In certain embodiments, the spacer moiety is selected from the group consisting of an alkyl, a heteroalkyl, polyethylene glycol (PEG), and a peptide.

In certain embodiments, the linker comprises a spacer moiety and a polypeptide moiety and has a structure comprising formula (V) or (VI):

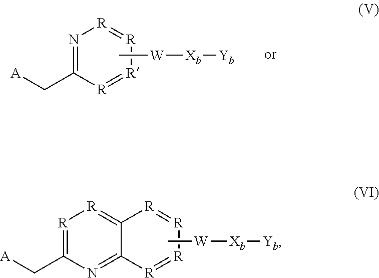

wherein Y_{b} is the polypeptide moiety.

In certain embodiments, the polypeptide moiety comprises 1, 2, 3, 4, 5 or 6 amino acids.

The amino acids may be natural and/or unnatural amino acids.

In certain embodiments, the linker includes a spacer moiety, a polypeptide moiety, and a self-immolative moiety and has a structure comprising a formula (VII) or (VIII):

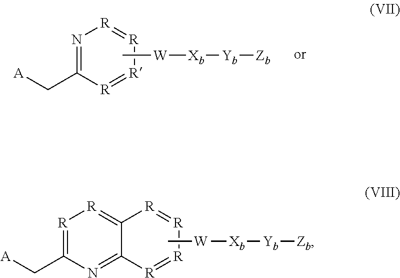

wherein Z_{b} is the self-immolative moiety.

In certain embodiments, the self-immolative moiety is selected from the group consisting of:

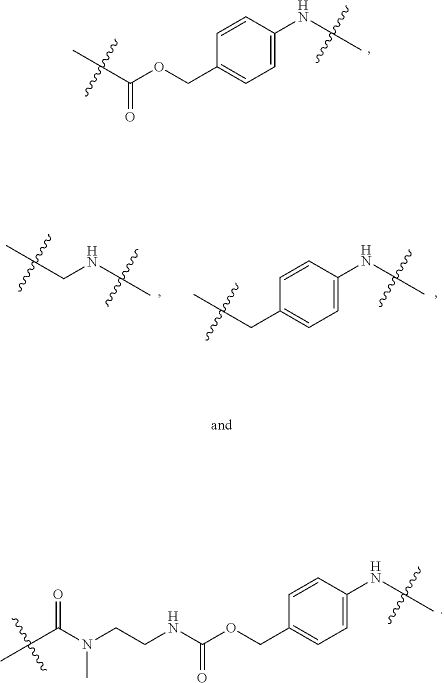

In certain embodiments, the linker is selected from the group consisting of:

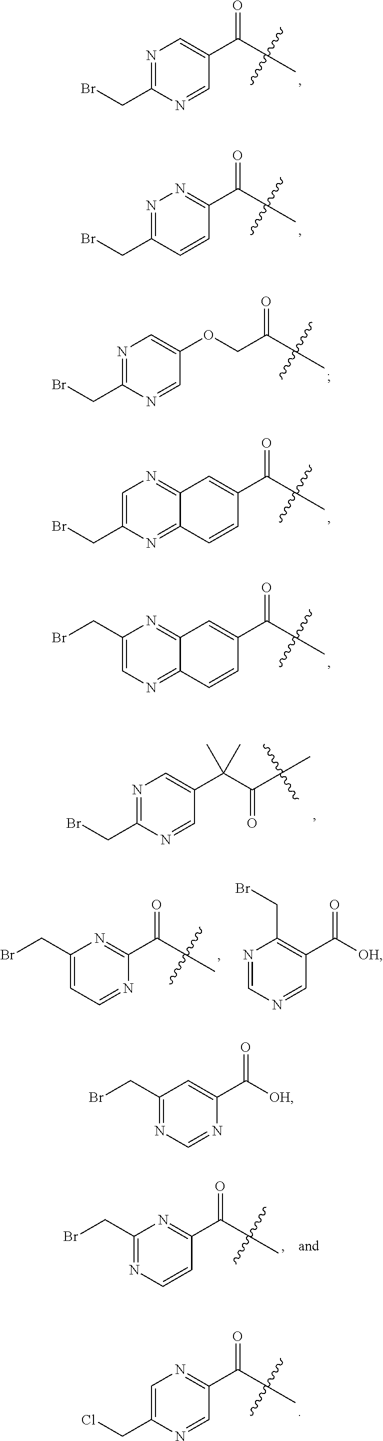

In certain embodiments, W is:

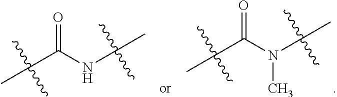

In certain embodiments, the targeting moiety comprises a cysteine residue.

In certain embodiments, the method further comprises reducing the cysteine reside to form a sulfhydryl and reacting the sulfhydryl with the linker-drug moiety complex to form the drug conjugate.

In yet another aspect, the invention generally relates to a method of preparing a linker-targeting moiety complex comprising a linker conjugated to a targeting moiety. The method comprising: (a) providing a linker moiety; (b) providing a targeting moiety; and (c) conjugating the linker to the targeting moiety to form the linker-targeting moiety complex, wherein the linker comprises a structure of formula (I) or (II):

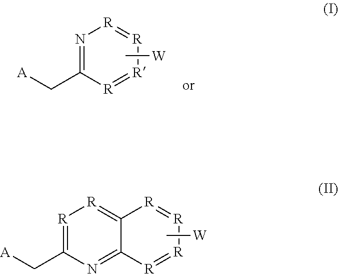

wherein:
A is Br or Cl;
each R is independently selected from N, CH, or C;
R′ is CH or C; and
W is selected from:

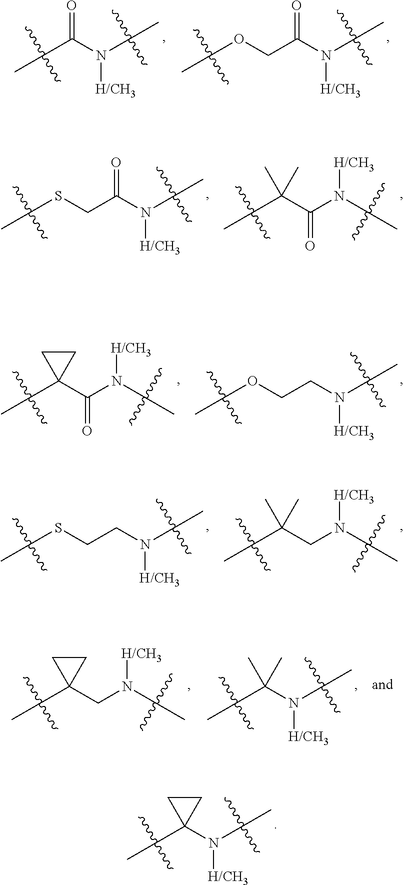

In certain embodiments, W is:

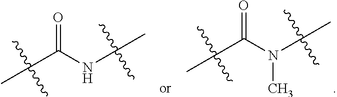

In some embodiments, the targeting moiety comprises a sulfhydryl moiety. In some embodiments, the methods comprise providing a targeting moiety comprising a cysteine residue and reducing the cysteine residue to form the sulfhydryl moiety. In some embodiments, the methods comprise conjugating the linker portion to the targeting moiety via the sulfhydryl group.

In some embodiments, the methods for preparing a drug conjugate proceed according to the exemplary reaction shown in Scheme 1:

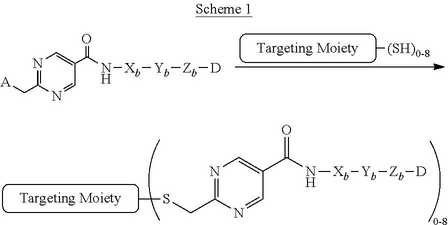

wherein:
A is Br or Cl;
X_{b} is a spacer moiety,
Y_{b} is a polypeptide moiety,
Z_{b} is a self-immolative moiety, and
D is a drug moiety.

In some embodiments, the targeting moiety is an antibody comprising a cysteine residue, and the methods comprise reducing the cysteine residue to form a sulfhydryl group and reacting the sulfhydryl group with one or more linker portions described herein.

In certain embodiments, the targeting moiety is an antibody fragment comprising a cysteine residue, and the methods comprise reducing the cysteine residue to form a sulfhydryl group and reacting the sulfhydryl group with one or more linker portions described herein.

In certain embodiments, the targeting moiety is a protein ligand comprising a cysteine, and the methods comprise reducing the cysteine residue to form a sulfhydryl group and reacting the sulfhydryl group with one or more linker portions described herein.

In certain embodiments, the targeting moiety is a protein scaffold comprising a cysteine, and the methods comprise reducing the cysteine residue to form a sulfhydryl group and reacting the sulfhydryl group with one or more linker portions described herein.

In certain embodiments, the targeting moiety is a small molecule comprising a cysteine, and the methods comprise reducing the cysteine residue to form a sulfhydryl group and reacting the sulfhydryl group with one or more linker portions described herein.

In certain embodiments, conjugating the linker portion to the targeting moiety produces no deleterious side products. Non-limiting examples of deleterious side-products include acids, bases, or combination thereof.

In certain embodiments, the drug conjugate has a high drug loading. For example, in some embodiments, a molar ratio of the targeting moiety to the drug moiety is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15 or about 1:16.

In certain embodiments, the drug conjugate is stable in vivo (e.g., does not undergo a deconjugation process).

### Use of Compounds and Drug Conjugates

In yet another aspect, the invention generally relates to a method of treating and/or preventing a condition in a subject in need thereof, the method comprising administering to the subject a drug conjugate disclosed herein.

In some embodiments, the condition is cancer, an autoimmune disorder, or an infectious disease.

In some embodiments, the methods of treating and/or preventing a condition in a subject in need thereof comprise administering to the subject one or more drug conjugates of the present disclosure, where upon administration to the subject the drug moiety is released from the drug conjugate. In certain embodiments, the drug moiety is released from the drug conjugate by self-immolative cleavage of the self-immolative moiety.

In some embodiments, the methods for treating and/or preventing a condition comprise administering to the subject one or more drug conjugates of the present disclosure, where upon administration to the subject the drug moiety is released from the drug conjugate according to exemplary Schemes 2-6, each of which feature a different self-immolative moiety.

In some embodiments, proteolytic cleavage of the drug conjugate proceeds according to Scheme 2:

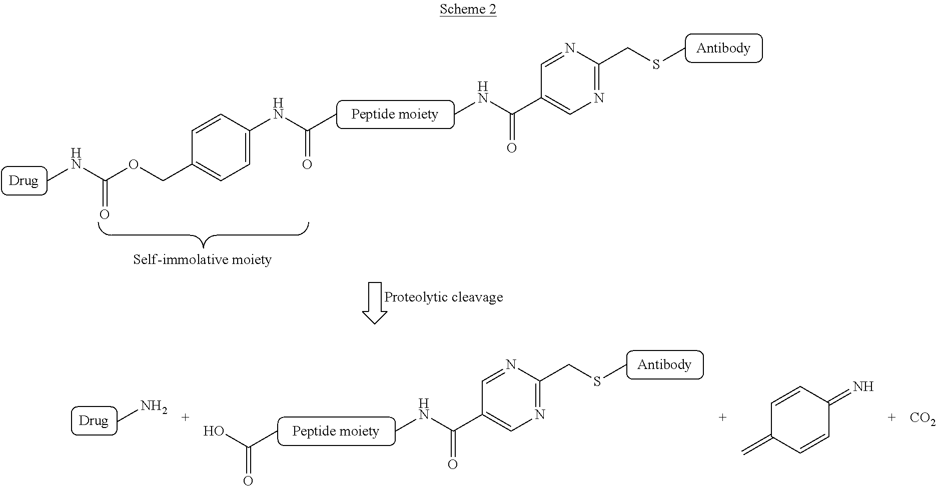

In some embodiments, the proteolytic cleavage of the drug conjugate proceeds according to Scheme 3:

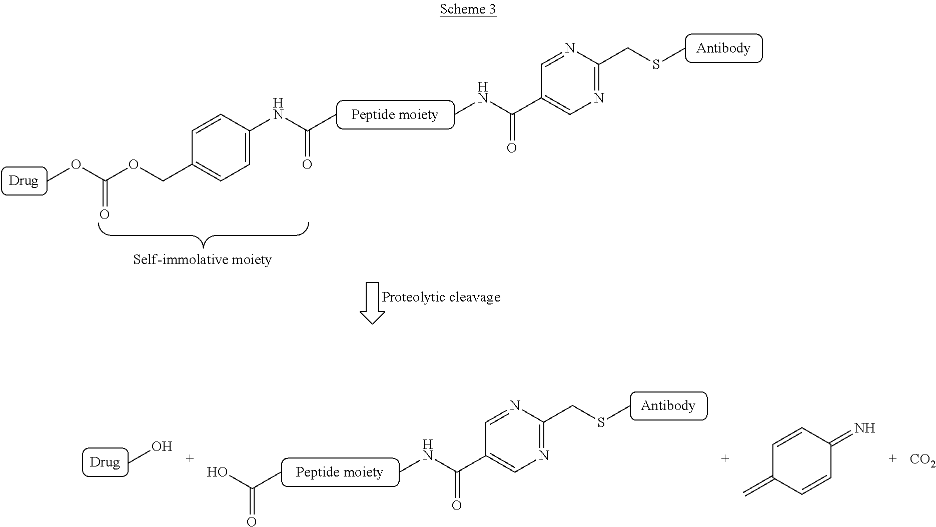

In some embodiments, the proteolytic cleavage of the drug conjugate proceeds according to Scheme 4:

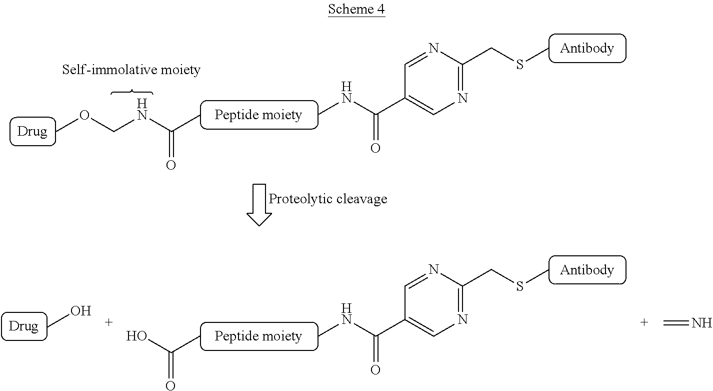

In some embodiments, the proteolytic cleavage of the drug conjugate proceeds according to Scheme 5:

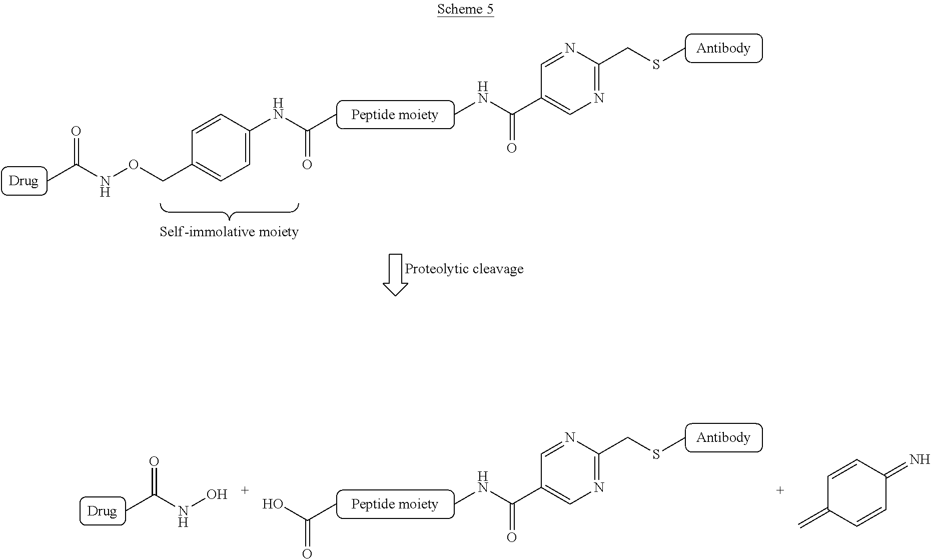

In some embodiments, the proteolytic cleavage of the drug conjugate proceeds according to Scheme 6:

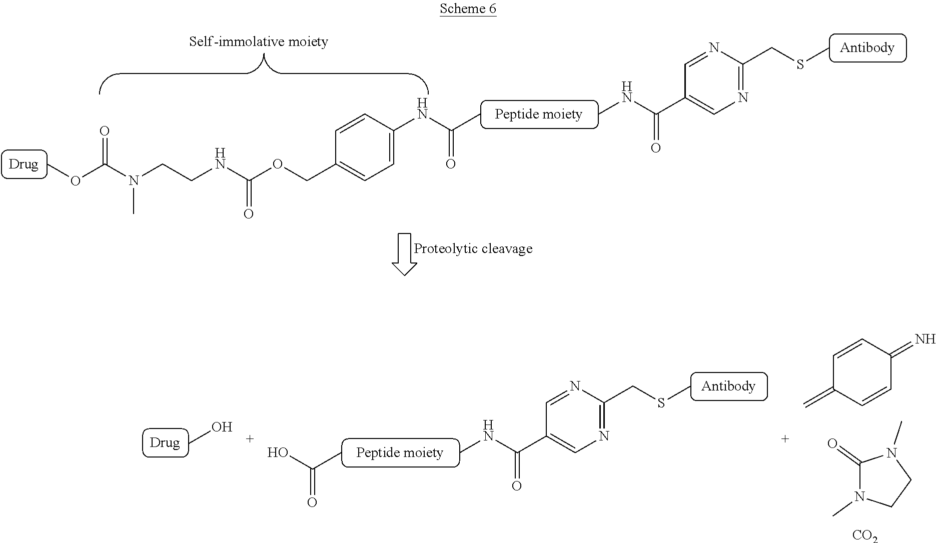

In some embodiments, the condition being treated and/or prevented is cancer. In some of these embodiments, the cancer is adrenal cancer, anal cancer, basal and squamous cell skin cancer, bile duct cancer, bladder cancer, bone cancer, brain and spinal cord tumors (e.g., astrocytoma, glioblastoma multiforme, meningioma), breast cancer, cervical cancer, colorectal cancer, endometrial cancer, esophagus cancer, Ewing family of tumors, eye cancer (ocular melanoma), gallbladder cancer, gastrointestinal neuroendocrine (carcinoid) tumors, gastrointestinal stromal tumor (gist), gestational trophoblastic disease, Kaposi sarcoma, kidney cancer, laryngeal and hypopharyngeal cancer, liver cancer, lung cancer, lung carcinoid tumor, malignant mesothelioma, melanoma skin cancer, Merkle cell skin cancer, nasal cavity and paranasal sinuses cancer, nasopharyngeal cancer, neuroblastoma, non-small cell lung cancer, neoplasm of the central nervous system (CNS), oral cavity and oropharyngeal cancer, osteosarcoma, ovarian cancer, pancreatic cancer, pancreatic neuroendocrine tumor (net), penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer, uterine sarcoma, vaginal cancer, vulvar cancer, Waldenstrom macroglobulinemia, Wilms tumor, squamous cell cancer, cancers of unknown primary (CUP), environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma.

In some of these embodiments, the cancer is a hematologic malignancy. In some embodiments, the hematologic malignancy is chronic lymphocytic leukemia (CLL), acute leukemia, acute lymphoid leukemia (ALL), B-cell acute lymphoid leukemia (B-ALL), T-cell acute lymphoid leukemia (T-ALL), T-cell lymphoma, B-cell lymphoma, chronic myelogenous leukemia (CML), acute myelogenous leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In other embodiments, the cancer is a human hematologic malignancy such as myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplastic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some embodiments, the cancer comprises a solid tumor. In some embodiments, the solid tumor is lung cancer, colorectal cancer, breast cancer, pancreatic cancer, gallbladder cancer, brain and spinal cord cancer, head and neck cancer, skin cancers, testicular cancer, prostate cancer, ovarian cancer, renal cell carcinoma (RCC), bladder cancer and hepatocellular carcinoma (HCC).

Methods according to this disclosure may further include administering one or more drug conjugates provided herein to treat and/or prevent cancer in a combination therapy. For example, in certain embodiments, a combination therapy comprises administering one or more drug conjugates (concurrently or sequentially) with a chemotherapeutic agent. In further embodiments, a combination therapy comprises administering one or more drug conjugates with a secondary therapy, such as chemotherapeutic agent, a radiation therapy, a surgery, an antibody, or any combination thereof. In some embodiments, administration one or more drug conjugates in combination with radiation therapy, antibody agent and/or chemotherapeutic agents results in an enhancement of said radiation therapy, antibody agent and/or chemotherapeutic agents such that, for example, a smaller dosage of the radiation, antibody therapy and/or chemotherapy may be effective for treatment and/or prevention.

In some embodiments, the condition being treated and/or prevented is an autoimmune disorder. In some of these embodiments, the autoimmune disorder is one or more of Th2 lymphocyte disorders, Th1 lymphocyte disorders, activated B lymphocyte disorders, active chronic hepatitis, Addison's disease, allergic alveolitis, allergic reaction, allergic rhinitis, Alport's syndrome, anaphylaxis, ankylosing spondylitis, anti-phospholipid syndrome, arthritis, ascariasis, aspergillosis, atopic allergy, atopic dermatitis, atopic rhinitis, Behcet's Disease, Bird fancier's lung, bronchial asthma, Caplan's Syndrome, cardiomyopathy, celiac disease, Chagas' Disease, chronic glomerulonephritis, Cogan's syndrome, cold agglutinin disease, congenital rubella infection, CREST Syndrome, Crohn's disease, cryoglobulinemia. Gushing's syndrome, dermatomyositis, discoid lupus, Dressler syndrome, Eaton-Lambert syndrome, echovirus infection, encephalomyelitis, endocrine ophthalmopathy, Epstein-Barr virus infection, equine heaves, erythematosus, Evans syndrome, Felty's syndrome, fibromyalgia, Fuchs heterochromatic iridocyclitis, gastric atrophy, gastrointestinal allergy, giant cell arteritis, glomerulonephritis, Goodpasture's syndrome, graft-versus-host disease, Graves' disease, Guillain-Barre disease, Hashimoto's thyroiditis, hemolytic anemia, Henoch-Schonlein purpura, idiopathic adrenal atrophy, idiopathic pulmonary fibrosis, IgA nephropathy, inflammatory bowel diseases, insulin-dependent diabetes mellitus, juvenile arthritis, juvenile diabetes mellitus (Type 1), Lambert-Eaton syndrome, laminitis, lichen planus, lupoid hepatitis, lupus, lymphopenia, Meniere's Disease, mixed connective tissue disease, multiple sclerosis, myasthenia gravis, pernicious anemia, polyglandular syndromes, presenile dementia, primary agammaglobulinemia, primary biliary cirrhosis, psoriasis, psoriatic arthritis, Raynaud's phenomenon, recurrent abortion, Reiter's syndrome, rheumatic fever, rheumatoid arthritis, Samter's syndrome, schistosomiasis, Schmidt's syndrome, scleroderma, Shulman's syndrome, Sjogren's syndrome, Stiff-person syndrome, sympathetic ophthalmia, systemic lupus erythematosus, Takayasu's arteritis, temporal arteritis, thyroiditis, thrombocytopenia, thyrotoxicosis, toxic epidermal necrolysis, type B insulin resistance, type I diabetes mellitus, ulcerative colitis, uveitis, vitiligo, Waldenstrom macroglobulinemia, and granulomatosis with polyangiitis.

Methods according to this disclosure may further include administering one or more drug conjugates provided herein to treat and/or prevent an autoimmune disorder in a combination therapy. For example, in certain embodiments, a combination therapy comprises administering one or more drug conjugates (concurrently or sequentially) with a therapeutic agent known to treatment and/or prevent an autoimmune disorder.

In some embodiments, the condition being treated and/or prevented is an infectious disease. In some of these embodiments, the infectious disease is a bacterial disease, systemic fungal disease, Rickettsial disease, parasitic disease, and/or viral disease.

In some embodiments, the one or more bacterial diseases include diphtheria, pertussis, occult bacteremia, urinary tract infection, gastroenteritis, cellulitis, epiglottitis, tracheitis, adenoid hypertrophy, retropharyngeal abscess, impetigo, ecthyma, pneumonia, endocarditis, septic arthritis, pneumococcal, peritonitis, bacteremia, meningitis. acute purulent meningitis, urethritis, cervicitis, proctitis, pharyngitis, salpingitis, epididymitis, gonorrhea, syphilis, listeriosis, anthrax, nocardiosis, salmonella, typhoid fever, dysentery, conjunctivitis, sinusitis, brucellosis, tularemia, cholera, bubonic plague, tetanus, necrotizing enteritis, actinomycosis, mixed anaerobic infections, syphilis, relapsing fever, leptospirosis, Lyme disease, rat bite fever, tuberculosis, lymphadenitis, leprosy, chlamydia, chlamydial pneumonia, trachoma, and/or inclusion conjunctivitis.

In some embodiments, the one or more systemic fungal diseases is selected from histoplasmosis, coccidioidomycosis, blastomycosis, sporotrichosis, cryptococcosis, systemic candidiasis, aspergillosis, mucormycosis, mycetoma, and/or chromomycosis.

In some embodiments, the one or more Rickettsial diseases is selected from typhus, Rocky Mountain spotted fever, ehrlichiosis, eastern tick-borne Rickettsioses, Rickettsialpox, Q fever, bartonellosis.

In some embodiments, the one or more parasitic diseases is selected from malaria, babesiosis, African sleeping sickness, chagas' disease, leishmaniasis, dum-dum fever, toxoplasmosis, meningoencephalitis, keratitis, amoebiasis, giardiasis, cryptosporidiosis, isosporiasis, cyclosporiasis, microsporidiosis, ascariasis, whipworm infection, hookworm infection, threadworm infection, ocular larva migrans, trichinosis, guinea worm disease, lymphatic filariasis, loiasis, river blindness, canine heartworm infection, schistosomiasis, swimmer's itch, oriental lung fluke, oriental liver fluke, fascioliasis, fasciolopsiasis, opisthorchiasis, tapeworm infections, hydatid disease, alveolar hydatid disease.

In some embodiments, the one or more viral diseases is selected from measles, subacute sclerosing panencephalitis, common cold, mumps, rubella, roseola, fifth disease, chickenpox, respiratory syncytial virus infection, croup, bronchiolitis, infectious mononucleosis, poliomyelitis, herpangina, hand-foot-and-mouth disease, Bornholm disease, genital herpes, genital warts, aseptic meningitis, myocarditis, pericarditis. gastroenteritis, acquired immunodeficiency Syndrome (AIDS), human immunodeficiency virus (HIV), Reye's syndrome, Kawasaki syndrome, influenza, bronchitis, viral “walking” pneumonia, acute febrile respiratory disease, acute pharyngoconjunctival fever, epidemic keratoconjunctivitis, herpes simplex virus 1 (hsv-1), herpes simplex virus 2 (hsv-2), shingles, cytomegalic inclusion disease, rabies, progressive multifocal leukoencephalopathy, kuru, fatal familial insomnia, Creutzfeldt-Jakob disease, Gerstraann-Straussler-Scheinker disease, tropical spastic paraparesis, western equine encephalitis, California encephalitis, St. Louis encephalitis, yellow fever, dengue, lymphocytic choriomeningitis, Lassa fever, hemorrhagic fever, hantavirus pulmonary syndrome, Marburg virus infections, Ebola virus infections, and/or smallpox.

Methods according to this disclosure may further include administering one or more drug conjugates provided herein to treat and/or prevent an infectious disease in a combination therapy. For example, in certain embodiments, a combination therapy comprises administering one or more drug conjugates (concurrently or sequentially) with a therapeutic agent known to treatment and/or prevent an infectious disease.

Non-limiting examples of compounds of the invention include:

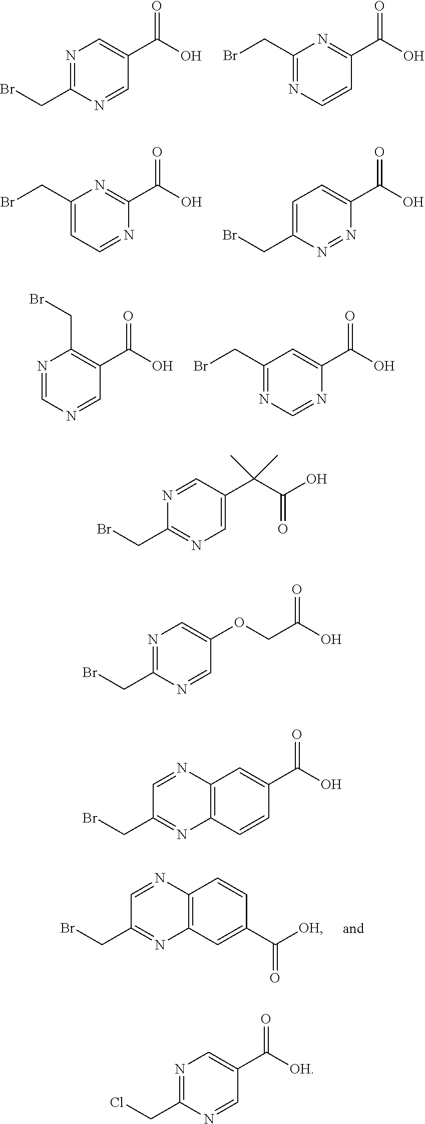

Non-limiting examples of compounds of the invention also include:

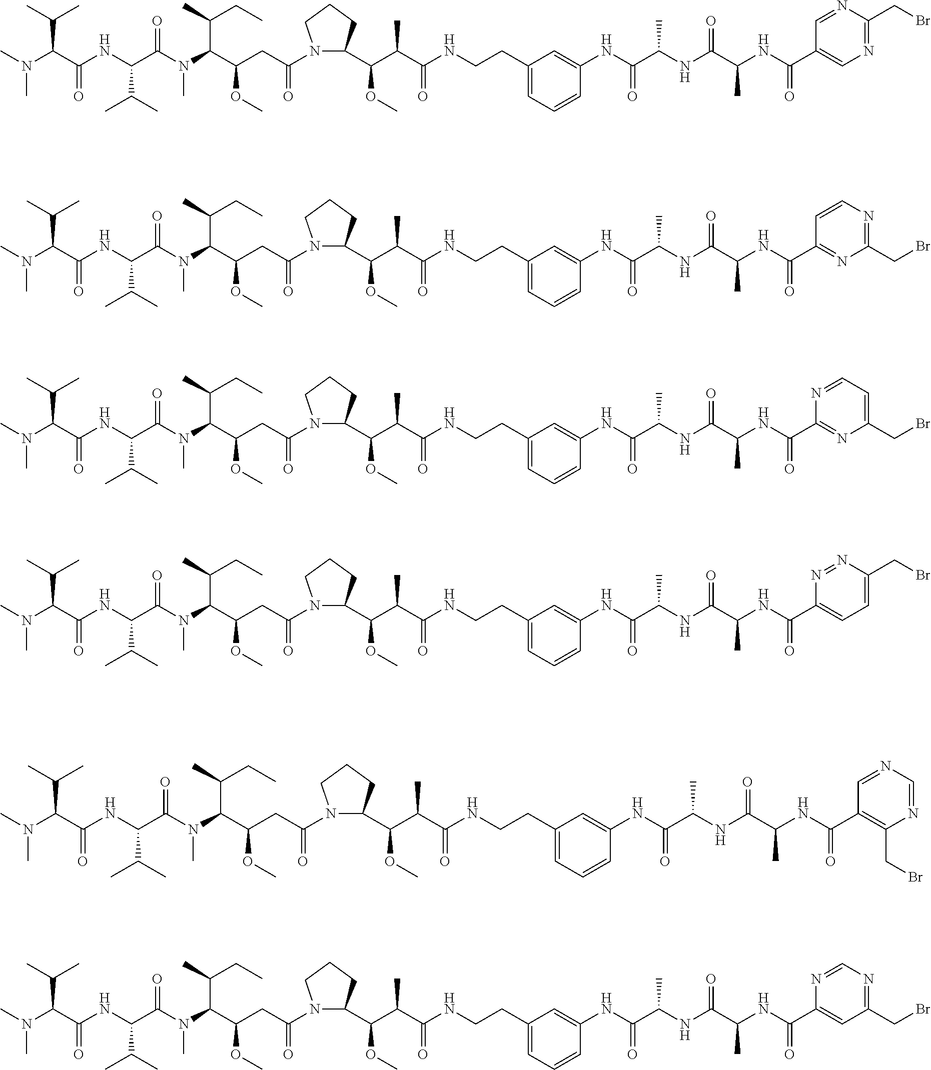

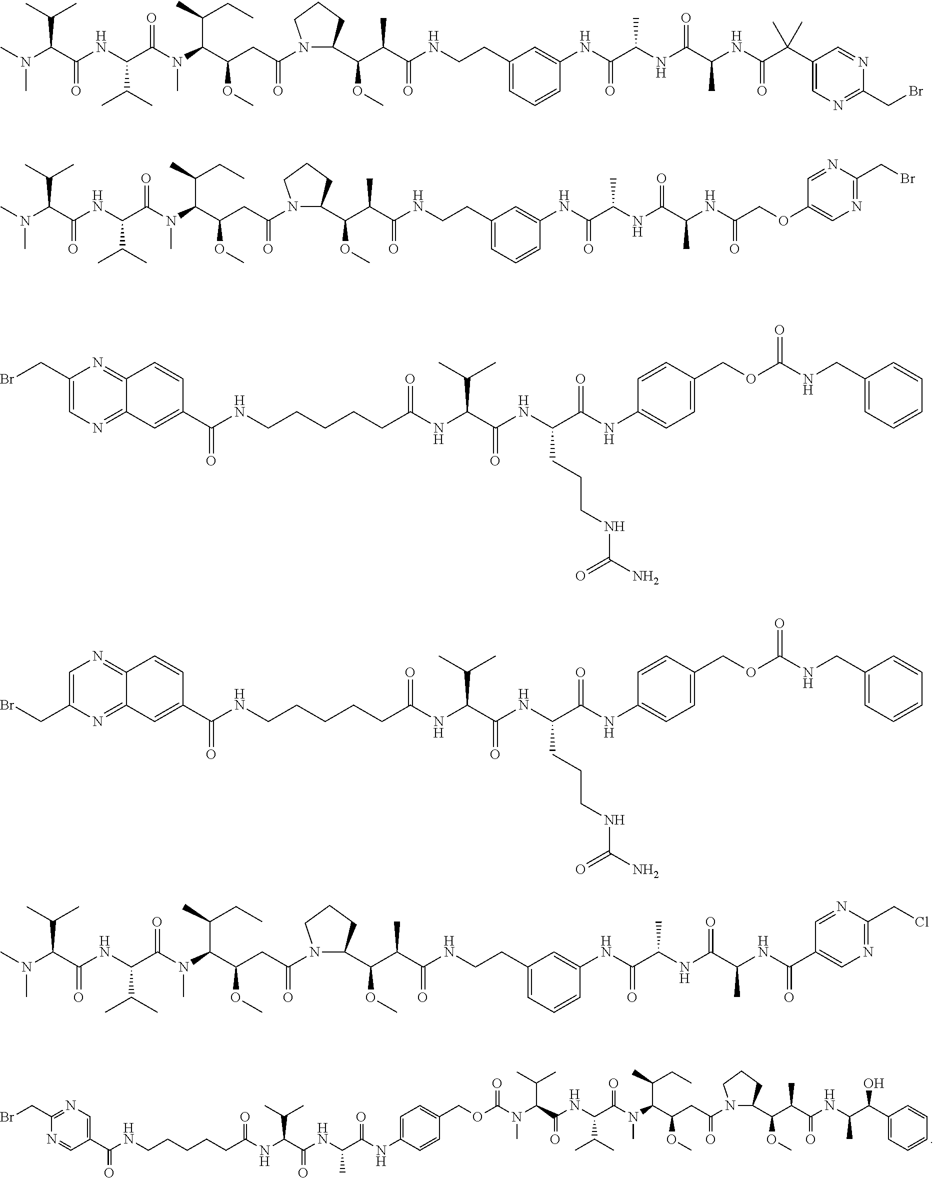

### Spacers

In certain embodiments, the spacer moiety comprises an alkyl chain. In some embodiments, the spacer moiety has the following formula: —(CH₂)ₙ, where n is 1, 2, 3, 4, 5, 6, 7, 8, 9, or 10. In some embodiments, the spacer moiety comprises a heteroalkyl chain. In some embodiments, the spacer moiety has the following formula: —(CH₂CH₂O)ₙ, where n is 1, 2, 3, 4, 5, 6, 7, 8, 9, or 10.

In some embodiments, the alkyl is a low alkyl, having 1 to 4 carbon atoms (e.g., methyl, ethyl, propyl and butyl).

In certain embodiments, the spacer moiety comprises a peptide. In certain embodiments, the peptide comprises two or more amino acids for example, a dipeptide, a tripeptide, tetrapeptide, pentapeptide, hexapeptide, heptapeptide, octapeptide, nonapeptide, or decapeptide. In some of these embodiments, the spacer moiety comprises Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, Ala-PAB, PAB, or combinations thereof.

In some embodiments, the spacer moiety comprises a combination of an alkyl, heteroalkyl, PEG, or a peptide. For example, the spacer moiety comprises —(CH₂)ₙ and a peptide, the spacer moiety comprises —(CH₂CH₂O)ₙ and a peptide, the spacer moiety comprises PEG and a peptide, the spacer moiety comprises —(CH₂)ₙ and PEG, or the spacer moiety comprises —(CH₂CH₂O)ₙ and PEG.

In some embodiments, the polypeptide moiety comprises 1 to 6 amino acids. For example, the polypeptide can include 1 amino acid, 2 amino acids, 3 amino acids, 4 amino acids, 5 amino acids, or 6 amino acids.

The polypeptide moiety may include one or more natural amino acids and/or one or more unnatural amino acids. In some embodiments, the natural amino acid is one or more of the 20 common amino acids selected from one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. As used herein, the term “unnatural amino acid” refers to any amino acid, modified amino acid, and/or amino acid analogue that is not one of the 20 common naturally occurring amino acids. Non-limiting examples of unnatural amino acids include N-acetylglucosaminyl-L-serine, N-acetylglucosaminyl-L-threonine, and O-phosphotyrosine.

### Self-Immolative Moiety

The “self-immolative moiety” refers to a chemical moiety that is capable of covalently linking two chemical moieties, for example, a polypeptide moiety and a drug moiety. The self-immolative spacer is capable of spontaneously separating from the drug moiety if the bond to the polypeptide is cleaved, e.g., via proteolytic cleavage.

In some embodiments, the self-immolative moiety is selected from:

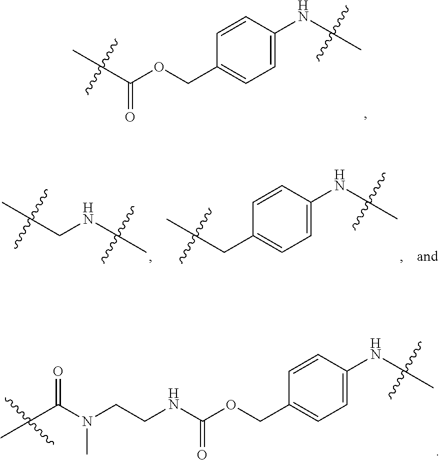

In some embodiments, a linker as provided herein is modified when conjugated to a drug moiety and/or targeting moiety, for example in a linker-drug moiety complex, linker-targeting moiety complex, or drug conjugate as provided herein. For example, where the linker comprises a hydroxyl group, that hydroxyl group may react with a functional group on the drug moiety or targeting moiety during the conjugation reaction, producing a conjugate wherein the linker no longer comprises the hydroxyl group.

### Drug Moieties

The drug moiety in the drug conjugates and components thereof provided herein may be any compound or molecule that produces a therapeutic effect, including both small molecules and biologics. By way of example, a drug moiety may be a chemical agent, such as an antibiotic, anti-cancer agent, a polypeptide, or a nucleic acid.

In some embodiments, the drug moiety is a chemotherapeutic agent, an immune modulator, a tubulin-binder, a DNA-alkylating agent, an HSP90 inhibitor, a DNA topoisomerase, an anti-epigenetic agent, an HDAC inhibitor, an anti-metabolism agent, a proteasome inhibitor, a peptide, a peptidomimetic, an siRNA, and/or an antisense DNA.

In certain embodiments, the drug is a chemotherapeutic drug. Non-limiting examples of chemotherapeutic drugs include alkylating agents, plant alkaloids, DNA topoisomerase inhibitors, anti-metabolites, hormonal therapies, kinase inhibitors, and/or antibiotics.

In some embodiments, the alkylating agent is selected from one or more of chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (e.g., adozelesin, carzelesin and bizelesin synthetic analogues); Duocarmycin (e.g., synthetic analogues, KW-2189 and CBI-TMI); Benzodiazepine dimers (e.g., dimmers of pyrrolobenzodiazepine (PBD) or tomaymycin, indolinobenzodiazepines, imidazobenzothiadiazepines, or oxazolidino-benzodiazepines), nitrosoureas (e.g., carmustine, lomustine, chlorozotocin, fotemustine, nimustine, ranimustine), alkylsulphonates (e.g., busulfan, treosulfan, improsulfan and piposulfan); triazenes (e.g., dacarbazine), platinum containing compounds (e.g., carboplatin, cisplatin, oxaliplatin), and/or aziridines (e.g., benzodopa, carboquone, meturedopa, and uredopa).

In some embodiments, the plant alkaloid is selected from one or more of vinca alkaloids (e.g., vincristine, vinblastine, vindesine, vinorelbine, navelbin), taxoids (e.g., paclitaxel and docetaxol), maytansinoids (e.g., DM1, DM2, DM3, DM4, maytansine and ansamitocins), cryptophycins (e.g., cryptophycin 1 and cryptophycin 8), epothilones, eleutherobin, discodermolide, bryostatins, dolostatins, auristatins, tubulysins, cephalostatins, pancratistatin, sarcodictyin, and/or spongistatin.

In some embodiments, the DNA topoisomerase inhibitor is selected from one or more of epipodophyllins (e.g., 9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)) and/or mitomycins (e.g., mitomycin C).

In some embodiments, the anti-metabolite is selected from one or more of anti-folate such DIFR inhibitors (e.g., methotrexate, trimetrexate, denopterin, pteropterin, aminopterin (4-aminopteroic acid) or the other folic acid analogues); IMP dehydrogenase inhibitors (e.g., mycophenolic acid, tiazofurin, ribavirin, EICAR); ribonucleotide reductase Inhibitors (e.g., hydroxyurea, deferoxamine), pyrimidine analogs such uracil analogs: (e.g., ancitabine, azacitidine, 6-azauridine, capecitabine (Xeloda), carmofur, cytarabine, dideoxyuridine, doxifluridine, enocitabine, 5-Fluorouracil, floxuridine, ratitrexed (e.g., tomudex), cytosine analogs (e.g., cytarabine, cytosine arabinoside, fludarabine), purine analogs (e.g., azathioprine, fludarabine, mercaptopurine, thiamiprine, thioguanine), and/or folic acid replenisher (e.g. frolinic acid).

In some embodiments, the hormonal therapy is one or more of receptor antagonists such anti-estrogens (e.g., megestrol, raloxifene, tamoxifen), LHRH agonists (e.g., goserelin, leuprolide), anti-androgens (e.g., bicalutamide, flutamide, calusterone, dromostanolone propionate, epitiostanol, mepitiostane, nilutamide, testolactone, trilostane and other androgens inhibitors), retinoids/deltoids (e.g., Vitamin D3 analogs: CB 1093, EB 1089 KH 1060, cholecalciferol, ergocalciferol); photodynamic therapies (e.g., verteporfin, phthalocyanine, photosensitizer Pc4, demethoxy-hypocrellin A), and cytokines (e.g., interferon-alpha, interferon-gamma, tumor necrosis factor (TNFs), human proteins containing a TNF domain).

In some embodiments, the kinase inhibitor is one or more of BIBW 2992 (e.g., anti-EGFR/Erb2), imatinib, gefitinib, pegaptanib, sorafenib, dasatinib, sunitinib, erlotinib, nilotinib, lapatinib, axitinib, pazopanib. vandetanib, E7080 (e.g., anti-VEGFR2), mubritinib, ponatinib (e.g., AP24534), bafetinib (e.g., INNO-406), bosutinib (e.g., SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, and/or ispinesib.

In some embodiments, the antibiotic is an enediyne antibiotic (e.g., calicheamicins, especially calicheamicin γ1, δ1, α1 and β1), dynemicin (e.g., dynemicin A and deoxydynemicin; esperamicin, kedarcidin, C-1027, maduropeptin, as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and/or zorubicin.

In some embodiments, the drug is an anti-autoimmune disease drug. Non-liming examples of anti-autoimmune disease drugs include cyclosporine, cyclosporine A, aminocaproic acid, azathioprine, bromocriptine, chlorambucil, chloroquine, cyclophosphamide, corticosteroids (e.g., amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, and tacrolimus.

In some embodiments, the anti-autoimmune disease drug is selected from one or more of polyketides (e.g., acetogenins such bullatacin and bullatacinone), gemcitabine, epoxomicins (e. g. carfilzomib), bortezomib, thalidomide, lenalidomide, pomalidomide, tosedostat, zybrestat, PLX4032, STA-9090, stimuvax, allovectin-7, xegeva, provenge, yervoy, isoprenylation inhibitors (e.g., Lovastatin), dopaminergic neurotoxins (e.g., 1-methyl-4-phenylpyridinium ion), cell cycle inhibitors (e.g., staurosporine), actinomycins (e.g., actinomycin D, dactinomycin), bleomycins (e.g., bleomycin A2, bleomycin B2, peplomycin), anthracyclines (e.g., daunorubicin, doxorubicin, idarubicin, epirubicin, pirarubicin, zorubicin, mitoxantrone, MDR inhibitors), Ca²⁺ ATPase inhibitors (e.g., thapsigargin), histone deacetylase inhibitors (e.g., Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A), thapsigargin, celecoxib, glitazones, epigallocatechin gallate, disulfiram, salinosporamide A., anti-adrenals, urethane, siRNA, antisense drugs, and/or a nucleolytic enzyme.

In certain embodiments, the drug is an infectious disease drug. Non-limiting examples of infectious disease drugs include aminoglycosides, amphenicols, ansamycins, carbapenems, cephems, glycopeptides, glycylcyclines, β-lactamase inhibitors. lincosamides, lipopeptides, macrolides, monobactams, oxazolidinones, penicillin, polypeptides, quinolones, streptogramins, sulfonamides, steroid antibacterials, tetracyclines, and/or antibiotics.

In some embodiments, the aminoglycoside is one or more of amikacin, astromicin, gentamicin (e.g., netilmicin, sisomicin, and isepamicin), hygromycin B, kanamycin (e.g., amikacin, arbekacin, bekanamycin, dibekacin, and tobramycin), neomycin (e.g., framycetin, paromomycin, and ribostamycin), netilmicin, spectinomycin, streptomycin, tobramycin, and/or verdamicin.

In some embodiments, the amphenicol is one or more of azidamfenicol, chloramphenicol, florfenicol, and/or thiamphenicol.

In some embodiments, ansamycin is one or more of geldanamycin and/or herbimycin.

In some embodiments, carbapenems is one more of biapenem, doripenem, ertapenem, imipenem/cilastatin, meropenem, and/or panipenem.

In some embodiments, the cephem is one or more of carbacephem (e.g., loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (e.g., cefoxitin, cefotetan, and cefmetazole), and/or oxacephem (e.g., flomoxef and latamoxef).

In some embodiments, the glycopeptide is one or more of bleomycin, vancomycin (oritavancin, telavancin), teicoplanin (dalbavancin), ramoplanin.

In some embodiments, the glycylcyclines is tigecycline.

In some embodiments, the β-Lactamase inhibitor is one or more of a penam (e.g., sulbactam and tazobactam) and/or a clavam (e.g., clavulanic acid).

In some embodiments, the lincosamide is one or more of clindamycin and/or lincomycin.

In some embodiments, the lipopeptide is one or more of daptomycin, A54145, and/or calcium-dependent antibiotics (CDA).

In some embodiments, the macrolide is one or more of azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, and/or telithromycin.

In some embodiments, the monobactams is selected from aztreonam and/or tigemonam.

In some embodiments, the oxazolidinones is linezolid.

In some embodiments, the penicillin is one or more of amoxicillin, ampicillin (e.g., pivampicillin, hetacillin, bacampicillin, metampicillin, talampicillin), azidocillin, azlocillin, benzylpenicillin, benzathine benzylpenicillin, benzathine phenoxymethylpenicillin, clometocillin, procaine benzylpenicillin, carbenicillin (e.g., carindacillin), cloxacillin, dicloxacillin, epicillin, flucloxacillin, mecillinam (e.g., pivmecillinam), mezlocillin, meticillin, nafcillin, oxacillin, penamecillin, penicillin, pheneticillin, phenoxymethylpenicillin, piperacillin, propicillin, sulbenicillin, temocillin, and/or ticarcillin.

In some embodiments, the polypeptide is one or more of bacitracin, colistin, and/or polymyxin B.

In some embodiments, the quinolone is selected from one or more of alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, and/or trovafloxacin.

In some embodiments, streptogramins is pristinamycin such as quinupristin and/or dalfopristin.

In some embodiments, the sulfonamide is one or more of mafenide, prontosil, sulfacetamide, sulfamethizole, sulfanilimide, sulfasalazine, sulfisoxazole, trimethoprim, and/or trimethoprimsulfamethoxazole (co-trimoxazole).

In some embodiments, the steroid antibacterial is fusidic acid.

In some embodiments, the tetracyclines is one or more of doxycycline, chlortetracycline, clomocycline, demeclocycline, lymecycline, meclocydine, metacycline, minocycline, oxytetracycline, penimepicycline, rolitetracycline, tetracydine, and/or glycylcyclines (e.g., tigecycline).

In some embodiments, the anti-infectious disease drug is an antibiotic selected from one or more of annonacin, arsphenamine, bactoprenol inhibitors (e.g., bacitracin), DADAL/AR inhibitors (e.g., cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g., fosfomycin), nitrofurantoin, paclitaxel, platensimycin, pyrazinamide, quinupristin/dalfopristin, rifampicin (e.g., rifampin), tazobactam tinidazole, and/or uvaricin.

### Targeting Moieties

The targeting moiety in the drug conjugates and components thereof provided herein may be any compound or molecule capable of specifically binding to a target. By way of example, a targeting moiety may be a small molecule, a peptide, a polypeptide, or a nucleic acid such as an aptamer.

In certain embodiments, the targeting moiety is a polypeptide, for example a protein ligand, protein scaffold, or antibody. In certain embodiments, the targeting moiety is a monoclonal antibody.

In some embodiments, the targeting moiety comprises HuM195-Ac-225, HuM195-Bi-213, Anyara (naptumomab estafenatox; ABR-217620), AS 1409, Zevalin (ibritumomab tiuxetan), BIIB015, BT-062, Neuradiab, CDX-1307, CR011-vcMMAE, Trastuzumab-DMl (R3502), Bexxar (tositumomab), IMGN242, IMGN388, IMGN901, ¹³¹I-labetuzumab, IMMU-102 (⁹⁰Y-epratuzumab), IMMU-107 (⁹⁰Y-clivatuzumab tetraxetan), MDX-1203, CAT-8015, EMD 273063 (hul4.18-IL2), Tucotuzumab celmoleukin (EMD 273066; huKS-IL2), ¹⁸⁸Re-PTI-6D2, Cotara, L19-IL2, Teleukin (F16-IL2), Tenarad (F16-¹³¹I), L19-¹³¹I, L19-TNF, PSMA-ADC, DI-Leul6-IL2, SAR3419, SGN-35, and/or CMC544, or a target-binding portion thereof.

In some embodiments, the targeting moiety comprises Brentuximab vedotin, Trastuzumab emtansine, Inotuzumab ozogamicin, Lorvotuzumab mertansine, Glembatumumab vedotin, SAR3419, Moxetumomab pasudotox, AGS-16M8F, BIIB-015, BT-062, and/or IMGN-388, or a target-binding portion thereof.

### Kits

Provided herein in certain embodiments are kits comprising one or more of the drug conjugates or components thereof provided herein. In certain embodiments, the kits further comprise instructions for use.

In some embodiments, the kits provided herein are for use in preparing a drug conjugate as disclosed herein. For example, the kit may comprise one or more of a linker, a drug moiety, and a targeting moiety, and may further comprise instructions for using the provided components to generate a drug conjugate.

In some embodiments, the kits provided herein are for use in a method of treatment as disclosed herein. For example, the kit may comprise a drug conjugate or all of the components of a drug conjugate, and may further comprise instructions for preparing and/or administering the drug conjugate.

As can be appreciated from the disclosure above, the present invention has a wide variety of applications. The invention is further illustrated by the following examples, which are only illustrative and are not intended to limit the definition and scope of the invention in any way.

### Examples

### Synthesis

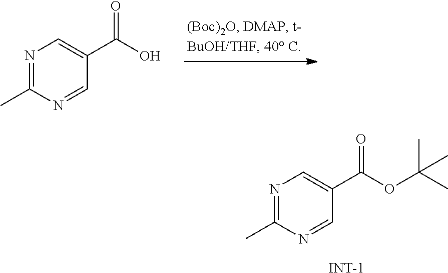

### Tert-butyl 2-methylpyrimidine-5-carboxylate INT-1

To a solution of 2-methylpyrimidine-5-carboxylic acid (1.50 g, 10.87 mmol) and DMAP (1.33 g, 10.87 mmol) in t-BuOH/THF (50 mL, 1:1) was added Boc₂O (3.56 g, 16.31 mmol) in one charge. The resulting mixture was stirred at 40° C. for 15 h under nitrogen. It was diluted with H₂O (50 mL), extracted with EA (50 mL*3). The organic layers were combined, washed with brine (50 mL), dried over Na2SO4, filtrated and concentrated. The residue was purified by Combi-Flash (PE/EA= 1/10) to give INT-1 (1.80 g, 85%) as a yellow oil. LCMS(ESI): m/z=195.1 [M+H]⁺.

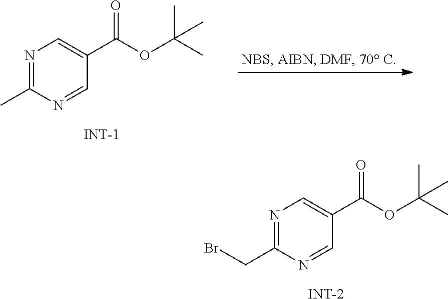

### Tert-butyl 2-(bromomethyl)pyrimidine-5-carboxylate INT-2

To a solution of INT-1 (1.80 g, 9.28 mmol) in DMF (40 mL) was added NBS (2.14 g, 12.06 mmol) and AIBN (1.52 g, 9.28 mmol). The resulting mixture was stirred at 70° C. for 6 h. The mixture was diluted with EA (60 mL), washed with deionized water (50 mL*3), dried over anhydrous Na2SO4, filtrated and concentrated. The residue was purified by Flash Chromatography (0-10%, PE/EA) to give INT-2 (1.00 g, 39%) as a yellow oil. LCMS(ESI): m/z=273.0/275.0 [M+H]⁺.

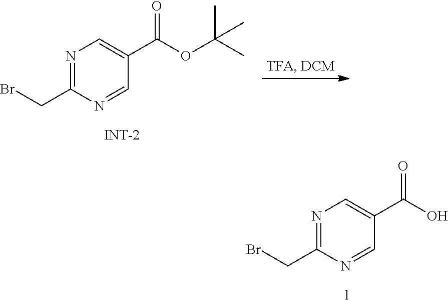

### 2-(Bromomethyl)pyrimidine-5-carboxylic acid 1

To a solution of INT-2 (1.00 g, 3.66 mmol) in DCM (10 mL) was added TFA (4 mL) dropwise. The resulting mixture was stirred at 25° C. for 8 h. The solvent was removed off under reduced pressure and the residue was lyophilized to give 1 (650 mg, 82%) as a white solid. LCMS(ESI): m/z=215.0/217.0 [M+H]⁺. NMR (400 MHz, DMSO) δ 9.23 (s, 2H), 4.76 (s, 2H).

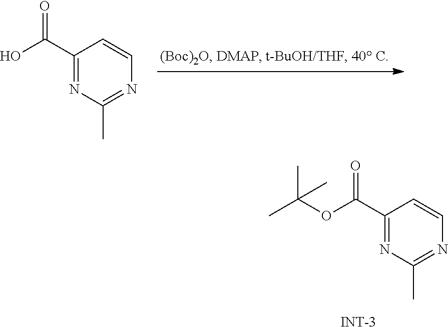

### Tert-butyl 2-methylpyrimidine-4-carboxylate INT-3

To a solution of 2-methylpyrimidine-4-carboxylic acid (2.00 g, 14.39 mmol) and DMAP (1.76 g, 14.39 mmol) in t-BuOH/THF (60 mL, 1:1) was added (Boc)₂O (4.71 g, 21.59 mmol) in one portion. The mixture was stirred at 40° C. for 16 h under nitrogen, diluted with H₂O (50 mL), extracted with EtOAc (50 mL*3). The organic layers were combined, washed with H₂O (50 mL*2), dried over Na₂SO₄, filtered and concentrated. The residue was purified by Combi-Flash (petroleum ether: EtOAc= 1/10) to give INT-3 (2.10 g, 75%) as a yellow oil. LCMS (ESI): m/z 195.1 [M+H]⁺.

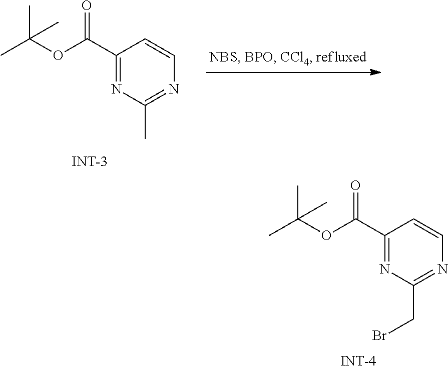

### Tert-butyl 2-(bromomethyl)pyrimidine-4-carboxylate INT-4

To a solution of INT-3 (1.50 g, 7.69 mmol) in CCl₄ (40 mL) was added NBS (2.14 g, 12.06 mmol) and BPO (1.52 g, 9.28 mmol) at 70° C. The resulting mixture was stirred at 78° C. for 6 h, diluted with EA (60 mL), washed with deionized water (50 mL*3), dried over anhydrous Na2SO4, filtered and concentrated. The residue was purified by Flash Chromatography (0-15%, petroleum ether: EtOAc) to give INT-4 (0.6 g, 29%) as a yellow oil. LCMS(ESI): m/z 216.9 [M-55]⁺.

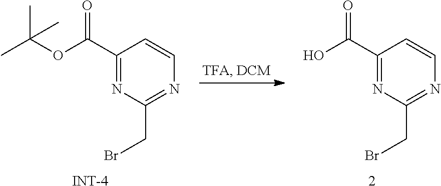

### 2-(Bromomethyl)pyrimidine-4-carboxylic acid 2

To a solution of INT-4 (0.30 g, 1.09 mmol) in DCM (3 mL) was added TFA (1 mL). The resulting mixture was stirred at 25° C. for 8 h. The solvent was removed off under reduced pressure and the residue was lyophilized to give 2 (240 mg) as a yellow solid. LCMS(ESI): m/z 217.0 [M+H]⁺. NMR (400 MHz, CDCl₃) δ 8.95 (d, J=5.0 Hz, 1H), 7.77 (d, J=5.0 Hz, 1H), 4.70 (s, 2H), 1.63(S, 9H).

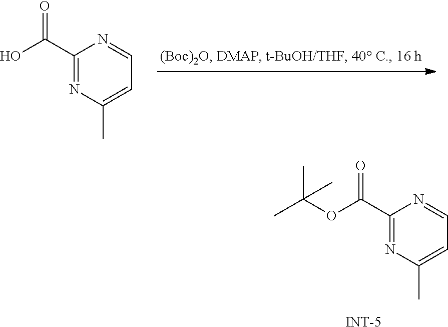

### Tert-butyl 4-methylpyrimidine-2-carboxylate INT-5

To a solution of 4-methylpyrimidine-2-carboxylic acid (1.00 g, 7.19 mmol) and DMAP (0.88 g, 7.19 mmol) in t-BuOH/THF (30 mL, 1:1) was added (Boc)₂O (2.35 g, 10.79 mmol) in one portion. The mixture was stirred at 40° C. for 15 h under nitrogen. It was diluted with H₂O (50 mL), extracted with EtOAc (50 mL*3). The organic layers were combined, washed with H₂O (50 mL*2), dried over Na2SO4, filtrated and concentrated. The residue was purified by Combi-Flash (petroleum ether: EtOAc= 1/10) to give INT-5 (1.30 g, 93%) as a yellow oil. LCMS(ESI): m/z 411.2 [2M+Na]⁺.

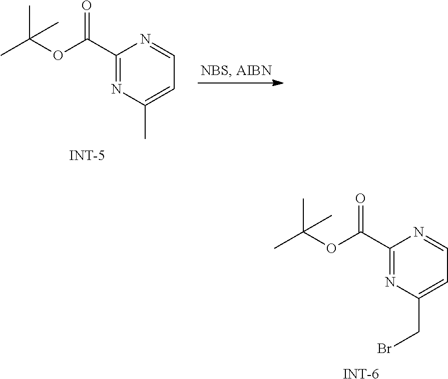

### Tert-butyl 4-(bromomethyl)pyrimidine-2-carboxylate INT-6

To a solution of INT-5 (1.20 g, 6.15 mmol) in DMF (40 mL) was added NBS (1.42 g, 7.99 mmol) and AIBN (1.01 g, 6.15 mmol). The resulting mixture was stirred at 70° C. for 4 h. The reaction mixture was diluted with EA (60 mL), washed with deionized water (50 mL*3), dried over anhydrous Na₂SO₄, filtrated and concentrated. The residue was purified by Flash Chromatography (0-10%, petroleum ether: EtOAc) to give INT-6 (0.14 g, 8%) as a yellow solid. LCMS(ESI): m/z 217.0 [M −55]⁺.

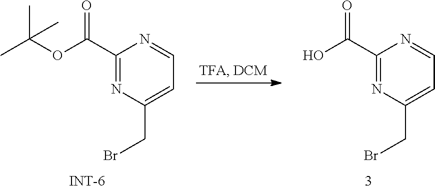

### 4-(Bromomethyl)pyrimidine-2-carboxylic acid 3

To a solution of INT-6 (0.10 g, 0.36 mmol) in DCM (1.2 mL) was added TFA (0.4 mL). The resulting mixture was stirred at 25° C. for 8 h. The solvent was removed off under reduced pressure and the residue was lyophilized to give 3 (80 mg) as a yellow solid. LCMS(ESI): m/z 217.0 [M+H]⁺. ¹H NMR (400 MHz, CDCl₃) δ 8.91 (d, J=5.1 Hz, 1H), 7.65 (d, J=5.1 Hz, 1H), 4.56 (s, 2H), 1.67 (s, 9H).

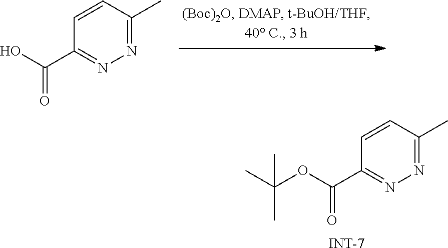

### Tert-butyl 6-methylpyridazine-3-carboxylate INT-7

To a solution of 6-methylpyridazine-3-carboxylic acid (1.0 g, 7.2 mmol) and DMAP (2.36 g, 10.8 mmol) in t-BuOH/THF (50 mL, 1:1) was added Boc2O (970 mg, 7.9 mmol) in one portion. The mixture was stirred at 40° C. for 15 h under nitrogen. It was diluted with H₂O (50 mL), extracted with EtOAc (50 mL*3). The organic layers were combined, washed with brine (50 mL), dried over Na2SO4, filtrated and concentrated. The residue was purified by Combi-Flash (petroleum ether: EtOAc= 1/10) to give INT-7 (1.2 g, 85%) as a yellow oil. LCMS (ESI): m/z 195.1 [M+H]⁺.

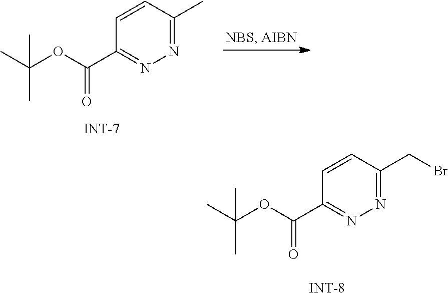

### Tert-butyl 6-(bromomethyl)pyridazine-3-carboxylate INT-8

To a solution of INT-7 (500 mg, 2.56 mmol) in DMF (20 mL) was added NBS (589 mg, 3.32 mmol) and AIBN (420 mg, 2.56 mmol). The resulting mixture was stirred at 70° C. for 6 h. The solvent was diluted with EtOAc (60 mL), washed with deionized water (50 mL*3), dried over anhydrous Na2SO4, filtered and concentrated. The residue was purified by Flash Chromatography (0-10%, petroleum ether: EtOAc) to give INT-8 (250 mg, 36%) as a yellow oil. ¹H NMR (400 MHz, CDCl3) δ 8.18 (d, J=8.7 Hz, 1H), 7.84 (d, J=8.7 Hz, 1H), 4.85 (s, 2H), 1.67 (s, 9H).

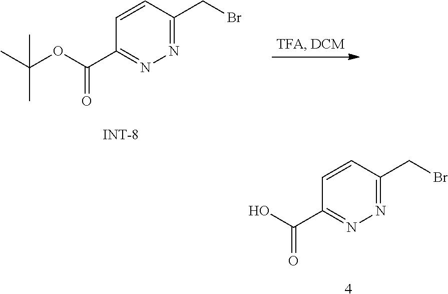

### 6-(Bromomethyl)pyridazine-3-carboxylic acid 4

To a solution of INT-8 (100 mg, 0.36 mmol) in DCM (10 mL) was added TFA (4 mL). The resulting mixture was stirred at 25° C. for 8 h. The solvent was removed off under reduced pressure and the residue was lyophilized to give 4 (80 mg) as a purple solid. LCMS(ESI): m/z 217.0 [M+H]⁺. ¹H NMR (400 MHz, DMSO) δ 8.23 (d, J=8.26 Hz, 1H), 8.03 (d, J=8.7 Hz, 1H), 4.99 (s, 2H).

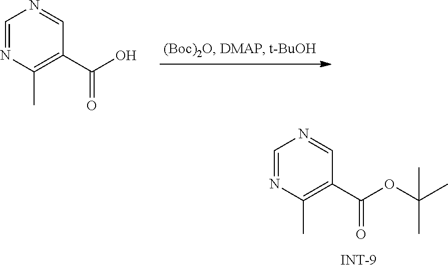

### Tert-butyl 4-methylpyrimidine-5-carboxylate INT-9

To a solution of 4-methylpyrimidine-5-carboxylic acid (1.70 g, 12.23 mmol) and DMAP (1.49 g, 12.23 mmol) in t-BuOH/THF (50 mL, 1:1) was added (Boc)₂O (4.00 g, 18.35 mmol) in one portion. The mixture was stirred at 40° C. for 15 h under nitrogen, diluted with H₂O (60 mL), extracted with EtOAc (50 mL*3). The organic layers were combined, washed with H₂O (50 mL*2), dried over Na2SO4, filtrated and concentrated. The residue was purified by Combi-Flash (petroleum ether: EtOAc= 1/10) to give INT-9 (2.20 g, 92%) as a yellow oil. LCMS (ESI): m/z 195.2 [M+H]⁺.

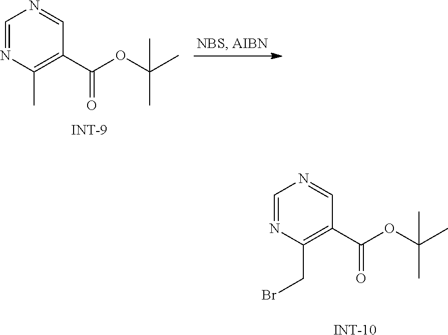

### Tert-butyl 4-(bromomethyl)pyrimidine-5-carboxylate INT-10

To a solution of INT-9 (2.20 g, 11.28 mmol) in DMF (60 mL) was added NBS (2.60 g, 14.66 mmol) and AIBN (1.85 g, 11.28 mmol). The resulting mixture was stirred at 70° C. for 6 h, diluted with EtOAc (60 mL), washed with deionized water (50 mL*3), dried over anhydrous Na2SO4, filtered and concentrated. The residue was purified by Flash Chromatography (0-20%, petroleum ether: EtOAc) to give INT-10 (0.50 g, 16%) as a yellow oil. LCMS(ESI): m/z 273.0 [M+H]⁺.

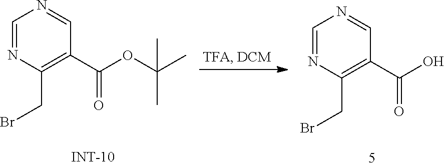

### 4-(Bromomethyl)pyrimidine-5-carboxylic acid 5

To a solution of INT-10 (0.50 g, 1.83 mmol) in DCM (3 mL) was added TFA (1 mL). The resulting mixture was stirred at 25° C. for 8 h. The solvent was removed off under reduced pressure and the residue was lyophilized to give 5 (400 mg) as a white solid. LCMS(ESI): m/z 217.0 [M+H]⁺. ¹H NMR (400 MHz, CDCl₃) δ 9.22 (s, 1H), 9.17 (s, 1H), 4.87 (s, 2H), 1.63 (s, 9H).

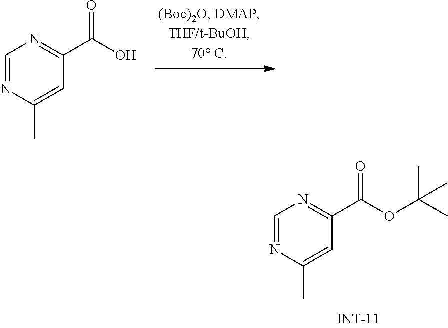

### Tert-butyl 6-methylpyrimidine-4-carboxylate INT-11

To a solution of 6-methylpyrimidine-4-carboxylic acid (1.60 g, 11.51 mmol) and DMAP (1.40 g, 11.51 mmol) in t-BuOH/THF (50 mL, 1:1) was added (Boc)₂O (3.76 g, 17.27 mmol) in one portion. The mixture was stirred at 70° C. for 30 h under nitrogen, diluted with H₂O (50 mL), extracted with EtOAc (50 mL*3). The organic layers were combined, washed with H₂O (50 mL*2), dried over Na₂SO₄, filtered and concentrated. The residue was purified by Combi-Flash (petroleum ether: EtOAc= 1/10) to give INT-11 (1.0 g, 45%) as a yellow oil. LCMS (ESI): m/z 195.1 [M+H]⁺.

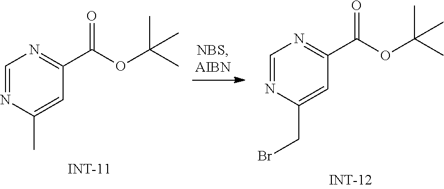

### Tert-butyl 6-(bromomethyl)pyrimidine-4-carboxylate INT-12

To a solution of INT-11 (0.70 g, 3.59 mmol) in DMF (20 mL) was added NBS (0.83 g, 4.67 mmol) and AIBN (0.59 g, 3.59 mmol). The resulting mixture was stirred at 70° C. for 6 h, diluted with EtOAc (60 mL), washed with deionized water (50 mL*3), dried over anhydrous Na2SO4, filtered and concentrated. The residue was purified by Flash Chromatography (0-10%, petroleum ether: EtOAc) to give INT-12 (0.33 g, 34%) as a yellow oil. LCMS(ESI): m/z 217.0 [M−55]⁺.

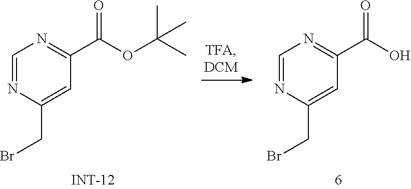

### 6-(Bromomethyl)pyrimidine-4-carboxylic acid 6

To a solution of INT-12 (0.30 g, 1.10 mmol) in DCM (3 mL) was added TFA (1 mL). The resulting mixture was stirred at 25° C. for 8 h. The solvent was removed off under reduced pressure and the residue was lyophilized to give 6 (240 mg) as a white solid. LCMS(ESI): m/z 217.0 [M+H]⁺. ¹H NMR (400 MHz, CDCl₃) δ 9.33 (d, J=1.0 Hz, 1H), 8.07 (d, J=1.1 Hz, 1H), 4.52 (s, 2H), 1.65 (S, 9H).

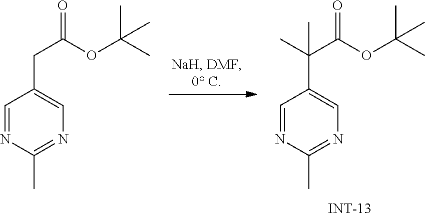 [SMILES: Cc1ncc(CC(=O)OC(C)(C)C)cn1 ; Cc1ncc(C(C)(C)C(=O)OC(C)(C)C)cn1]

### Tert-butyl 2-methyl-2-(2-methylpyrimidin-5-yl)propanoate INT-13

To a solution of tert-butyl 2-(2-methylpyrimidin-5-yl)acetate (500 mg, 2.4 mmol) and sodium hydride (384 mg, 9.6 mmol, 60% in mineral oil) in DMF (2.5 mL) stirred under nitrogen at 0° C. was added a solution of iodomethane (716 mg, 5.0 mmol) in DMF (0.5 mL). The reaction mixture was stirred at 25° C. for 1 h. Diluting with water (15 mL), extracted with EtOAc (3*10 mL), washed with brine, and the organic phase was dried over sodium sulphate, filtrated, and evaporated under vacuum. The crude product was purified by Flash Chromatography (petroleum ether: EtOAc=5:1) to give INT-13 (220 mg, 33.89%) as a yellow oil. LCMS(ESI): m/z 237.2 [M+H]⁺.

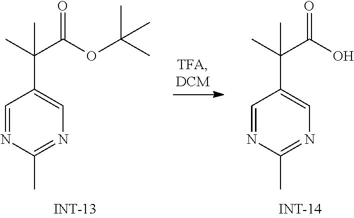 [SMILES: Cc1ncc(C(C)(C)C(=O)OC(C)(C)C)cn1 ; Cc1ncc(C(C)(C)C(=O)O)cn1]

### 2-Methyl-2-(2-methylpyrimidin-5-yl)propanoic acid INT-14

To a solution of INT-13 (100 mg, 0.3 mmol) in DCM (3 mL) stirred at 25° C. was added TFA (1 mL). The reaction mixture was stirred at 25° C. for 3 h, evaporated under vacuum, to give INT-14 (80 mg, crude) as a yellow solid. LCMS(ESI): m/z 181.2 [M+H]⁺.

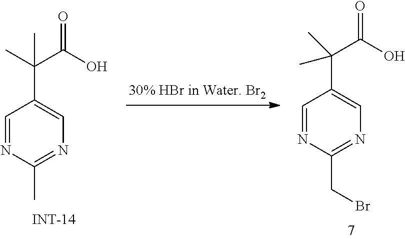

### 2-(2-(Bromomethyl)pyrimidin-5-yl)-2-methylpropanoic acid 7

To a solution of INT-14 (80 mg, 0.44 mmol) in HBr(30% in water, 2 mL) stirred at 25° C. was added a solution of Br₂ (35 mg, 0.22 mmol) in AcOH (0.2 mL). The reaction mixture was stirred at 50° C. for 16 h. Evaporated under vacuum, purified by flash C18 ODS (ACN-H20 30%) to give 7 (50 mg, 41.29%) as a brown solid. LCMS(ESI): m/z 258.9 [M+H]⁺.

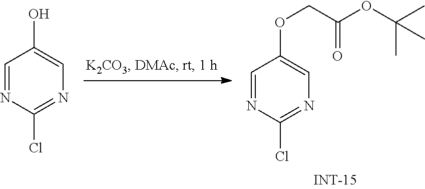 [SMILES: Oc1cnc(Cl)nc1 ; CC(C)(C)OC(=O)COc1cnc(Cl)nc1]

### Tert-butyl 2-((2-chloropyrimidin-5-yl)oxy)acetate INT-15

To a solution of 2-chloropyrimidin-5-ol (1300 mg, 9.96 mmol) and tert-butyl 2-bromoacetate (1943 mg, 9.96 mmol) in DMAc (15 mL) was added K₂CO₃ (2065 mg, 14.94 mmol) to stirred at 25° C. for 1.5 h. The mixture was diluted with H2O (40 mL), extracted with EtOAc (20 mL*3). The combined organic layers were washed with H₂O and brine, dried over Na2SO4, filtered and concentrated to afford INT-15 as a white solid (2200 mg, 83%). LCMS(ESI): m z 245.1 (M+H)+. ¹H NMR (400 MHz, CDCl3) δ 8.26 (s, 2H), 4.58 (s, 2H), 1.46 (s, 9H).

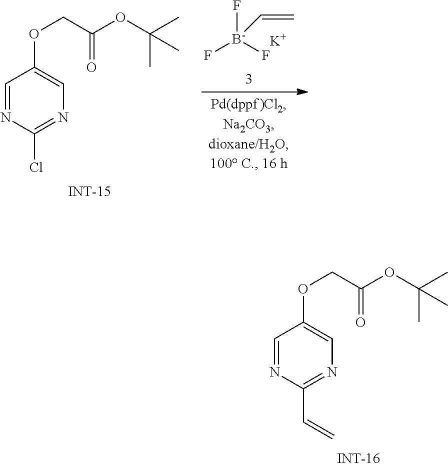

### Tert-butyl 2-((2-vinylpyrimidin-5-yl)oxy)acetate INT-16

To a mixture of INT-15 (2000 mg, 8.17 mmol), potassium ethenyltrifluoroboranuide (1095 mg, 8.17 mmol) and Pd(dppf)C12 (300 mg, 0.41 mmol) in 1,4-dioxane/H₂O(4:1, 25 mL) was added sodium carbonate (1733 mg, 16.35 mmol) to stirred at 100° C. for 4 h. The mixture was cooled to room temperature, diluted with H2O (30 mL), extracted with EA (30 mL*3). The combined organic layers were washed with H₂O and brine, dried over Na₂SO₄, filtered and concentrated. The residue was purified by Flash Chromatography (petroleum ether: EtOAc=3:1, v/v) to afford INT-16 as a white solid (1000 mg, 48%). LCMS(ESI): m/z 237.1 (M+H)+. ¹H NMR (400 MHz, CDCl₃) δ 8.40 (s, 2H), 6.90 (dd, J=17.3, 10.7 Hz, 1H), 6.52 (dd, J=17.3, 1.5 Hz, 1H), 5.69 (dd, J=10.7, 1.5 Hz, 1H), 4.61 (s, 2H), 1.50 (s, 9H).

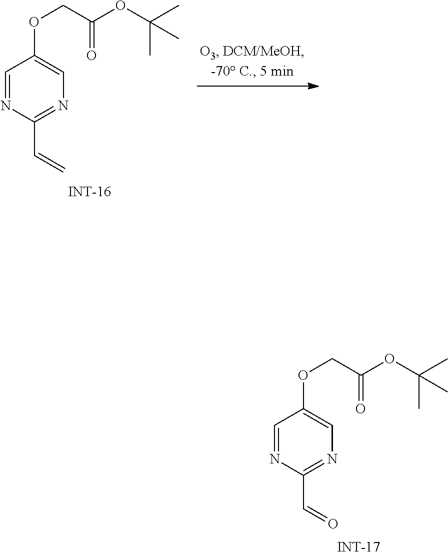 [SMILES: C=Cc1ncc(OCC(=O)OC(C)(C)C)cn1 ; CC(C)(C)OC(=O)COc1cnc(C=O)nc1]

### Tert-butyl 2-((2-formylpyrimidin-5-yl)oxy)acetate INT-17

A solution of INT-16 (1000 mg, 4.2325 mmol) in DCM/MeOH (30 mL) was cooled to −78° C., then bubbled into O₃ until the solution was blue and was stirred for 5 min. The mixture was quenched with Me2S, then the mixture was diluted with 40 mL H₂O, extracted with DCM (40 mL*3). The combined organic layers were washed with H₂O and brine, dried over Na₂SO₄, filtered and concentrated. The residue was purified by Flash Chromatography (petroleum ether: EtOAc=2:1, v/v) to afford INT-17 as a white solid (360 mg, 30%). LCMS (ESI): m z 239.1 (M+H). ¹H NMR (400 MHz, CDCl3) δ 10.04 (s, 1H), 8.57 (s, 2H), 4.72 (s, 2H), 1.50 (s, 9H).

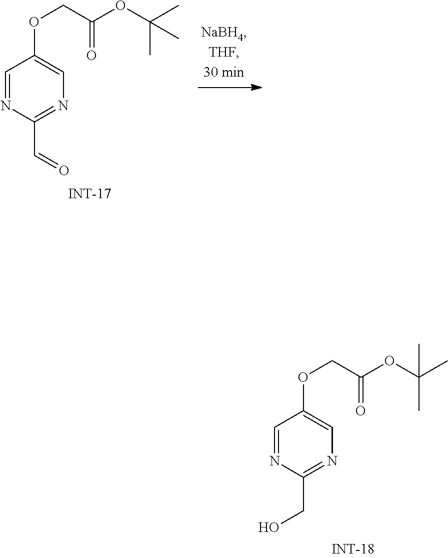 [SMILES: CC(C)(C)OC(=O)COc1cnc(C=O)nc1 ; CC(C)(C)OC(=O)COc1cnc(CO)nc1]

### Tert-butyl 2-((2-(hydroxymethyl)pyrimidin-5-yl)oxy)acetate INT-18

To a solution of INT-17 (360 mg, 1.51 mmol) in THF (5 mL) was added Sodium borohydride(86 mg, 2.26 mmol). The reaction was stirred at 25° C. for 30 min. The mixture was diluted with H2O (10 mL), extracted with DCM (10 mL*3). The combined organic layers were washed with H₂O and brine, dried over Na₂SO₄, filtered and concentrated. The residue was purified by Flash Chromatography (petroleum ether: EtOAc=1:1, v/v) to afford INT-18 as a white solid (200 mg, 50%). LCMS (ESI): m z 241.0 (M+H)⁻. ¹H NMR (400 MHz, CDCl3) δ 8.39 (s, 2H), 4.79 (s, 2H), 4.60 (s, 2H), 1.49 (s, 9H).

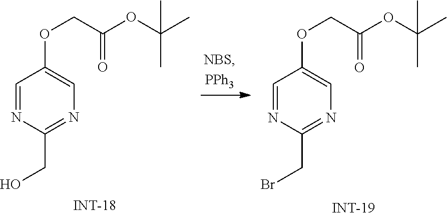 [SMILES: CC(C)(C)OC(=O)COc1cnc(CO)nc1 ; CC(C)(C)OC(=O)COc1cnc(CBr)nc1]

### Tert-butyl 2-((2-(bromomethyl)pyrimidin-5-yl)oxy)acetate INT-19

To a solution of INT-18 (100 mg, 0.42 mmol) and triphenylphosphine (218 mg, 0.83 mmol) in dry DCM (2 mL) was added NBS (148 mg, 0.83 mmol) at 0° C. The mixture was stirred at 25° C. for 1.5 h. The mixture was diluted with H2O (10 mL), extracted with DCM (10 mL*3). The combined organic layers were washed with H₂O and brine, dried over Na₂SO₄, filtrated and concentrated to dry. The residue was purified by Flash Chromatography (petroleum ether: EtOAc=1:1, v/v) to afford INT-19 as a white solid (100 mg, 79%). LCMS (ESI): m z 241.0 (M+H)+. ¹H NMR (400 MHz, CDCl3) δ 8.40 (s, 2H), 4.62 (brs, 4H), 1.50 (s, 9H).

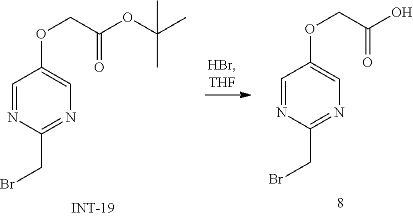 [SMILES: CC(C)(C)OC(=O)COc1cnc(CBr)nc1 ; O=C(O)COc1cnc(CBr)nc1]

### 2-((2-(Bromomethyl)pyrimidin-5-yl)oxy)acetic acid 8

To a solution of INT-19 (50 mg, 0.1656 mmol) in dry THF(2 mL) was added HBr (1 mL) at 0° C. under nitrogen. The mixture was stirred at 25° C. for 1.5 h. THF was removed off under reduced pressure, the residue was lyophilized to afford 8 as a yellow solid (32 mg, 80%). LCMS (ESI): m z 247.0(M+H)+.

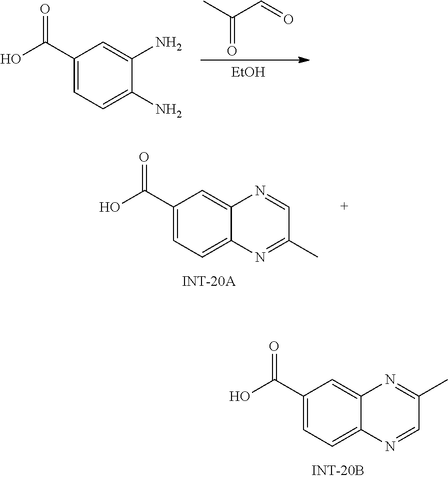

### 2-Methylquinoxaline-6-carboxylic acid INT-20A and 3-methylquinoxaline-6-carboxylic acid INT-20B

A round-bottom flask containing a mixture of 3,4-diaminobenzoic acid (8.0 g, 52.6 mmol), 2-oxopropanal (7.6 g, 105 mmol) in EtOH (200 mL) was placed in oil bath heated to 80° C. and refluxed for 12 h. LCMS showed product as a main peak. The reaction mixture was concentrated and purified by silica gel chromatography eluting with DCM/EA (with EA from 0 to 60% in 30 min) to give INT-20A and INT-20B (6.7 g, 68% yield) as yellow solids and these were used as mixtures for the next step. LCMS (ESI): m/z 189.0 (M+H)+.

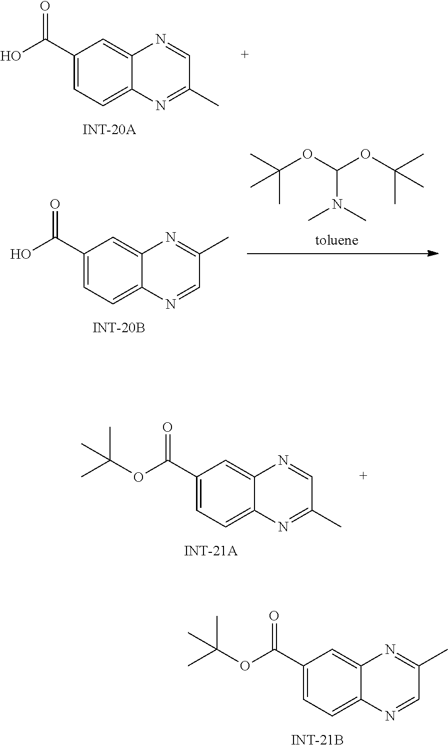

### Tert-butyl 2-methylquinoxaline-6-carboxylate INT-21A and tert-butyl 3-methylquinoxaline-6-carboxylate INT-21B

A round-bottom flask containing a mixture of INT-20A and INT-20B (6.7 g, 35.6 mmol), bis(tert-butoxy)methyl)dimethylamine (28.9 g, 142.4 mmol) in dioxane (150 ml) was placed in oil bath heated to 90° C. and stirred for 12 h. The reaction mixture was concentrated and the residue was purified by silica gel chromatography eluting with PE/EA (with EA from 0 to 20% in 30 min) to give INT-21A (2.7 g, 31% yield) and INT-21B (2.2 g, 25% yield) as yellow solid. LCMS (ESI): m/z 245.1 (M+H)⁺.

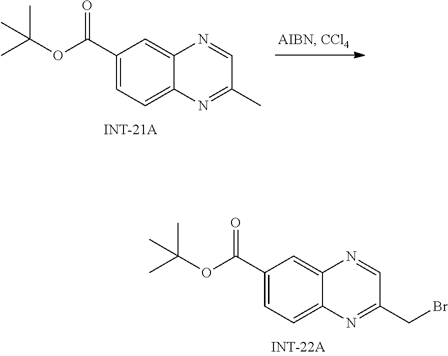 [SMILES: Cc1cnc2cc(C(=O)OC(C)(C)C)ccc2n1 ; CC(C)(C)OC(=O)c1ccc2nc(CBr)cnc2c1]

### Tert-butyl 2-(bromomethyl)quinoxaline-6-carboxylate INT-22A

Around-bottom flask containing a mixture of INT-21A (2.7 g, 11.0 mmol), NBS (2.36 g, 13.2 mmol) and AIBN (0.17 g, 1.1 mmol) in CCl₄ (50 mL) was placed in oil bath and heated to refluxed for 5 h. The reaction mixture was cooled, concentrated and purified by silica gel chromatography eluting with PE/EA (with EA from 0 to 10% in 20 min) to give INT-22A (1.5 g, 42% yield) as purple oil. LCMS (ESI): m/z 323.0 (M+H)⁺.

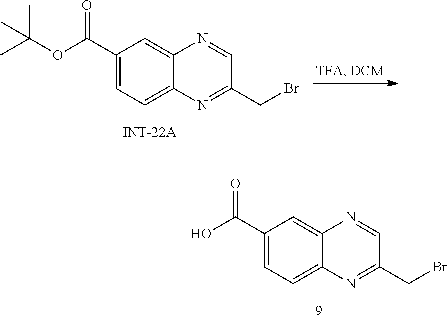

### 2-(Bromomethyl)quinoxaline-6-carboxylic acid 9

To a solution of INT-22A (1.5 g, 4.6 mmol) in DCM (30 ml) was added TFA (5.24 g,0.046 mol) at 25° C. The mixture was stirred at 25° C. for 12 h. The mixture was concentrated and freeze dried to give 9 (1.2 g, 98% yield) as purple solid. LCMS (ESI): m/z 267.0 (M+H)⁺.

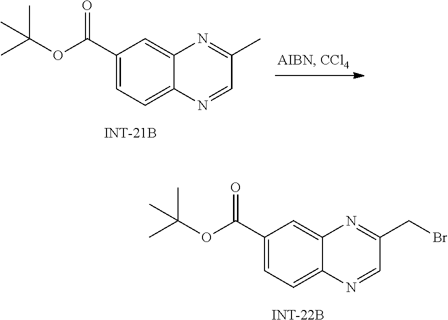 [SMILES: Cc1cnc2ccc(C(=O)OC(C)(C)C)cc2n1 ; "INT-21B" ; CC(C)(C)OC(=O)c1ccc2ncc(CBr)nc2c1 ; "INT-22B"]

### Tert-butyl 3-(bromomethyl)quinoxaline-6-carboxylate INT-22B

A round-bottom flask containing a mixture of INT-21B (2.2 g, 9.0 mmol), NBS (1.9 g, 10.8 mmol) and AIBN (147 mg, 0.9 mmol) in CCl₄ (50 mL) was placed in oil bath and heated to refluxed for 5 h. The reaction mixture was concentrated and purified by silica gel chromatography eluting with PE/EA (with EA from 0 to 10% in 20 min) to give INT-22B (1.4 g, 48% yield) as a purple oil. LCMS (ESI): m/z 323.0 (M+H)⁺.

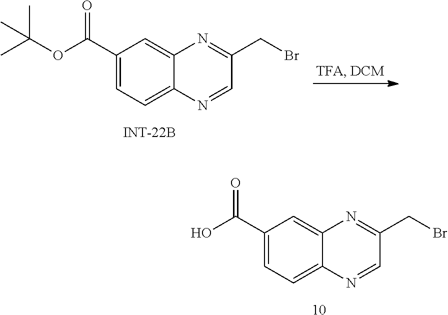

### 3-(Bromomethyl)quinoxaline-6-carboxylic acid 10

To a solution of INT-22B (1.4 g, 4.3 mmol) in DCM (30 ml) was added TFA (4.55 g, 43.0 mmol) at 25° C. The mixture was stirred at 25° C. for 12 h. The mixture was concentrated and freeze dried to give 10 (0.98 g, 85% yield) as a purple solid. LCMS (ESI): m/z 267.0 (M+H)⁺.

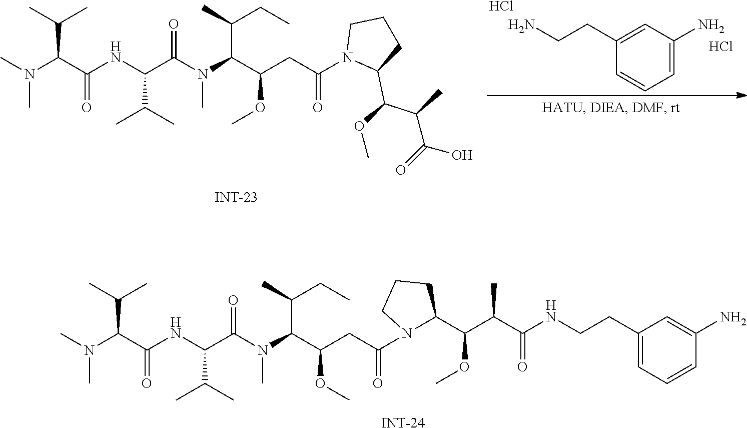

### (S)-N-((3R,4S,5S)-1-((S)-2-((1R,2R)-3-((3-aminophenethyl)amino)-1-methoxy-2-methyl-3-oxo propyl)pyrrolidin-1-yl)-3-methoxy-5-methyl-1-oxoheptan-4-yl)-2-((S)-2-(dimethylamino)-3-met hylbutanamido)-N,3-dimethylbutanamide INT-24

To a solution of 3-(2-amino-ethyl)-phenylamine dihydrochloride (1.67 g, 8.01 mmol) in DMF (100 mL) was added DIEA (4.6 mL, 26.72 mmol). The mixture was stirred at room temperature for 0.5 h, then INT-23 (4 g, 6.68 mmol) was added, followed by addition of HATU (3.3 g, 8.68 mmol). The resulting mixture was stirred at room temperature for 2 h. LCMS showed completion. The reaction was quenched by H₂O (150 mL), then extracted with EtOAc (100 mL*3). The combined organic layers were washed with H₂O (50 mL) and brine (50 mL), dried over Na₂SO₄, filtrated and concentrated to dry. The residue was purified by reverse phase column (H₂O/CH₃CN) to afford INT-24 (1 g, 99% purity) as white solid. LCMS (ESI): m/z 717.2 [M+H]⁺; HPLC: 99.48% @210 nm, Rₜ=10.72 min; ¹H NMR (400 MHz, DMSO-d₆) δ 8.09-7.96 (m, 1H), 7.82 (t, J=5.6 Hz, 1H), 6.89 (t, J=8.0 Hz, 1H), 6.42-6.36 (m, 2H), 6.33 (t, J=8.2 Hz, 1H), 4.90 (d, J=14.7 Hz, 2H), 4.79-4.61 (m, 1H), 4.61-4.48 (m, 1H), 4.04-3.94 (m, 1H), 3.88-3.80 (m, 1H), 3.77-3.70 (m, 1H), 3.61-3.48 (m, 1H), 3.46-3.36 (m, 1H), 3.29 (d, 3H), 3.27-3.22 (m, 1H), 3.18 (d, 3H), [3.15 (s, 1.5H); 3.00 (s, 1.5H)], 3.14-3.09 (m, 1H), 2.68-2.53 (m, 3H), 2.46-2.40 (m, 1H), 2.34-2.22 (m, 1H), 2.22-2.13 (m, 7H), 1.97-1.82 (m, 4H), 1.73-1.56 (m, 2H), 1.36-1.25 (m, 1H), 1.10-1.03 (m, 3H), 0.94-0.82 (m, 13H), 0.78-0.67 (m, 6H).

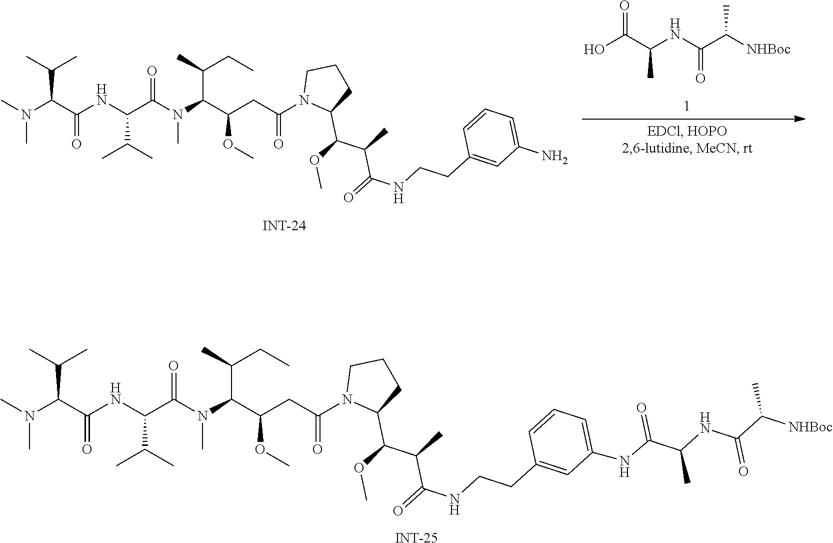

### Tert-butyl ((S)-1-(((S)-1-((3-(2-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanamido)ethyl)phenyl)amino)-1-oxopropan-2-yl)amino)-1-oxopropa n-2-yl)carbamate INT-25

To a solution of INT-24 (200 mg, 279 umol) and (S)-2-((S)-2-((tert-butoxycarbonyl)amino)propanamido)propanoic acid (84 mg, 321 umol) in 10 mL CH₃CN was added a mixture of EDCI (80 mg, 418 umol) and HOPO (46 mg, 418 umol), following by the addition of 2,6-lutidine (90 mg, 837 umol). The reaction was stirred at room temperature under a N₂ atmosphere for 16 h, LCMS showed completion. The mixture was concentrated, and the residue was purified by silica column (DCM: MeOH=100:1˜30:1, v/v) to afford INT-25 (200 mg, 75% yield) as a yellow solid. LCMS (ESI): m z 959.3 [M+H]⁺.

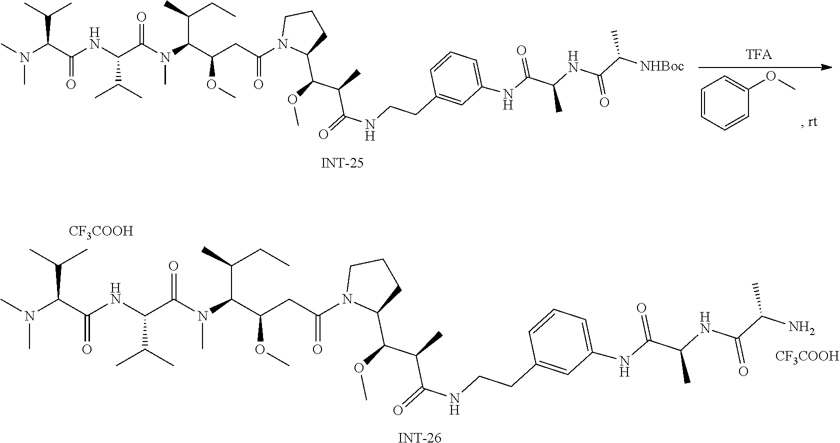

### (S)-N-((3R,4S,5S)-1-((S)-2-((1R,2R)-3-((3-((S)-2-((S)-2-aminopropanamido)propanamido)phe methyl)amino)-1-methoxy-2-methyl-3-oxopropyl)pyrrolidin-1-yl)-3-methoxy-5-methyl-1-oxohe ptan-4-yl)-2-((S)-2-(dimethylamino)-3-methylbutanamido)-N,3-dimethylbutanamide bis(2,2,2-t rifluoroacetate) INT-26

To a mixture of INT-25 (225 mg, 235 umol) and anisole (126 mg, 1.17 mmol) was added TFA (2.3 mL). The reaction was then stirred at room temperature for 10 min. TLC showed completion (DCM/MeOH=13:1, v/v; R_{f}=˜0.55 for INT-25). The mixture was diluted with 200 mL MTBE, during which time, much white solid precipated. The resulting mixture was filtrated, and the filter cake was collected and dried under reduced pressure to afford INT-26 (155 mg, 61% yield) as an off-white solid. LCMS (ESI): m z 858.8 [M+H]⁺; HPLC: 99.9% @210 nm, Rₜ=7.94 min; ¹H NMR (400 MHz, DMSO-d₆)¹H NMR (400 MHz, DMSO) δ 10.13-10.02 (m, 1H), 9.72 (s, 1H), 8.91 (s, 1H), 8.68 (d, J=7.2 Hz, 1H), 8.11 (s, 2H), 8.08-8.05 (m, 1H), 7.89 (t, J=5.4 Hz, 1H), 7.48 (s, 1H), 7.40 (t, J=7.3 Hz, 1H), 7.23-7.16 (m, 1H), 6.89 (d, J=7.6 Hz, 1H), 4.77-4.63 (m, 1H), 4.61-4.42 (m, 2H), 3.99 (s, 1H), 3.93-3.86 (m, 1H), 3.86-3.80 (m, 1H), 3.75-3.68 (m, 1H), 3.61-3.53 (m, 1H), 3.52-3.48 (m, 1H), 3.38-3.35 (m, 1H), 3.35-3.31 (m, 1H), 3.30-3.26 (m, 3H), 3.25-3.21 (m, 1H), 3.18 (d, J=4.4 Hz, 3H), [3.14 (s, 1.5H), 3.00 (s, 1.5H)], 3.13-3.08 (m, 1H), 2.84-2.70 (m, 6H), 2.70-2.60 (m, 2H), 2.46-2.42 (m, 1H), 2.32-2.25 (m, 1H), 2.22-2.15 (m, 1H), 2.04-1.97 (m, 1H), 1.92-1.82 (m, 2H), 1.82-1.73 (m, 1H), 1.72-1.65 (m, 1H), 1.64-1.50 (m, 1H), 1.39-1.31 (m, 6H), 1.30-1.19 (m, 2H), 1.06 (t, J=6.9 Hz, 3H), 0.98-0.83 (m, 15H), 0.77 (q, J=7.1 Hz, 3H)

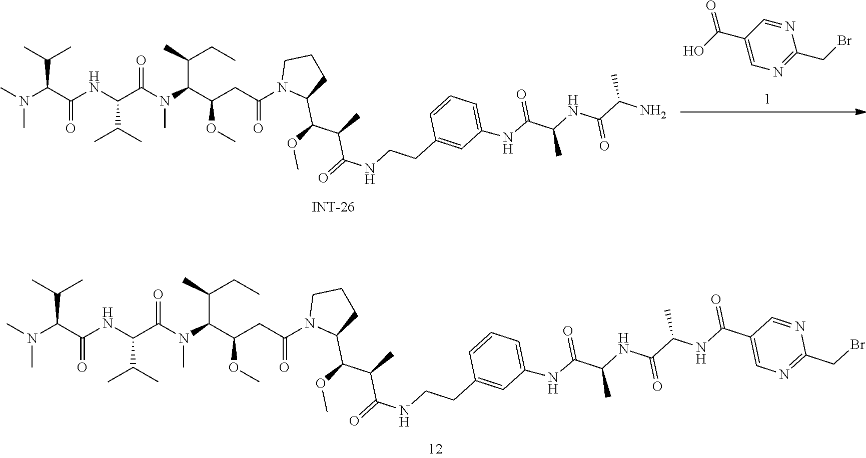

### 2-(Bromomethyl)-N-((S)-1-(((S)-1-((3-(2-((2R,3R)-3-((S)-1-((3R,4S,5S)-4-((S)-2-((S)-2-(dimeth ylamino)-3-methylbutanamido)-N,3-dimethylbutanamido)-3-methoxy-5-methylheptanoyl)pyrr olidin-2-yl)-3-methoxy-2-methylpropanamido)ethyl)phenyl)amino)-1-oxopropan-2-yl)amino)-1-oxopropan-2-yl)pyrimidine-5-carboxamide 12

To a solution of 1 (20 mg, 0.09 mmol) in DCM (20 mL) stirred under nitrogen at 25° C. was added a solution of EDC (3.6 mg, 0.02 mmol) and HOBt (3.1 mg, 0.02 mmol) in DCM (5 mL), the mixture was stirred for 5 min. Then was added a solution of INT-26 (20 mg, 0.02 mmol) in DCM (2 mL). The reaction mixture was stirred at 25° C. for 1 h. Quenched with 1% TFA (7 mL). Removed off DCM under vacuum. ACN was added until the mixture all dissolve, purified by prep-HPLC (ACN-H2O(0.11% TFA), 30%-50%) to give compound 12 (12 mg, 51%) as a white solid. LCMS (ESI): m/z 1057.4 [M+H]⁺. ¹H NMR (400 MHz,) δ 9.21-9.16 (m, 1H), 9.16-9.14 (m, 1H), 8.56 (dd, J=7.4, 6.8 Hz, 1H), 8.07-7.76 (m, 1H), 7.59-7.41 (m, 2H), 7.41-7.04 (m, 3H), 6.94-6.83 (m, 1H), 6.82-6.50 (m, 1H), 4.85-4.60 (m, 4H), 4.53-4.32 (m, 2H), 4.09-3.95 (m, 1H), 3.89-3.69 (m, 2H), 3.68-3.39 (m, 3H), 3.36 (s, 1H), 3.31 (s, 2H), 3.28 (s, 2H), 3.24-3.18 (m, 3H), 3.08 (d, J=22.7, 2H), 3.02-2.91 (m, 2H), 2.84-2.76 (m, 6H), 2.76-2.68 (m, 3H), 2.52-2.49 (m, 2H), 2.15-2.06 (m, 3H), 1.52-1.42 (m, 4H), 1.39-1.31 (m, 4H), 1.15-1.05 (m, 3H), 1.04-0.73 (m, 21H).

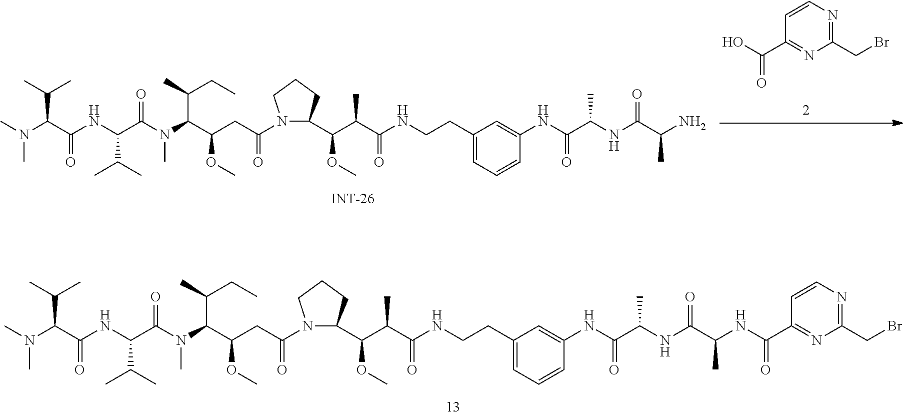 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; O=C(O)c1ccnc(CBr)n1 ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2ccnc(CBr)n2)c1)OC)N(C)C(=O)[C@@H](NC(=O)[C@H](C(C)C)N(C)C)C(C)C]

### (S)-N-((3R,4S,5S)-1-((S)-2-((1R,2R)-3-((3-((S)-2-((S)-2-aminopropanamido)propanamido)phe methyl)amino)-1-methoxy-2-methyl-3-oxopropyl)pyrrolidin-1-yl)-3-methoxy-5-methyl-1-oxohe ptan-4-yl)-2-((S)-2-(dimethylamino)-3-methylbutanamido)-N,3-dimethylbutanamide 13

To a solution of 2 (20 mg, 0.09 mmol) in DCM (20 mL) stirred under nitrogen at 25° C. was added a solution of EDC (3.6 mg, 0.02 mmol) and HOBt (3.1 mg, 0.02 mmol) in DCM (5 mL), the mixture was stirred for 5 min. Then was added a solution of INT-26 in DCM (2 mL). The reaction mixture was stirred at 25° C. for 30 min. Quenched with 1% TFA (7 mL). Removed off DCM under vacuum. ACN was added until the mixture all dissolved, purified by prep-HPLC (ACN-H₂O(0.1% TFA), 30%-50%) to give compound 13 (16 mg, 65%) as a white solid. LCMS (ESI): m/z 1057.1 [M+H]⁺. ¹H NMR (400 MHz, CD3CN) δ 9.07-8.99 (m, 1H), 8.84-8.55 (m, 2H), 8.12-7.86 (m, 1H), 7.79-7.40 (m, 3H), 7.38-7.11 (m, 2H), 6.96 (d, J=7.6 Hz, 1H), 6.75-6.68 (m, 1H), 4.80-4.63 (m, 4H), 4.62-4.44 (m, 2H), 4.12-3.93 (m, 1H), 3.86-3.67 (m, 3H), 3.66 (m, 2H), 3.47-3.42 (m, 3H), 3.40-3.25 (m, 5H), 3.25-3.22 (m, 3H), 3.14 (s, 1H), 3.07-.2.95 (m, 3H), 2.91-2.82 (m, 6H), 2.80-2.75 (m, 3H), 2.17-2.06 (m, 2H), 1.82-1.62 (m, 3H), 1.50-1.47 (m, 4H), 1.42-1.33 (m, 4H), 1.17-1.07 (m, 3H), 1.06-0.91 (m, 12H), 0.90-0.74 (m, 6H).

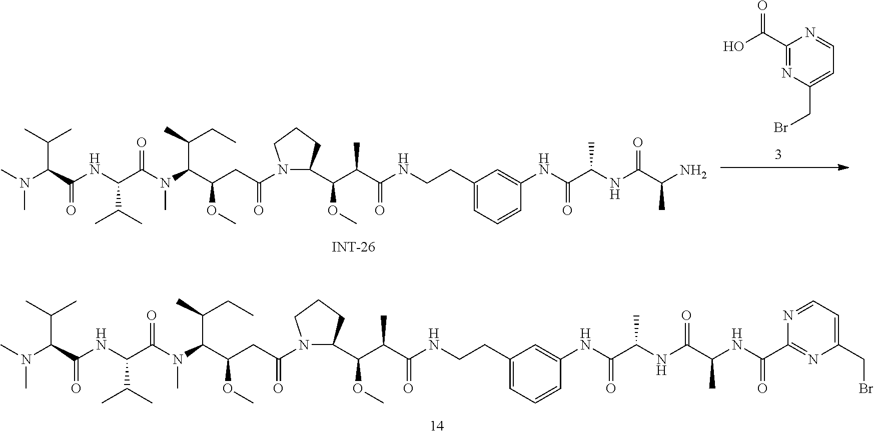 [SMILES: O=C(O)c1nccc(CBr)n1 ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2nccc(CBr)n2)c1)OC)N(C)C(=O)[C@@H](NC(=O)[C@H](C(C)C)N(C)C)C(C)C]

### 4-(Bromomethyl)-N-((S)-1-(((S)-1-((3-(2-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanamido)ethyl)phenyl)amino)-1-oxopropan-2-yl)amino)-1-oxopropan-2-yl)pyrimidine-2-carboxamide 14

To a solution of 3 (27 mg, 0.12 mmol), in DCM (20 mL) stirred under nitrogen at 25° C. was added a solution of 1-(3-Dimethylaminopropyl)-3-ethylcarbodiimide (3.6 mg, 0.02 mmol) and 1-Hydroxybenzotrizole (3.1 mg, 0.02 mmol) in DCM (5 mL), the mixture was stirred for 5 mins. Then was added a solution of INT-26 (20 mg, 0.02 mmol) in DCM (2 mL). The reaction mixture was stirred at 25° C. for 30 mins. 1% TFA (7 mL) in water was added. Removed DCM under vacuum. ACN was added until the mixture all dissolve, purified by prep-HPLC (ACN-H20(0.1% TFA), 30%-50%) to give compound 14 (11.4 mg, 46.8%) as a white solid. LCMS (ESI): m/z 1057.4 (M+H)⁺. ¹H NMR (400 MHz, CD3CN) δ 9.00-8.86 (m, 1H), 8.75-8.55 (m, 2H), 7.95-7.65 (m, 2H), 7.63-7.58 (m, 2H), 7.38-7.18 (m, 2H), 7.03-6.89 (m, 1H), 6.75-6.68 (m, 1H), 4.80-4.63 (m, 4H), 4.62-4.44 (m, 2H), 4.12-3.98 (m, 1H), 3.86-3.73 (m, 2H), 3.72-3.59 (m, 2H), 3.56-3.42 (m, 2H), 3.40-3.25 (m, 5H), 3.26-3.20 (m, 3H), 3.14 (s, 1H), 3.07-.2.95 (m, 2H), 2.91-2.82 (m, 6H), 2.83-2.70 (m, 2H), 2.55-2.43 (m, 2H), 2.17-2.06 (m, 3H), 1.82-1.62 (m, 4H), 1.50-1.47 (m, 3H), 1.42-1.33 (m, 3H), 1.33-1.24 (m, 2H), 1.13-1.05 (m, 3H), 1.06-0.91 (m, 12H), 0.88-0.74 (m, 6H).

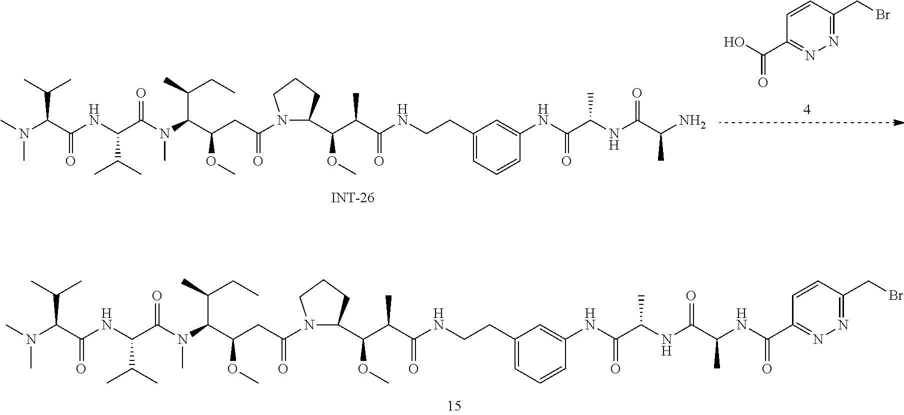 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; O=C(O)c1ccc(CBr)nn1 ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2ccc(CBr)nn2)c1)OC)N(C)C(=O)[C@@H](NC(=O)[C@H](C(C)C)N(C)C)C(C)C]

### (6-(Bromomethyl)-N-((S)-1-(((S)-1-((3-(2-((2R,3R)-3-((S)-1-((3R,4S,5S)-4-((S)-2-((S)-2-(dimet hylamino)-3-methylbutanamido)-N,3-dimethylbutanamido)-3-methoxy-5-methylheptanoyl)pyr rolidin-2-yl)-3-methoxy-2-methylpropanamido)ethyl)phenyl)amino)-1-oxopropan-2-yl)amino)-1-oxopropan-2-yl)pyridazine-3-carboxamide 15

To a solution 4 (27 mg, 0.10 mmol) in DCM (20 mL) stirred under nitrogen at 25° C. was added a solution of EDC (3.6 mg, 0.02 mmol) and HOBt (3.1 mg, 0.02 mmol) in DCM (5 mL), the mixture was stirred for 5 min. Then was added a solution INT-26 (20 mg, 0.02 mmol) in DCM (2 mL). The reaction mixture was stirred at 25° C. for 1 h. Quenched with 1% TFA (7 mL). Removed off DCM under vacuum. ACN was added until the mixture all dissolved, purified by prep-HPLC (ACN-H20(0.1% TFA), 30%-50%) to give compound 15 (9.8 mg, 39.1%) as a white solid. LCMS (ESI): m/z 1057.1 [M+H]⁺. ¹H NMR (400 MHz, CD3CN) δ 8.96-8.76 (m, 1H), 8.75-8.67 (m, 1H), 8.36-8.20 (m, 1H), 8.06-7.98 (d, J=8.7 Hz, 1H), 7.97-7.90 (m, 1H), 7.63-7.48 (m, 2H), 7.47-7.46 (m, 1H), 7.29-7.23 (m, 1H), 7.05-6.95 (m, 1H), 6.93-6.63 (m, 1H), 4.94-4.84 (m, 2H), 4.83-4.66 (m, 2H), 4.63-4.54 (m, 1H), 4.54-4.37 (m, 1H), 4.13-3.94 (m, 1H), 3.93-3.72 (m, 3H), 3.71-3.43 (m, 3H), 3.43-3.32 (m, 5H), 3.31-3.24 (m, 3H), 3.17 (s, 1H), 3.11-3. 04(m, 2H), 2.88 (s, 6H), 2.80-2.75 (m, 2H), 2.36-2.27 (m, 2H), 2.23-2.05 (d, 3H), 1.84-1.63 (m, 3H), 1.60-1.53 (m, 3H), 1.43-1.38 (m, 3H), 1.29 (s, 1H), 1.13 (d, J=6.8 Hz, 3H), 1.08-0.95 (m, 12H), 0.93-0.89 (m, 3H), 0.84-0.77 (m, 3H).

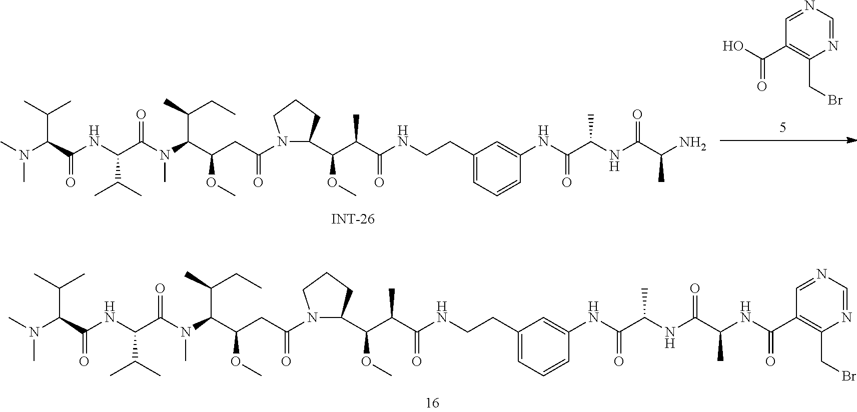 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; O=C(O)c1cncnc1CBr ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cncnc2CBr)c1)OC)N(C)C(=O)[C@@H](NC(=O)[C@H](C(C)C)N(C)C)C(C)C]

### 4-(Bromomethyl)-N-((S)-1-(((S)-1-((3-(2-((2R,3R)-3-((S)-1-((3R,4S,5S)-4-((S)-2-((S)-2-(dimeth ylamino)-3-methylbutanamido)-N,3-dimethylbutanamido)-3-methoxy-5-methylheptanoyl)pyrr olidin-2-yl)-3-methoxy-2-methylpropanamido)ethyl)phenyl)amino)-1-oxopropan-2-yl)amino)-1-oxopropan-2-yl)pyrimidine-5-carboxamide 16

To a solution of 5 (20 mg, 0.09 mmol) in DCM (20 mL) stirred under nitrogen at 25° C. was addeda solution of 1-(3-Dimethylaminopropyl)-3-ethylcarbodiimide (3.6 mg, 0.02 mmol) and 1-Hydroxybenzotrizole (3.1 mg, 0.02 mmol) in DCM (5 mL), the mixture was stirred for 5 mins. Then was added a solution of INT-26 (20 mg, 0.02 mmol) in DCM (2 mL). The reaction mixture was stirred at 25° C. for 1 h. 30% SM was remained, about 40% TM, 20% HOBt substitute byproduct on LCMS. Quenched with 10% TFA (6 mL). Removed off DCM under vacuum. ACN was added until the mixture all dissolved, purified by prep-HPLC (ACN-H₂O (0.1% TFA), 30%-50%) to give 16 (2 mg, 8.2%) as a white solid. LCMS (ESI): m/z 1057.4 [M+H]⁺. ¹H NMR (400 MHz, CD3CN) δ 9.54-9.16 (m, 2H), 9.08-8.47 (m, 2H), 8.40-8.78 (m, 2H), 7.65-7.35 (m, 3H), 7.33-7.22 (m, 1H), 7.03-6.85 (m, 1H), 4.90-4.65 (m, 4H), 4.56-4.35 (m, 2H), 4.06 (s, 3H), 3.90-3.70 (m, 1H), 3.65-3.43 (m, 3H), 3.43-3.19 (m, 10H), 3.19-3.00 (m, 4H), 2.88 (s, 7H), 2.82-2.68 (m, 4H), 1.53-1.90 (m, 4H), 1.52-1.46 (m, 3H), 1.46-1.40 (m, 2H), 1.30 (s, 1H), 1.15-1.09 (m, 3H), 1.08-0.94 (m, 12H), 0.90 (d, J=6.9 Hz, 3H), 0.86-0.79 (m, 3H).

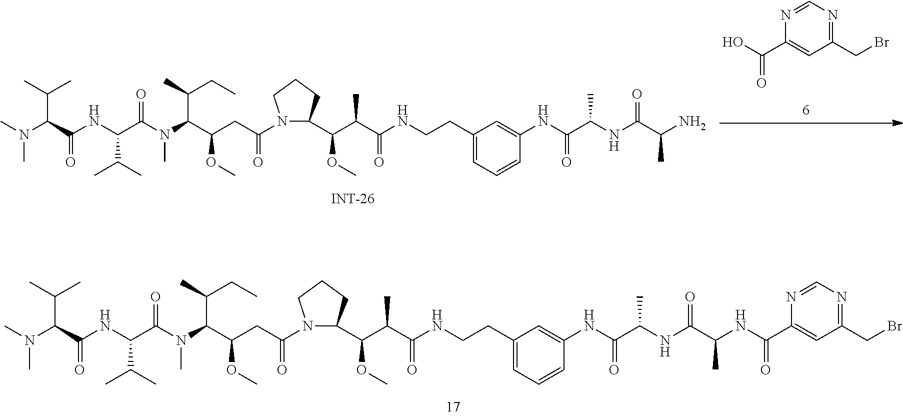 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; O=C(O)c1cc(CBr)ncn1 ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cc(CBr)ncn2)c1)OC)N(C)C(=O)[C@@H](NC(=O)[C@H](C(C)C)N(C)C)C(C)C]

### 6-(Bromomethyl)-N-((S)-1-(((S)-1-((3-(2-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanamido)ethyl)phenyl)amino)-1-oxopropan-2-yl)amino)-1-oxopropan-2-yl)pyrimidine-4-carboxamide 17

To a solution of 6 (15 mg, 0.07 mmol) in DCM (15 mL), stirred under nitrogen at 25° C. was added 1-(3-Dimethylaminopropyl)-3-ethylcarbodiimide (5 mg, 0.03 mmol) and HOBT (2.5 mg, 0.02 mmol) in DCM (3 mL), the reaction mixture was stirred for 2 mins and then was added INT-26 (20 mg, 0.02 mmol) in DCM (2 mL). The reaction mixture was stirred at 25° C. for 20 mins. Two drops of TFA was added and then 5 mL of water. Removed off DCM under vacuum. ACN was added until the mixture all dissolve, purified by prep-HPLC (ACN-H20(0.1% TFA), 30%-50%) to give 17 (12.8 mg, 47.64%) as a white solid. MS: m/z=1057.1(M⁺, ESI+). ¹H NMR (400 MHz, CD3CN) d 9.24 (s, 1H), 8.74 (d, J=26.2 Hz, 1H), 8.55 (m, 1H), 8.24-8.13 (m, 1H), 7.57 (s, 1H), 7.48 (dd, J=19.1, 12.0 Hz, 2H), 7.40 (d, J=11.9 Hz, 1H), 7.23 (dd, J=10.4, 5.1 Hz, 1H), 6.96 (d, J=7.4 Hz, 1H), 6.87-6.55 (m, 1H), 4.85-4.30 (m, 7H), 4.08-3.94 (m, 1H), 3.88-3.69 (m, 3H), 3.46 (d, J=9.6 Hz, 3H), 3.38 (s, 1H), 3.32 (q, J=5.2 Hz, 5H), 3.25 (d, J=4.7 Hz, 2H), 3.23 (s, 1H), 3.14 (s, 1H), 3.05 (d, J=12.0 Hz, 2H), 2.85 (s, 6H), 2.79-2.74 (m, 2H), 2.50 (M, 1H), 2.13-2.06 (m, 2H), 1.77 (M, 1H), 1.65 (dd, J=11.3, 6.8 Hz, 1H), 1.49 (M, 3H), 1.38 (d, J=7.1 Hz, 3H), 1.27 (s, 1H), 1.12-1.08 (m, 3H), 1.06-1.02 (m, 3H), 1.00 (d, J=6.7 Hz, 3H), 0.96-0.77 (m, 15H).

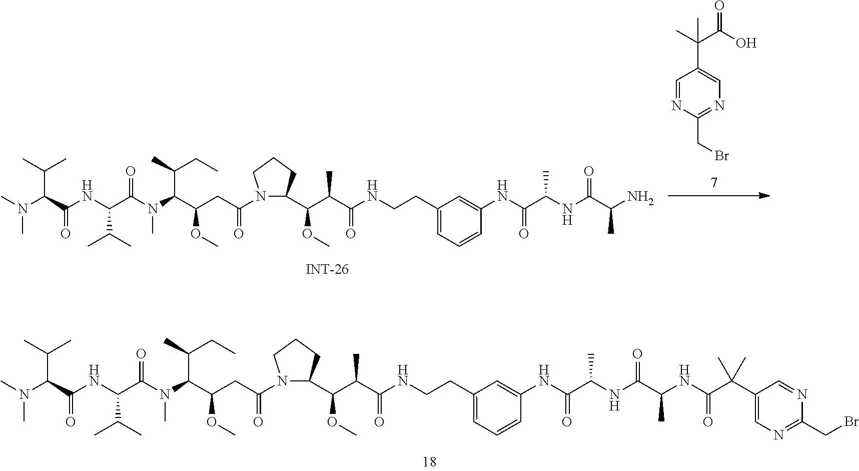 [SMILES: CC(C)(C(=O)O)c1cnc(CBr)nc1 ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)C(C)(C)c2cnc(CBr)nc2)c1)OC)N(C)C(=O)[C@@H](NC(=O)[C@H](C(C)C)N(C)C)C(C)C]

### (S)-N-((3R,4S,5S)-1-((S)-2-((1R,2R)-3-((3-((S)-2-((S)-2-(2-(2-(bromomethyl)pyrimidin-5-yl)-2-methylpropanamido)propanamido)propanamido)phenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 18

To a solution of 7 (27 mg, 0.1 mmol), in DCM (20 mL) stirred under nitrogen at 25° C. was added a solution of 1-(3-Dimethylaminopropyl)-3-ethylcarbodiimide (6.7 mg, 0.03 mmol) and 1-Hydroxybenzotrizole (2.4 mg, 0.02 mmol) in DCM (5 mL), the mixture was stirred for 5 mins. Then was added a solution of INT-26 (30 mg, 0.03 mmol) in DCM (5 mL). The reaction mixture was stirred at 25° C. for 30 mins. Two drops of TFA were added and then 5 mL of water. Removed DCM under vacuum. ACN was added until the mixture all dissolve, purified by prep-HPLC (ACN-H20(0.1% TFA), 30%-50%) to give 18 (16.5 mg, 38.97%) as a white solid. LCMS (ESI): m/z 1099.6 [M+H]⁺. 1H NMR (400 MHz, CD3CN) δ 8.81-8.75 (m, 2H), 8.59 (d, J 25.6 Hz, 1H), 7.55-7.37 (m, 3H), 7.20 (dd, J=10.3, 5.3 Hz, 1H), 7.03 (d, J=6.8 Hz, 1H), 6.93 (d, J=7.7 Hz, 1H), 6.82 (d, J=5.3 Hz, 1H), 6.68 (d, J=16.1 Hz, 1H), 4.86-4.67 (m, 2H), 4.57 (d, J=1.7 Hz, 2H), 4.40-4.20 (m, 2H), 4.04 (s, 1H), 3.79 (d, J=9.0 Hz, 1H), 3.69 (t, J 5.1 Hz, 1H), 3.47 (d, J=9.5 Hz, 1H), 3.37 (d, J=6.6 Hz, 1H), 3.34 (s, 2H), 3.31 (d, J=3.1 Hz, 2H), 3.23 (d, J=6.8 Hz, 3H), 3.15 (s, 1H), 3.02 (s, 2H), 2.84 (d, J=1.8 Hz, 6H), 2.77-2.64 (m, 3H), 2.48 (d, J=6.8 Hz, 2H), 2.37-2.29 (m, 1H), 2.18-2.03 (m, 2H), 1.92-1.88 (m, 1H), 1.80-1.74 (m, 1H), 1.67 (dd, J=13.2, 7.1 Hz, 1H), 1.61 (d, J=2.0 Hz, 6H), 1.58 (t, J=3.2 Hz, 1H), 1.34 (dd, J=16.4, 7.2 Hz, 6H), 1.27 (s, 1H), 1.17-1.08 (m, 3H), 1.02 (qd, J=12.0, 5.1 Hz, 6H), 0.95 (dt, J=6.5, 3.1 Hz, 6H), 0.91-0.71 (m, 9H).

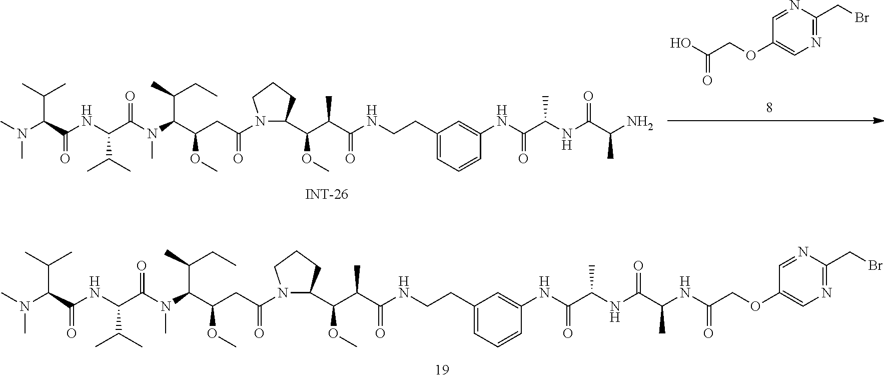 [SMILES: O=C(O)COc1cnc(CBr)nc1 ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)N)c1)OC)N(C)C(=O)[C@@H](NC(=O)[C@H](C(C)C)N(C)C)C(C)C ; "INT-26" ; CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)COc2cnc(CBr)nc2)c1)OC)N(C)C(=O)[C@@H](NC(=O)[C@H](C(C)C)N(C)C)C(C)C]

### (S)-N-((3R,4S,5S)-1-((S)-2-((JR,2R)-3-((3-((S)-2-((S)-2-(2-((2-(bromomethyl)pyrimidin-5-yl)oxy)acetamido)propanamido)propanamido)phenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 19

To a solution of 8 (27 mg, 0.10 mmol) in DCM (20 mL) stirred under nitrogen at 25° C. was added a solution of EDC (3.6 mg, 0.02 mmol) and HOBt (3.1 mg, 0.02 mmol) in DCM (5 mL), the mixture was stirred for 5 min. Then was added a solution of INT-26 (20 mg, 0.02 mmol) in DCM (2 mL). The reaction mixture was stirred at 25° C. for 1 h. Quenched with 1% TFA (7 mL). Removed off DCM under vacuum. ACN was added until the mixture all dissolved, purified by prep-HPLC (CAN-H₂O(0.1% TFA), 30%-50%) to give 19 (9.8 mg, 39.1%) as a white solid. LCMS (ESI): m/z 1087.1 [M+H]⁺. ¹H NMR (400 MHz, CD3CN) δ 8.66-8.57 (m, 1H), 8.55-8.46 (m, 2H), 8.05-7.98 (m, 1H), 7.68-7.61 (m, 1H), 7.53-7.42 (m, 2H), 7.38-7.25 (m, 1H), 7.24-7.17 (m, 1H), 6.97-6.92(m 1H), 6.78-6.66 (m, 1H), 4.85-4.64 (m, 4H), 4.59 (d, J=14.2 Hz, 2H), 4.51-4.33 (m, 2H), 4.04 (s, 1H), 3.87-3.75 (m, 2H), 3.73-363 (m, 3H), 3.42-3.29 (m, 5H), 3.27-3.23 (m, 2H), 3.17-3.14 (s, 1H), 3.10-3.02 (m, 2H), 2.87 (s, 6H), 2.78-2.71 (m, 2H), 2.56-2.48 (m, 2H), 2.18-2.06 (m, 4H), 1.83-1.75 (s, 2H), 1.74-1.48 (m, 4H), 1.48-1.37 (m, 6H), 1.19-1.10 (m, 3H), 1.07-0.77 (m, 18H).

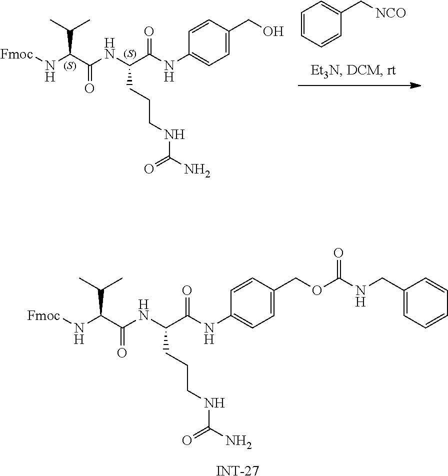

### (9H-fluoren-9-yl)methyl((S)-1-(((S)-1-((4-(((benzylcarbamoyl)oxy)methyl)phenyl)amino)-1-ox o-5-ureidopentan-2-yl)amino)-3-methyl-1-oxobutan-2-yl)carbamate INT-27

To a solution of (9H-fluoren-9-yl)methyl((S)-1-(((S)-1-((4-(hydroxymethyl)phenyl)amino)-1-oxo-5-ureidopentan-2-yl)amino)-3-methyl-1-oxobutan-2-yl)carbamate (2000 mg, 3.32 mmol) in DMF (30 mL) was added (isocyanatomethyl)benzene (883 mg, 6.64 mmol) and CuCl (448 mg, 3.32 mmol) at 25° C. the reaction was stirred at 25° C. for 24 h. The mixture was diluted with water (60 mL), filtered to give crude INT-27 (2.6 g) as a blue solid.

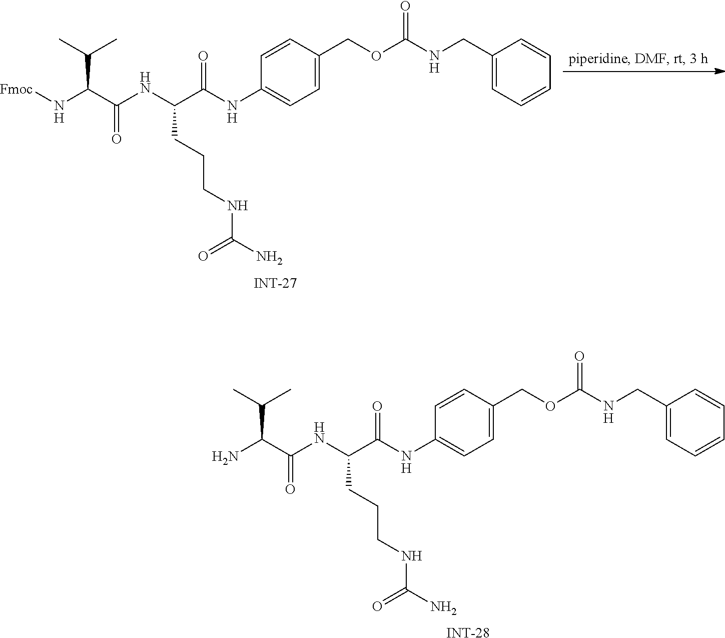

### 4-((S)-2-((S)-2-Amino-3-methylbutanamido)-5-ureidopentanamido)benzyl benzylcarbamate IN T-28

To a solution of INT-27 (2000 mg, 2.72 mmol) in DMF (20 mL) was added piperidine (1160 mg, 13.59 mmol), the reaction was stirred at RT for 2 h. The mixture was diluted with water (40 mL), the filtrate was concentrated to give desired crude INT-28 (1.5 g) as a blue solid.

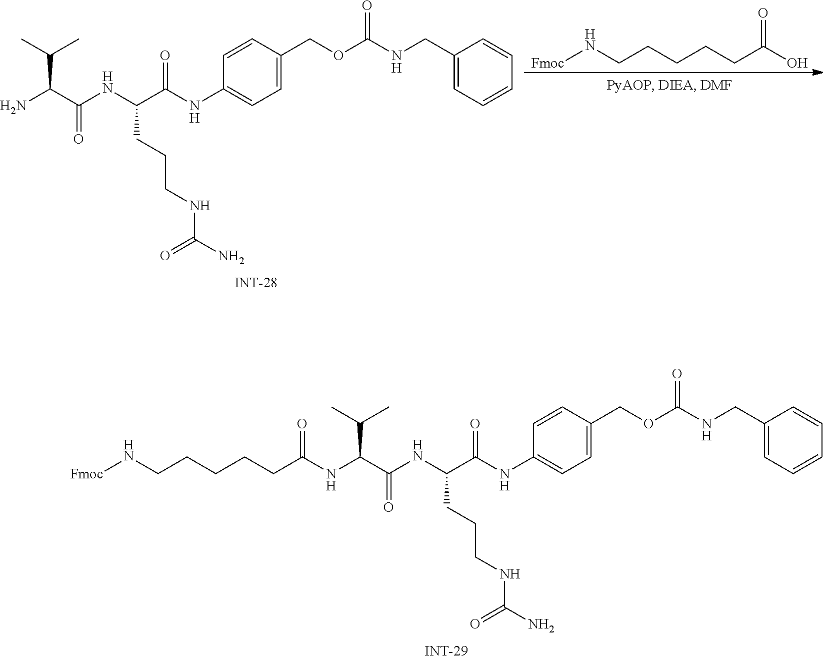

### 4-((12S,15S)-1-(9H-Fluoren-9-yl)-12-isopropyl-3,10,13-trioxo-15-(3-ureidopropyl)-2-oxa-4,11,14-triazahexadecan-16-amido)benzyl benzylcarbamate INT-29

To a solution of INT-28 (1.5 g, 2.93 mmol), 6-((((9H-fluoren-9-yl)methoxy)carbonyl)amino)hexanoic acid (1.04 g, 2.93 mmol) and PyAOP (1.68 g, 3.22 mmol) in DMF (20 mL) was added DIEA (567 mg, 4.39 mmol), the reaction was stirred 25° C. for 2 h. The mixture was diluted with water (40 mL), filtered to give the crude INT-29 (2.5 g) as a blue solid.

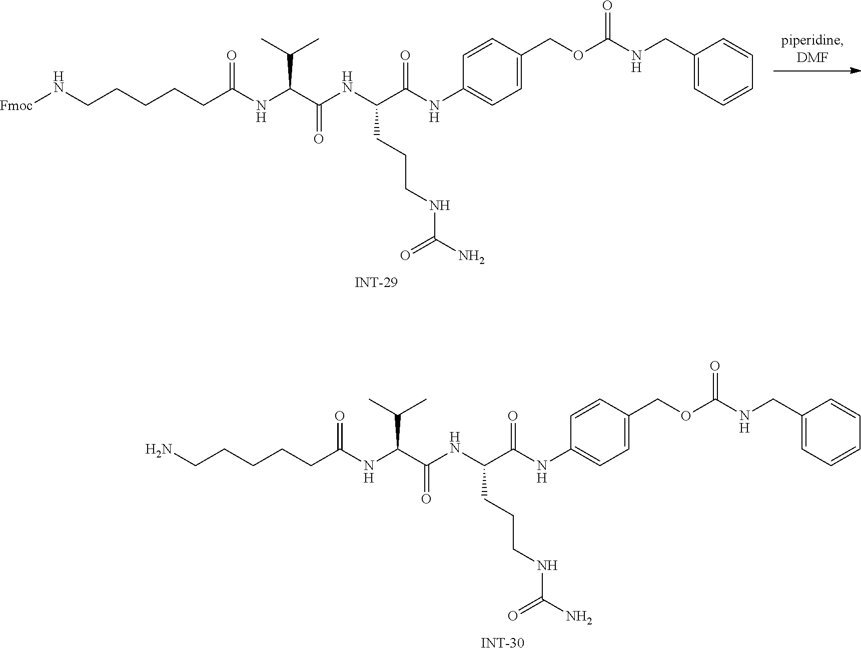

### 4-((S)-2-((S)-2-(6-Aminohexanamido)-3-methylbutanamido)-5-ureidopentanamido)benzyl ben zylcarbamate INT-30

To a solution of INT-29 (2.5 g, 2.94 mmol) in DMF (30 mL) was added peperidine (1.3 g, 14.72 mmol), the reaction was stirred at RT for 2 h. The mixture was diluted with water (60 mL), filtered, the cake was washed with EtOAc, filtered to give INT-30 (1.6 g) as a blue solid.

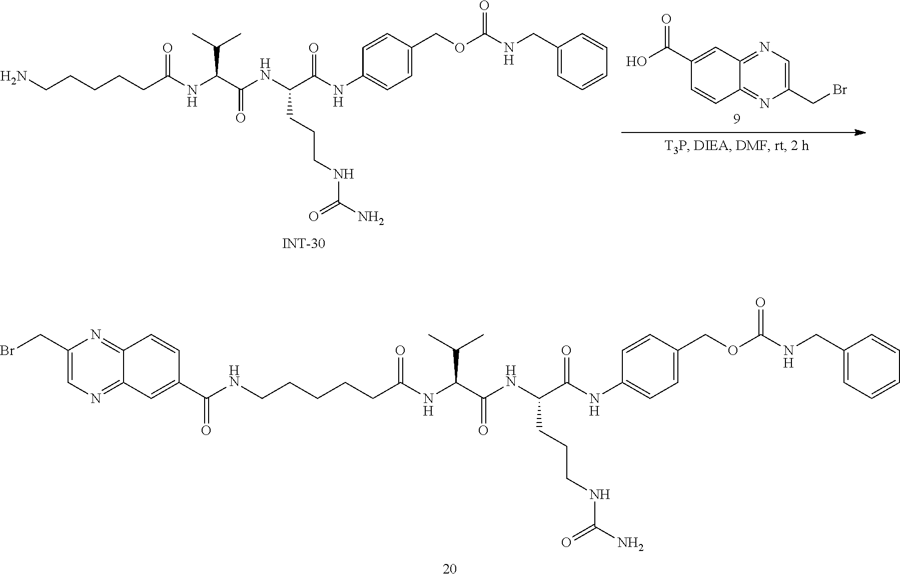

### 4-((S)-2-((S)-2-(6-(2-(bromomethyl)quinoxaline-6-carboxamido)hexanamido)-3-methylbutana mido)-5-ureidopentanamido)benzyl benzylcarbamate 20

To a solution of INT-30 (100 mg, 0.16 mmol), 9 (51 mg, 0.19 mmol) and T3P (152 mg, 0.48 mmol) in DMF (1.5 mL) stirred under nitrogen at 25° C. was added a solution of DIEA (62 mg, 0.48 mmol) in DMF (0.5 mL). The reaction mixture was stirred at 25° C. for 2 h. water (1 mL) was added, the solution was purified by prep-HPLC (ACN-H20(0.11% TFA), 30%-60%) to give 20 (5.3 mg, 3.63%) as a white solid. LCMS (ESI): m/z 897 (M+Na)+. LCMS (ESI): m/z 874 (M+H)+. 1H NMR (400 MHz, DMF) δ 10.03 (s, 1H), 9.25 (s, 1H), 8.89 (t, J=5.4 Hz, 1H), 8.71 (d, J=1.8 Hz, 1H), 8.41 (dd, J=8.8, 1.9 Hz, 1H), 8.17 (d, J=8.7 Hz, 1H), 8.12 (d, J=7.7 Hz, 1H), 7.89 (d, J=8.2 Hz, 1H), 7.74 (d, J=8.4 Hz, 2H), 7.68 (t, J=6.0 Hz, 1H), 7.38-7.31 (m, 6H), 7.29-7.22 (m, 1H), 6.21 (s, 1H), 5.66 (s, 2H), 5.07-5.02 (m, 4H), 4.61 (t, J=10.6 Hz, 1H), 4.39-4.28 (m, 3H), 3.45 (dd, J=13.9, 7.6 Hz, 2H), 3.25 (dd, J=13.5, 6.6 Hz, 1H), 3.06 (dd, J=13.6, 6.0 Hz, 1H), 2.39-2.26 (m, 2H), 2.14 (dd, J=13.3, 6.6 Hz, 1H), 1.92-1.82 (m, 1H), 1.73-1.64 (m, 5H), 1.53 (dd, J=14.0, 6.9 Hz, 2H), 1.45 (dd, J=14.6, 7.4 Hz, 2H), 0.94 (dd, J=9.6, 6.9 Hz, 6H).

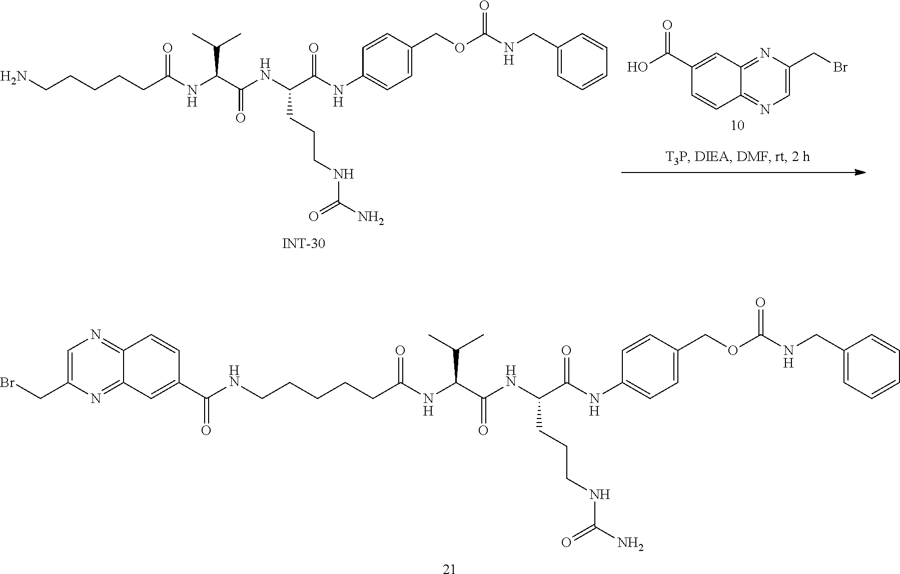

### 4-((S)-2-((S)-2-(6-(3-(Bromomethyl)quinoxaline-6-carboxamido)hexanamido)-3-methylbutana mido)-5-ureidopentanamido)benzyl benzylcarbamate 21

To a solution of INT-30 (100 mg, 0.16 mmol), 10 (51 mg, 0.19 mmol) and T₃P (152 mg, 0.48 mmol) in DMF (1.5 mL) stirred under nitrogen at 25° C. was added a solution of DIEA (62 mg, 0.48 mmol) in DMF (0.5 mL). The reaction mixture was stirred at 25° C. for 2 h. water (1 mL) was added, the solution was purified by prep-HPLC (ACN-H20(0.1% TFA), 30%-60%) to give compound 21 (2 mg, 1.31%) as a white solid. LCMS (ESI): m/z 897 (M+Na)+. LCMS (ESI): m/z 874 (M+H)+. 1H NMR (400 MHz, DMF) δ 10.03 (s, 1H), 9.24 (s, 1H), 8.88 (t, J=5.4 Hz, 1H), 8.66 (d, J=1.7 Hz, 1H), 8.39 (dd, J=8.7, 1.9 Hz, 1H), 8.26-8.18 (m, 1H), 8.11 (d, J=7.6 Hz, 1H), 7.89 (d, J=8.3 Hz, 1H), 7.74 (d, J=8.4 Hz, 2H), 7.68 (t, J=6.1 Hz, 1H), 7.35-7.30 (m, 6H), 7.25 (dd, J=8.8, 4.5 Hz, 1H), 6.21 (t, J=5.8 Hz, 1H), 5.66 (s, 2H), 5.04 (d, J=3.7 Hz, 4H), 4.66-4.55 (m, 1H), 4.38-4.29 (m, 3H), 3.47-3.44 (m, 2H), 3.25 (dd, J=13.3, 6.8 Hz, 1H), 3.06 (dd, J=13.4, 6.2 Hz, 1H), 2.38-2.29 (m, 2H), 2.15-2.12 (m, 1H), 1.93-1.84 (m, 1H), 1.73-1.65 (m, 5H), 1.57-1.51 (m, 2H), 1.49-1.41 (m, 2H), 0.94 (dd, J=9.6, 6.9 Hz, 6H).

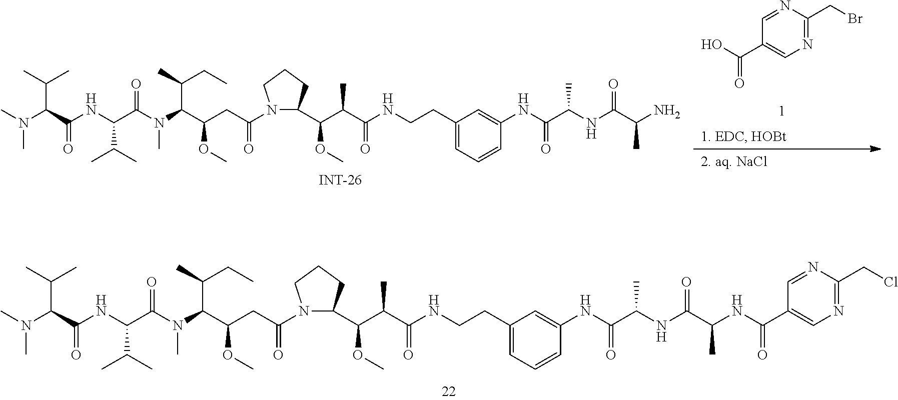

### (S)-N-((3R,4S,5S)-1-((S)-2-((1R,2R)-3-((3-((S)-2-((S)-2-Aminopropanamido)propanamido)phe methyl)amino)-1-methoxy-2-methyl-3-oxopropyl)pyrrolidin-1-yl)-3-methoxy-5-methyl-1-oxohe ptan-4-yl)-2-((S)-2-(dimethylamino)-3-methylbutanamido)-N,3-dimethylbutanamide 22

To a solution of 1 (20 mg, 0.09 mmol) in DCM (20 mL) stirred under nitrogen at 25° C. was added a solution of EDC (3.6 mg, 0.02 mmol) and HOBt (3.1 mg, 0.02 mmol) in DCM (5 mL), the mixture was stirred for 5 min. Then was added a solution of INT-26 (20 mg, 0.02 mmol) in DCM (2 mL). The reaction mixture was stirred at 25° C. for 30 min. Quenched with 1% TFA (2 mL), then was added 2 mL of saturated brine, stirred for 30 min. Removed off DCM under vacuum. ACN was added until the mixture all dissolved, purified by prep-HPLC (ACN-H20(0.1% TFA), 30%-50%) to give 22 (12 mg, 51%) as a white solid. LCMS (ESI): m/z 1013.5 [M+H]⁺. ¹H NMR (400 MHz, CD3CN) δ 9.28-9.18 (m, 2H), 9.08-8.45 (m, 1H), 8.14-7.80 (m, 1H), 7.75-7.22 (m, 5H), 7.01-6.93 (m, 1H), 6.73-6.55 (m, 1H), 4.88-4.68 (m, 4H), 4.56-4.44 (m, 2H), 4.12-4.02 (m, 1H), 3.88-3.76 (m, 2H), 3.76-3.63 (m, 2H), 3.53-3.44 (m, 2H), 3.43-3.32 (m, 6H), 3.31-3.23 (m, 4H), 3.18-3.09 (m, 2H), 3.04 (s, 2H), 2.87 (s, 7H), 2.81-2.73 (m, 4H), 1.93-1.55 (m, 4H), 1.55-1.48 (m, 3H), 1.45-1.28 (m, 4H), 1.16-0.80 (m, 21H).

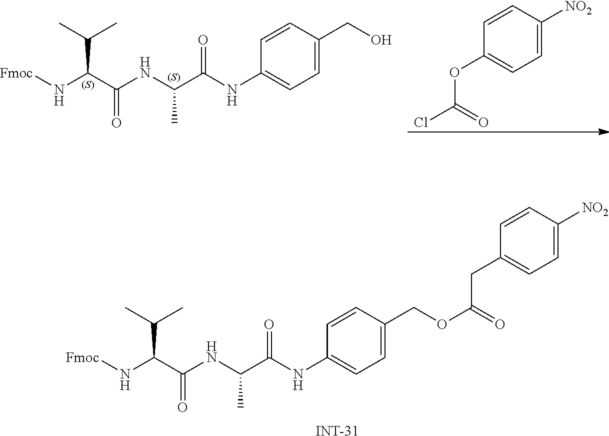

### (9H-Fluoren-9-yl)methyl((S)-3-methyl-1-(((S)-1-((4-((((4-nitrophenoxy)carbonyl)oxy)methyl)p henyl)amino)-1-oxopropan-2-yl)amino)-1-oxobutan-2-yl)carbamate INT-31

To a solution of 4-nitrophenyl chloroformate (1951 mg, 9.7 mmol) in THE (10 mL) stirred under nitrogen at 25° C. was added a solution of (9H-fluoren-9-yl)methyl((S)-1-(((S)-1-((4-(hydroxymethyl)phenyl)amino)-1-oxopropan-2-yl)amino)-3-methyl-1-oxobutan-2-yl)carbamate (2000 mg, 3.9 mmol) and trimethylamine (1225 mg, 15.5 mmol) in THE (10 mL) dropwise. The reaction mixture was stirred at 25° C. for 5 h. Diluted with MeOH (5 mL), Evaporated and the residues was purified by Flash Chromatography (petroleum ether: EtOAc=5/2), to give INT-31 (2200 mg, 82.5%) as a yellow solid. LCMS (ESI): m/z 702.8 (M+Na)⁺.

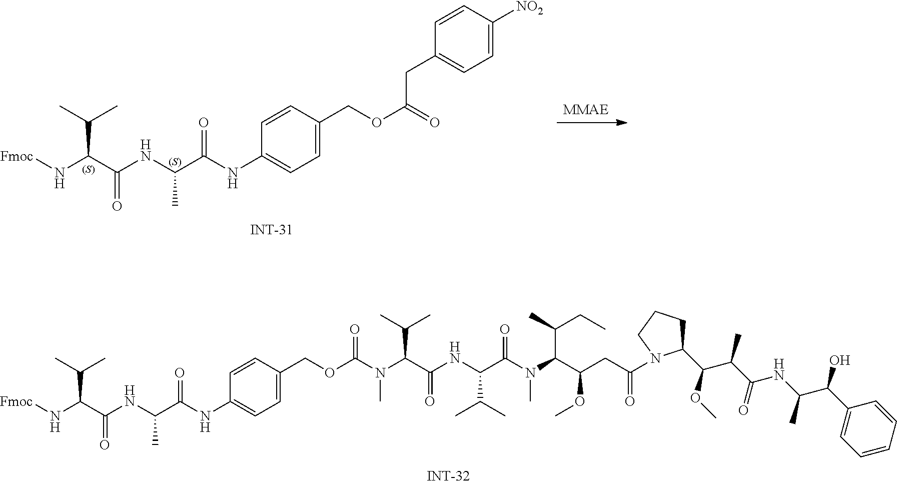

### 4-((S)-2-((S)-2-((((9H-Fluoren-9-yl)methoxy)carbonyl)amino)-3-methylbutanamido)propanam ido)benzyl((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxohept an-4-yl)(methyl)amino)-3-methyl-1-oxobutan-2-yl)amino)-3-methyl-1-oxobutan-2-yl)(methyl)c arbamate INT-32

To a solution of INT-31 (2080 mg, 3.0 mmol), (S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (MMAE; CAS 474645-27-7) (2410 mg, 3.4 mmol) and HOBT (495 mg, 3.7 mmol) in DMF (15 mL) stirred at 25° C. under nitrogen was added pyridine (3 mL). The reaction mixture was stirred at 25° C. for 12 h. Evaporated by Vacuum oil pump, the residues was purified by Flash Chromatography (DCM/MeOH=20/1), to give INT-32 (1500 mg, 38.61%) as an off-white solid. LCMS (ESI): m/z 1260 (M+H)+.

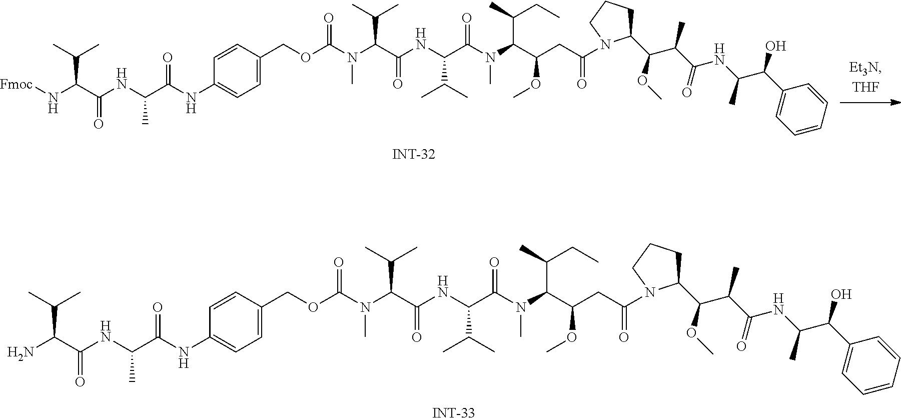

### 4-((S)-2-((S)-2-Amino-3-methylbutanamido)propanamido)benzyl((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-ox opropyl)pyrrolidin-1-yl)-3-methoxy-5-methyl-1-oxoheptan-4-yl)(methyl)amino)-3-methyl-1-ox obutan-2-yl)amino)-3-methyl-1-oxobutan-2-yl)(methyl)carbamate INT-33

To a solution of INT-32 (1500 mg, 1.2 mmol) in THE (10 mL) stirred at 25° C. was added Et₃N (2.5 mL). The reaction mixture was stirred at 60° C. for 5 h. Evaporated and the residues was washed by petroleum ether (100 mL) to give INT-33 (1050 mg, crude) as a light yellow solid. LCMS (ESI): m/z 1037.0 (M+H)+.

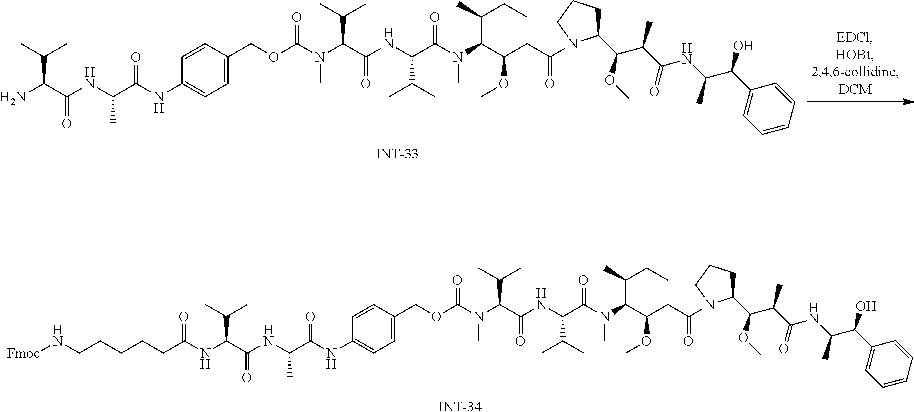

### 4-((12S,15S)-1-(9H-Fluoren-9-yl)-12-isopropyl-15-methyl-3,10,13-trioxo-2-oxa-4,11,14-triaza hexadecan-16-amido)benzyl((S)-1-(((S)-1-(((3R,4S,5S)-1-((S)-2-((1R,2R)-3-(((1S,2R)-1-hydro xy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxob utan-2-yl)(methyl)carbamate INT-34

To a solution of 6-((((9H-fluoren-9-yl)methoxy)carbonyl)amino)hexanoic acid (120 mg, 0.34 mmol), HOBt (63 mg, 0.5 mmol), EDCI (97 mg, 0.5 mmol) and 2,4,6-Collidine (123 mg, 1 mmol) in DCM (5 ml) stirred under nitrogen at 25° C. was added INT-33 (350 mg, 0.34 mmol) in DCM (2 mL). The reaction mixture was stirred at 25° C. for 2 h. Diluted with MeOH (5 mL), Evaporated and the residues was purified by Flash Chromatography (DCM/MeOH=50/3), to give INT-34 (375 mg, 80.1%) as a white solid. LCMS (ESI): m/z 1372.5 (M+H)+.

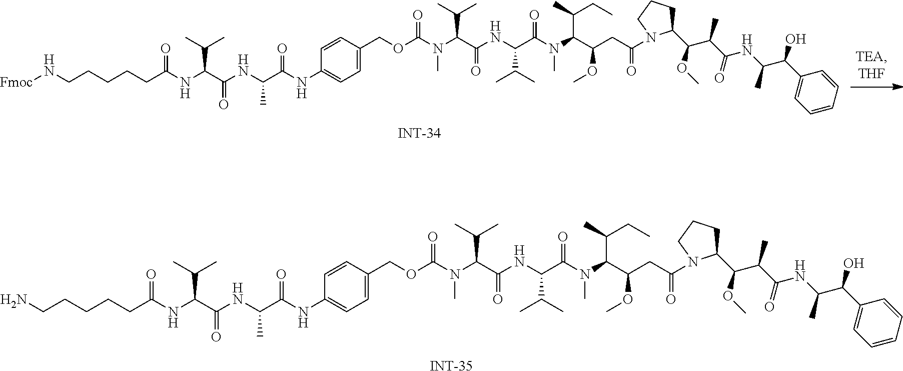

### 4-((S)-2-((S)-2-(6-Aminohexanamido)-3-methylbutanamido)propanamido)benzyl((S)-1-(((S)-1-(((3R,4S,5S)-1-((S)-2-((1R,2R)-3-(((1S,2R)-1-hydroxy-1-phenylpropan-2-yl)amino)-1-methox y-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate INT-35

To a solution of INT-34 (375 mg, 0.27 mmol) in THE (5 mL) stirred at 25° C. was added Et₃N (1.5 mL). The reaction mixture was stirred at 60° C. for 5 h. Evaporated and the residues was washed by Petroleum ether (50 mL) to give INT-35 (370 mg, crude) as a white solid. LCMS (ESI): m/z 1150.0 (M+H)+.

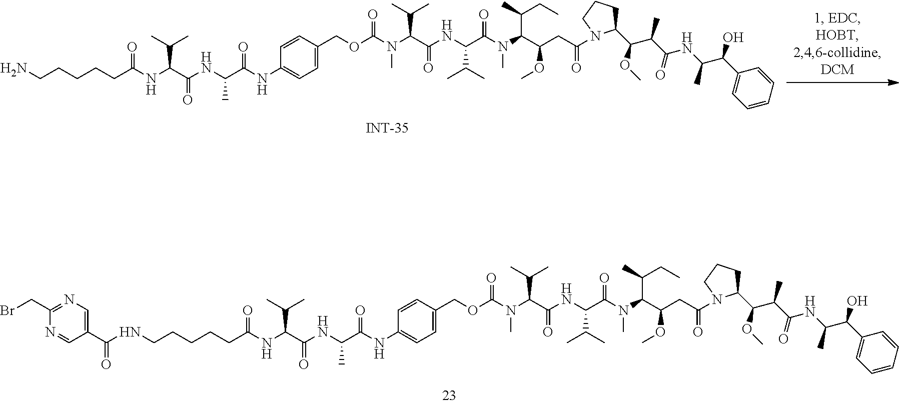 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN)C(C)C)cc1)C(C)C ; "INT-35" ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCNC(=O)c2cnc(CBr)nc2)C(C)C)cc1)C(C)C]

### 4-((S)-2-((S)-2-(6-(2-(Bromomethyl)pyrimidine-5-carboxamido)hexanamido)-3-methylbutana mido)propanamido)benzyl((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-m ethyl-1-oxoheptan-4-yl)(methyl)amino)-3-methyl-1-oxobutan-2-yl)amino)-3-methyl-1-oxobuta n-2-yl)(methyl)carbamate 23

To a solution of 1 (28 mg, 0.13 mmol), N-(3-dimethylaminopropyl)-N′-ethylcarbodiimide hydrochloride (10 mg, 0.07 mmol), HOBt (9 mg, 0.07 mmol) and 2,4,6-Collidine (32 mg, 0.26 mmol) in DCM (18 mL) stirred under nitrogen at 25° C. was added a solution of INT-35 (50 mg, 0.04 mmol) in DCM (2 mL) dropwise. The reaction mixture was stirred at 25° C. for 2 h. A solution of TFA in water (1%, 5 mL) was added, then removed off DCM under reduced pressure at room temperature, 1 mL ACN was added, the solution was applied onto prep-HPLC (ACN-H20(0.1% TFA), 30%-50%) to give 23 (7.4 mg, 11.95%) as a white solid. LCMS (ESI): m/z 675.5 (½M+H)+. LCMS (ESI): m/z 1348.8 (M+H)+. 1H NMR (400 MHz, CD3CN) δ 9.08 (s, J=7.1 Hz, 2H), 8.73 (s, 1H), 7.64 (d, J=8.5 Hz, 2H), 7.53-7.41 (m, 1H), 7.34-7.28 (m, 6H), 7.26-7.20 (m, 1H), 7.10 (d, J=7.0 Hz, 1H), 6.66 (ddd, J=38.3, 22.2, 7.6 Hz, 3H), 5.09 (dt, J=29.3, 11.3 Hz, 2H), 4.81-4.69 (m, 1H), 4.64 (s, 2H), 4.61 (s, 1H), 4.38 (dd, J=13.8, 6.7 Hz, 1H), 4.19-3.98 (m, 5H), 3.91-3.75 (m, 2H), 3.69-3.59 (m, 1H), 3.51-3.42 (m, 1H), 3.32 (s, 6H), 3.25 (s, 3H), 3.17-3.10 (m, 2H), 2.98 (s, 2H), 2.90-2.83 (m, 3H), 2.46-2.38 (m, 3H), 1.77 (dt, J=10.6, 5.3 Hz, 1H), 1.64-1.52 (m, 5H), 1.41-1.29 (m, 9H), 1.11 (dd, J=11.8, 6.8 Hz, 3H), 1.01 (dd, J=16.6, 6.9 Hz, 4H), 0.94 (dd, J=10.0, 4.6 Hz, 10H), 0.83 (dd, J=13.9, 6.6 Hz, 12H), 0.75-0.68 (m, 3H).

### Exemplary Compounds

| Compound | Structure |
|---|---|
| 1 | 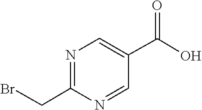 [SMILES: O=C(O)c1cnc(CBr)nc1] |
| 2 | 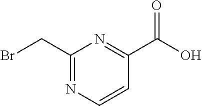 [SMILES: O=C(O)c1ccnc(CBr)n1] |
| 3 | 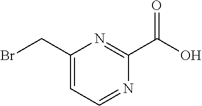 [SMILES: O=C(O)c1nccc(CBr)n1] |
| 4 | 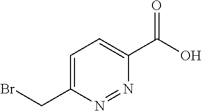 [SMILES: O=C(O)c1ccc(CBr)nn1] |
| 5 | 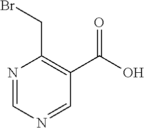 [SMILES: O=C(O)c1cncnc1CBr] |
| 6 | 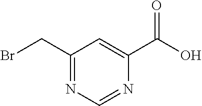 [SMILES: O=C(O)c1cc(CBr)ncn1] |
| 7 | 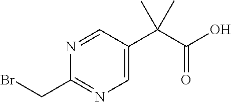 |
| 8 | 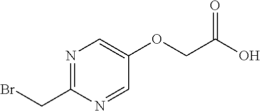 [SMILES: O=C(O)COc1cnc(CBr)nc1] |
| 9 | 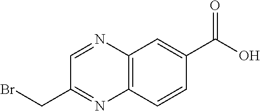 [SMILES: O=C(O)c1ccc2nc(CBr)cnc2c1] |
| 10 | 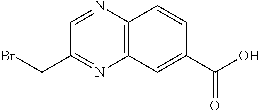 [SMILES: O=C(O)c1ccc2ncc(CBr)nc2c1] |
| 11 | 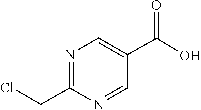 [SMILES: O=C(O)c1cnc(CCl)nc1] |
| 12 | 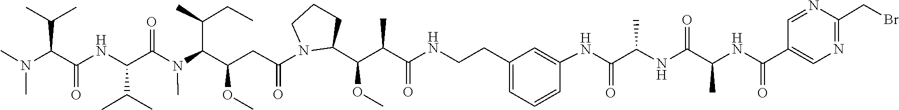 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cnc(CBr)nc2)c1)OC)N(C)C(=O)[C@@H](NC(=O)[C@H](C(C)C)N(C)C)C(C)C] |
| 13 | 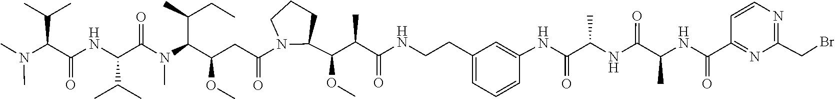 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2ccnc(CBr)n2)c1)OC)N(C)C(=O)[C@@H](NC(=O)[C@H](C(C)C)N(C)C)C(C)C] |
| 14 | 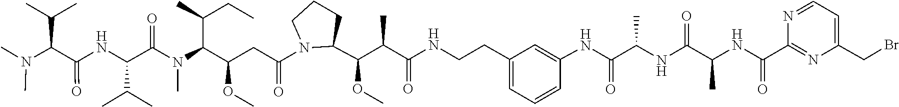 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2nccc(CBr)n2)c1)OC)N(C)C(=O)[C@@H](NC(=O)[C@H](C(C)C)N(C)C)C(C)C] |
| 15 | 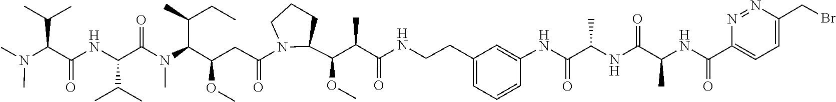 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2ccc(CBr)nn2)c1)OC)N(C)C(=O)[C@@H](NC(=O)[C@H](C(C)C)N(C)C)C(C)C] |
| 16 | 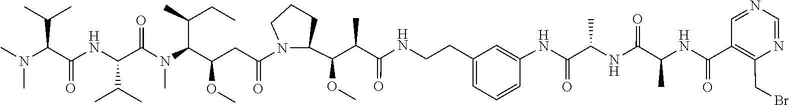 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cncnc2CBr)c1)OC)N(C)C(=O)[C@@H](NC(=O)[C@H](C(C)C)N(C)C)C(C)C] |
| 17 | 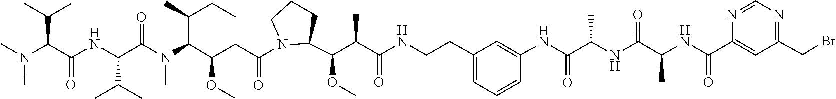 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cc(CBr)ncn2)c1)OC)N(C)C(=O)[C@@H](NC(=O)[C@H](C(C)C)N(C)C)C(C)C] |
| 18 | 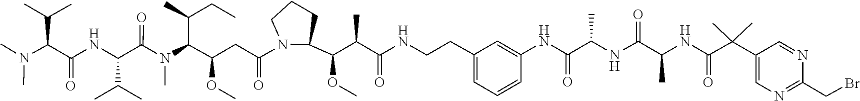 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)C(C)(C)c2cnc(CBr)nc2)c1)OC)N(C)C(=O)[C@@H](NC(=O)[C@H](C(C)C)N(C)C)C(C)C] |
| 19 | 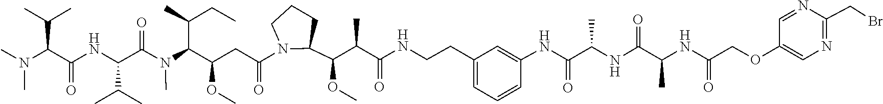 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)COc2cnc(CBr)nc2)c1)OC)N(C)C(=O)[C@@H](NC(=O)[C@H](C(C)C)N(C)C)C(C)C] |
| 20 | 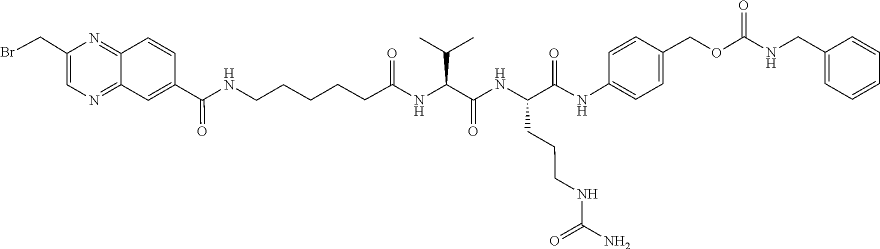 [SMILES: CC(C)[C@H](NC(=O)CCCCCNC(=O)c1ccc2nc(CBr)cnc2c1)C(=O)N[C@@H](CCCNC(N)=O)C(=O)Nc1ccc(COC(=O)NCc2ccccc2)cc1] |
| 21 | 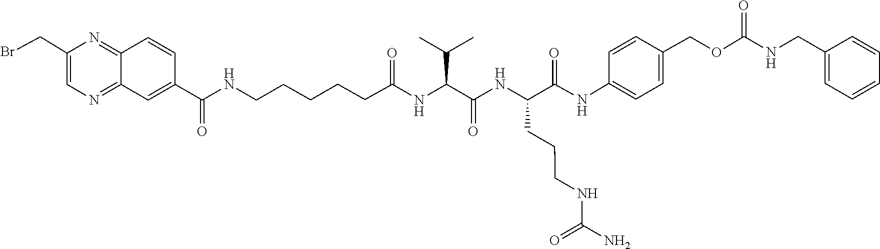 [SMILES: CC(C)[C@H](NC(=O)CCCCCNC(=O)c1ccc2nc(CBr)cnc2c1)C(=O)N[C@@H](CCCNC(N)=O)C(=O)Nc1ccc(COC(=O)NCc2ccccc2)cc1] |
| 22 | 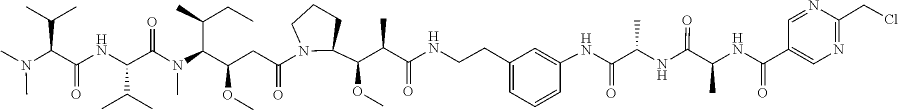 [SMILES: CC[C@H](C)[C@@H]([C@@H](CC(=O)N1CCC[C@H]1[C@H](OC)[C@@H](C)C(=O)NCCc1cccc(NC(=O)[C@H](C)NC(=O)[C@H](C)NC(=O)c2cnc(CCl)nc2)c1)OC)N(C)C(=O)[C@@H](NC(=O)[C@H](C(C)C)N(C)C)C(C)C] |
| 23 | 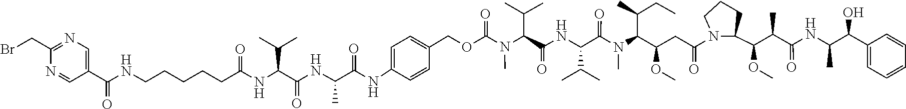 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCNC(=O)c2cnc(CBr)nc2)C(C)C)cc1)C(C)C] |

### Conjugation Methodology

Exemplary conjugation methodology is illustrated below with compound 23 to Trastuzumab (DAR4):

General conjugation protocols:
Prep Antibody into 20 mM Histidine pH 6.0 to approx. 15 mg/mL
Adjust pH to 7.2 and adjust concentration of antibody to approx. 12 mg/mL with 0.5 M Sodium phosphate buffer+50 mM EDTA to get a final phosphate buffer concentration to 100 mM Sodium Phosphate+10 mM EDTA
Reduce antibody by addition of 2.15 molar equivalence of TCEP (stock: 10 mM in water) at 37° C. for 1 hour
Bring to room temperature (20-25° C.) for 10 minute prior to drug linker addition
Prepare 10 mM stock of drug-linker in DMA
Add 5-8 molar equivalence of drug-linker
Conjugate at RT and monitor by HIC
After completion (within 3-16 h) quench by addition of 12 equivalence of N-acetyl-cysteine and left standing for 1 hour
Remove excess DL by desalting through Nap-5 column and buffer exchange into 20 mM Histidine pH 6.0 using 30 kD amicon filters.

### DAR Stability

Table 1 shows exemplary DAR stability study of Trastuzumab-MC-VC-MMAE and Trastuzumab-23 in PBS and human plasma. Greater than 40% of initial DAR is lost in human plasma with Trastuzumab-MC-VC-MMAE over 168 h. DAR loss is minimal with Trastuzumab-23 for 168 h.

TABLE 1
DAR stability (Trastuzumab-MC-VC-MMAE
and Trastuzumab-23)

| | DAR (% from T0) | | | |
|---|---|---|---|---|
| | Trastuzumab- | | | |
| | MC-VC-MMAE | | Trastuzumab-23 | |
| Time | | Human | | Human |
| (h) | PBS | plasma | PBS | plasma |
| 0 | 100 | 100 | 100 | 100 |
| 1 | 91 | 96 | 103 | 96 |
| 4 | 94 | 95 | 101 | 99 |
| 8 | 95 | 87 | 100 | 100 |
| 24 | 89 | 78 | 96 | 103 |
| 72 | 94 | 64 | 93 | 96 |
| 168 | 88 | 61 | 101 | 93 |

### Protocol for DAR Stability Assay:

Plasma IgG Depletion: endogenous IgG's were removed by Recombinant Protein A-Sepharose gel filtration. 10 mL's of Sepharose-A were used per 10 mL's plasma. Sepharose-A was washed 3× with PBS, centrifugation used to separate wash buffer from Sepharose. Plasma was mixed with Sepharose-A for 2 hours at 4° C. before plasma was removed from Sepharose by centrifugation.
Incubation: ADC's were spiked into depleted plasma from each test species and PBS to achieve 1 mg/mL concentration ADC. Samples were incubated at 37° C. Aliquots containing 20 ug's of ADC were removed at designated time points. Aliquots were frozen at −80° C. until analysis.
Capture and Elution: Protein-A beads were washed 2× with PBS and resuspended in initial volume. On a 96 well ultra low attachment plate, 15 μL of beads were added per well with 35 uL PBS and 10 μL of ADC incubated samples. Plate is mixed for 1 hour at room temp. Beads were separated and the supernatant was collected and frozen at −80° C. Beads were washed with PBS-T 3× with 250 uL PBS-T. ADC is eluted from beads with 50 μL of 100 mM Acetic acid, neutralized with 10 uL of 1.5M Tris-HCL pH 8.5. ADC's are reduced with addition 2 μL of 100 mM DTT, incubated 30 minutes at 37° C. Bead capture supernatant is diluted 1:3 with Acetonitrile and centrifuged at 17G for 10 minutes, supernatant is removed from the pelleted protein and injected on MS.
Data Analysis: DAR was determined by RP-MS, Free Payload was determined by MRM.

### Cytotoxity Date for Representative Conjugates

The tested compounds included MC-VC-PABC-MMAE Seagen drug-linker, compound 23, and MMAE.

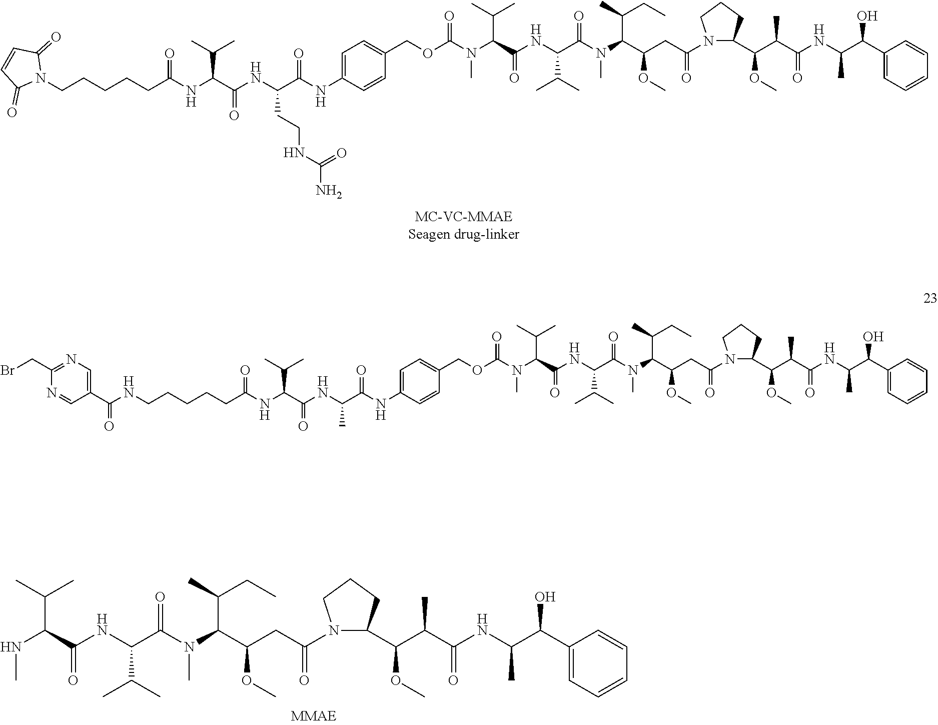

TABLE 2
In Vitro Potency of Representative Conjugates
of the Present Disclosure

| | | IC50 (nM) | |
|---|---|---|---|
| ADC | DAR | HCC1954 | SK-BR-3 |
| Trastuzumab-MC- | 3.81 | 0.04 | 0.020 |
| VC-PABC-MMAE | | | |
| Trastuzumab-23 | 3.76 | 0.06 | 0.014 |
| MMAE | — | 0.21 | 0.127 |

### Assay Protocol:

HCC1954 breast ductal carcinoma or SK-BR-3 cells (ATCC, Manassas, VA, USA) were seeded into 384-well white-walled culture plates and allowed to adhere for 2-4 hours. Cells were then treated with test articles at least in duplicate by addition of 5-fold serially diluted test articles prepared at 2× final concentration and incubated at 37° C. for 120 hours. Cell viability following treatment was determined by Cell Titer Glo 2.0 Assay (Promega, Madison, WI, USA) and normalized to non-treated controls. Dose-response relationships were analyzed using GraphPad Prism (La Jolla, CA, USA), and IC50 values were derived from non-linear regression analyses using a 4-parameter logistic equation.

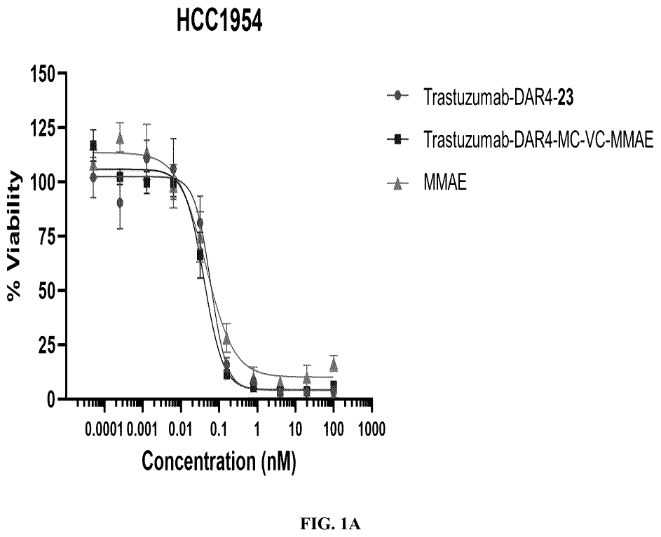

FIGS. 1A-1B showed exemplary results of in vitro assays of Trastuzumab-DAR4-23, Trastuzumab-DAR4-MC-VC-MMAE and MMAE in HCC1954 and SK-BR-3 cell lines.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, manuscripts, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A drug conjugate comprising a targeting moiety, a linker moiety, and a drug moiety, wherein the drug moiety is conjugated to the linker which is conjugated to the targeting moiety, and wherein the linker moiety has the structural formula (I^{A}) or (II^{A}): 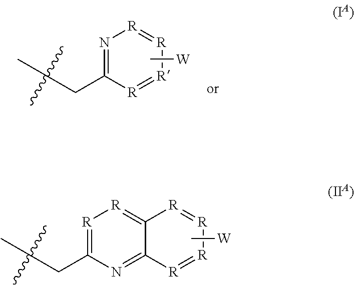
wherein:
each R is independently selected from N, CH, or C;
R′ is CH or C; and
W is selected from: 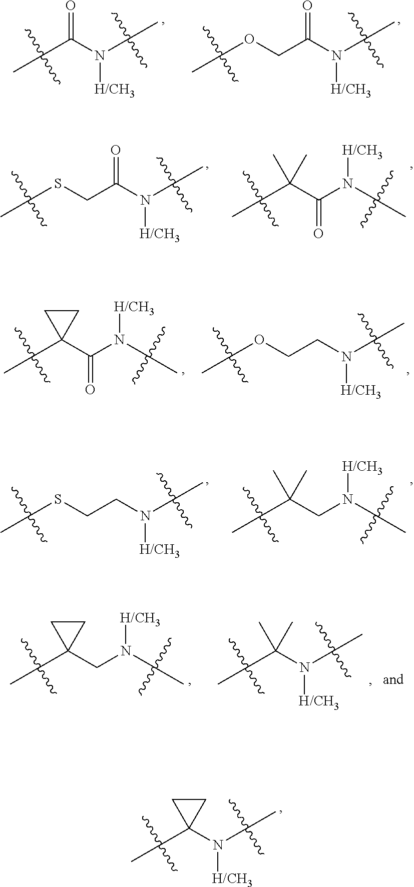
or a pharmaceutically acceptable salt thereof.

2. The drug conjugate of claim 1, wherein the linker has the structural formula (I^{B}) or (II^{B}):

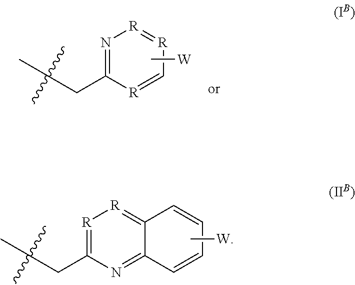

3. The drug conjugate of claim 1, wherein the linker has the structural formula (I^{C})

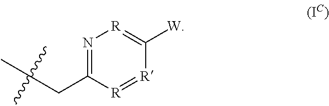

4. The drug conjugate of claim 1, wherein the linker comprises a spacer moiety and has the structural formula (III^{A}) or (IV^{A}): 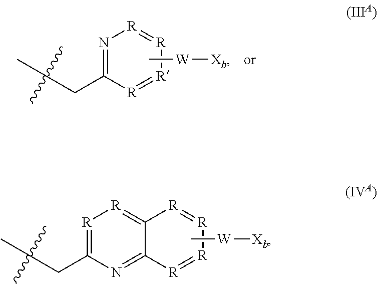
wherein X_{b} is the spacer moiety.

5. The drug conjugate of claim 4, wherein the spacer moiety is selected from the group consisting of an alkyl, a heteroalkyl, polyethylene glycol (PEG), and a peptide.

6. The drug conjugate of claim 1, wherein the linker comprises a spacer moiety and a polypeptide moiety and has the structural formula (V^{A}) or (VI^{A}): 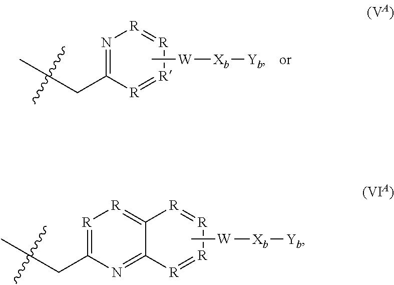
wherein Y_{b} is the polypeptide moiety.

7. The drug conjugate of claim 6, wherein the polypeptide moiety comprises 1 to 6 amino acids.

8. The drug conjugate of claim 7, wherein the amino acids are natural and/or unnatural amino acids.

9. The drug conjugate of claim 1, wherein the linker comprises a spacer moiety, a polypeptide moiety, and a self-immolative moiety and has the structural formula (VII^{A}) or (VIII^{A}): 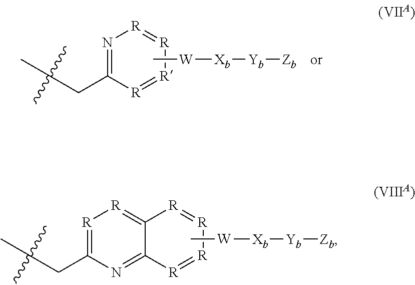
wherein Z_{b} is the self-immolative moiety.

10. The drug conjugate of claim 9, wherein the self-immolative moiety is selected from the group consisting of:

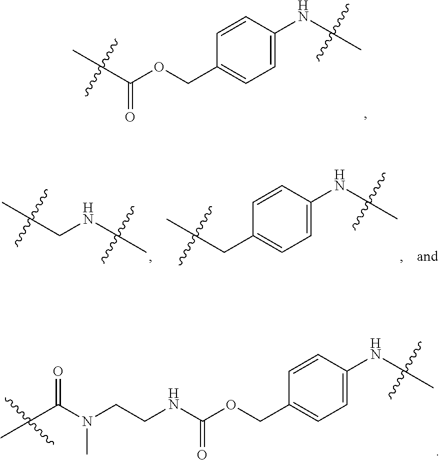

11. The drug conjugate of claim 1, wherein the linker is selected from the group consisting of:

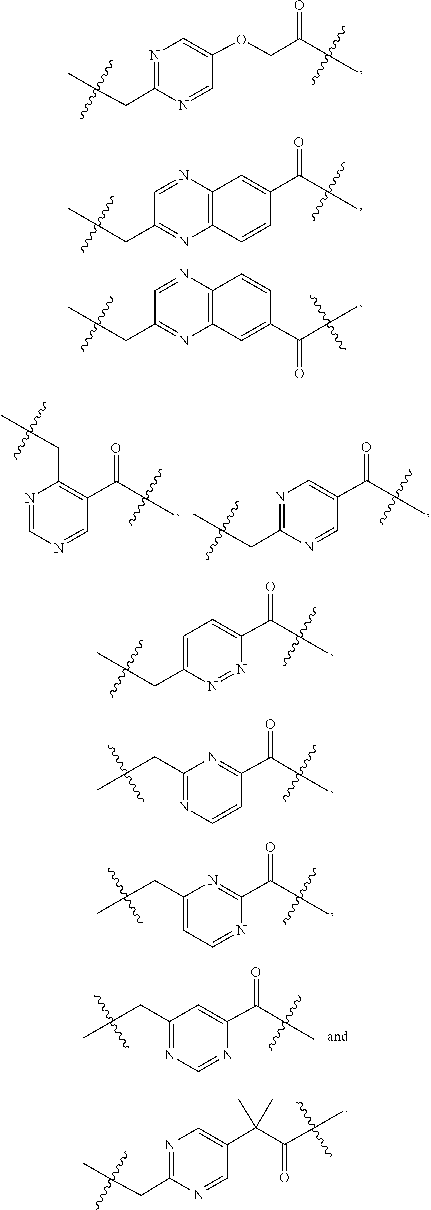

12. The drug conjugate of claim 1, wherein the drug moiety is a chemical agent selected from the group consisting of an antibiotic, an anti-cancer agent, a steroid, a TLR7/TLR9 antagonist, a polypeptide, a protein, and a nucleic acid.

13. The drug conjugate of claim 1, wherein the targeting moiety is selected from the group consisting of an antibody, small molecule, a peptide, a polypeptide, and a nucleic acid.

14. The drug conjugate of claim 1, wherein the drug conjugate has a targeting moiety to drug moiety ratio of about 1:1 to about 1:16.

15. The drug conjugate of claim 1, wherein W is:

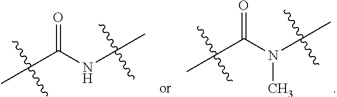

16. A composition comprising a drug conjugate of claim 1.

17. A pharmaceutical composition comprising a drug conjugate of claim 1, or a pharmaceutically acceptable salt thereof, and a pharmaceutically acceptable excipient, carrier or diluent.

18. A compound useful for forming a linker-drug conjugate, a targeting-linker conjugate, or a targeting moiety-linker-drug conjugate, the compound having a structure comprising formula (I) or (II): 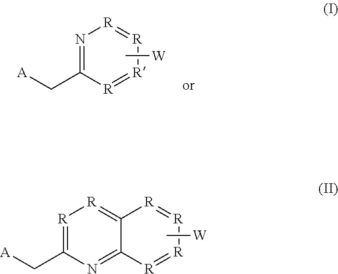
wherein:
A is Br or Cl;
each R is independently selected from N, CH, or C;
R′ is CH or C; and
W is selected from:

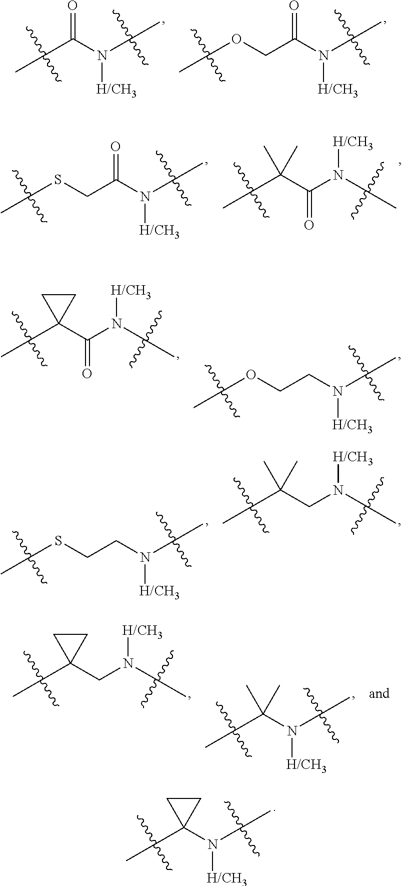

19-40. (canceled)

41. A compound useful for forming a conjugate of targeting moiety-linker-drug, the compound having a structure comprising formula (I^{a}) or (I^{a}): 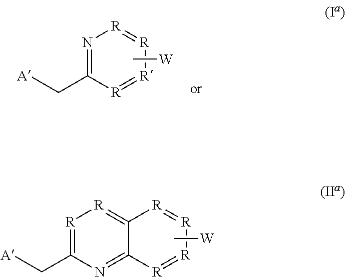
wherein:
A′ is conjugated to or comprises a targeting moiety;
each R is independently selected from N, CH, or C;
R′ is CH or C; and

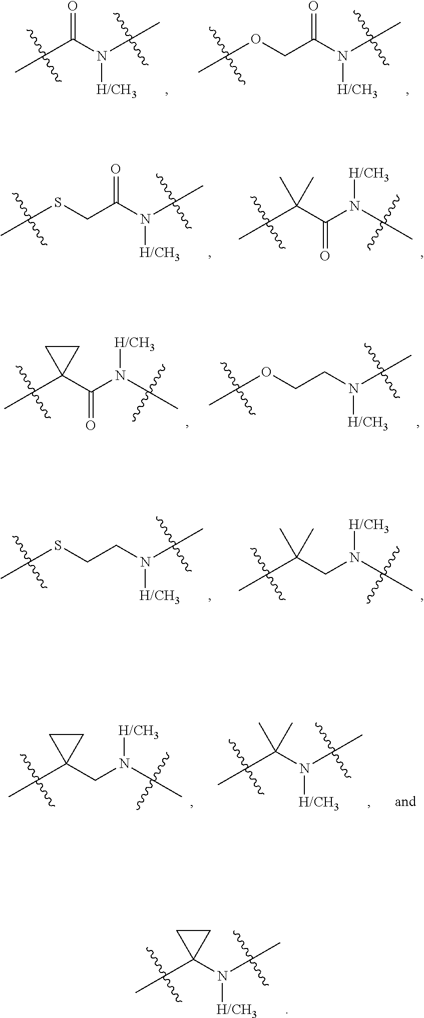

42-73. (canceled)

74. The compound of claim 1, selected from the group consisting of: [chemical structure]
